(19) United States
(12) Patent Application Publication
(10) Pub. No.: US 2022/0273809 A1
(43) Pub. Date: Sep. 1, 2022

(54) AXL ANTIBODY-DRUG CONJUGATES FOR USE IN TREATING CANCER

(71) Applicant: GENMAB A/S, Copenhagen V (DK)

(72) Inventors: Maarten JANMAAT, Utrecht (NL); Nora PENCHEVA, Utrecht (NL); Esther Cornelia Wilhelmina BREIJ, Utrecht (NL); Julia BOSHUIZEN, Amsterdam (NL); Daniel Simon PEEPER, Amstelveen (NL); Ulf FORSSMANN, Copenhagen V (DK); Tahamtan AHMADI, Princeton, NJ (US); Patricia Garrido CASTRO, Utrecht (NL)

(21) Appl. No.: 17/628,382

(22) PCT Filed: Jul. 17, 2020
(86) PCT No.: PCT/EP2020/070348

§ 371 Date: Jan. 19, 2022

Related U.S. Application Data
(60) Provisional application No. 62/962,632, filed on Jan. 17, 2020.
(60) Provisional application No. 62/931,578, filed on Nov. 6, 2019.
(60) Provisional application No. 62/876,261, filed on Jul. 19, 2019.

(51) Int. Cl. A61K 47/68; A61P 35/00; A61K 47/02

(52) U.S. Cl. CPC A61K 47/6803; A61K 47/6843; A61P 35/00; A61K 47/02; A61K 2121/00

(57) ABSTRACT

The present invention relates to conjugates of antibodies and cytotoxic agents for use in treatment of cancer in combination with an inhibitor of PD-1 or PD-L1. The antibodies are directed against Axl.

### FIELD OF INVENTION

The present invention relates to conjugates of a cytotoxic agent and an antibody capable of binding to human Axl for use in treating cancer in combination with an inhibitor of programmed cell death-1 (PD-1) and/or programmed death-ligand 1 (PD-L1). The invention further provides pharmaceutical compositions comprising conjugate and PD-1/PD-L inhibitor.

### BACKGROUND

AXL is a 104-140 kDa transmembrane protein which belongs to the TAM subfamily of mammalian Receptor Tyrosine Kinases (RTKs) and which has transforming abilities (Paccez et al., Int. J. Cancer: 134, 1024-1033 (2013)). The AXL extracellular domain is composed of a combination of two membrane-distal N-terminal immunoglobulin (Ig)-like domains (Ig1 and Ig2 domains) and two membrane-proximal fibronectin type III (FNIII) repeats (the FN1- and FN2-domains) (Paccez et al., 2013; Int. J. Cancer: 134, 1024-1033 (2013)). Enhanced or de novo expression of AXL has been reported in a variety of cancers, including gastric, prostate, ovarian, and lung cancer (Paccez et al., Int. J. Cancer: 134, 1024-1033 (2013)).

AXL can be activated upon binding of its ligand, the vitamin K-dependent growth arrest-specific factor 6 (Gas6). Gas6-binding to AXL leads to AXL dimerization, autophosphorylation and subsequent activation of intracellular signaling pathways, such as the PI3K/AKT, mitogen-activated protein kinase (MAPK), STAT and NE-KB cascades (Leconet et al., Oncogene, 1-10 (2013)). In cancer cells, AXL expression has been associated with tumor cell motility, invasion, migration, and is involved in epithelial-to-mesenchymal transition (EMT) (Linger et al., Expert Opin. Ther. Targets, 14(10):1073-1090 (2010)).

Targeted inhibition of AXL and/or its ligand Gas6 may be effective as anti-tumor therapy using, e.g., small molecules or anti-AXL antibodies (Linger et al., 2010). Anti-AXL antibodies have been described that attenuate NSCLC and breast cancer xenograft growth in vivo by downregulation of receptor expression, reducing tumor cell proliferation and inducing apoptosis (Li et al., Oncogene, 28, 3442-3455 (2009); Ye et al., Oncogene, 1-11 (2010); WO 2011/159980, Genentech). Various other anti-AXL antibodies have also been reported (Leconet et al., 2013; lida et al., Anticancer Research, 34:1821-1828 (2014); WO 2012/175691, INSERM; WO 2012/175692, INSERM; WO 2013/064685, Pierre Fabré Medicaments; WO 2013/090776, INSERM; WO 2009/063965, Chugai Pharmaceuticals and WO 2010/131733), including an ADC based on an anti-AXL antibody and a pyrrolobenzo-diazepine (PBD) dimer (WO 2014/174111, Pierre Fabré Medicament and Spirogen Sarl).

Programmed death 1 (PD-1) is a type I membrane protein of 268 amino acids. PD-1 is a member of the extended CD28/CTLA-4 family of T cell regulators and it is suggested that PD-1 and its ligands negatively regulate immune responses. PD-L1 is the ligand for PD1; it is highly expressed in several cancers and the role of PD-1 in cancer immune evasion is well established. Recently, a number of cancer immunotherapy agents which target the PD-1 and/or PD-L1 have been developed (Sunshine, J. and Taube, J., Curr. Opin. Pharmacol. (2015) 23, 32-38). While anti-PD-1/PD-L1 therapy has been claimed to be among the most effective anti-cancer immunoherapies available, it has been shown that as many as 60% of patients receiving such therapy display primary resistance. Furthermore, the development of acquired resistance in melanoma patients with an objective response to anti-PD-1 therapy has been reported (O'Donnell et al., Genome Medicine (2016) 8:111). Since little is known regarding the mechanisms responsible for resistance in patients receiving anti-PD-1 therapy, few effective therapeutic options are available for such patients.

Hence, there is a need for improved methods of treating cancers which are, or which are predicted to be or become, resistant to treatment with PD-1/PD-L1 inhibitors.

### SUMMARY OF INVENTION

It is an object of the present invention to provide a conjugate of a cytotoxic agent and an antibody capable of binding to human Axl (e.g. human Axl having the sequence set forth in SEQ ID NO: 1) for use in potentiating the therapeutic efficacy or anti-tumor activity of programmed cell death-1 (PD-1) and/or programmed death-ligand 1 (PD-L1) inhibition in a subject, by inducing immunogenic cell death and/or tumor-associated inflammation; e.g. tumor-associated inflammation associated with immunogenic cell death.

The conjugate may be used in combination with an inhibitor of PD-1 and/or PD-L1.

The invention further provides a conjugate of a cytotoxic agent and an antibody capable of binding to human Axl for use in treating cancer in a subject, in combination with an inhibitor of programmed cell death-1 (PD-1) and/or programmed death-ligand 1 (PD-L1). In another aspect, the present invention relates to an inhibitor of PD-1 and/or PD-L1 for use in treating cancer in a subject, in combination with a conjugate of a cytotoxic agent and an antibody or antigen-binding fragment thereof capable of binding to human Axl.

In another aspect, the present invention relates to a method of treating cancer comprising administering to a subject in need thereof
an inhibitor of PD-1 and/or PD-L1; and
a conjugate of a cytotoxic agent and an antibody or antigen-binding fragment thereof capable of binding to human Axl.

Also, the present invention provides a method of potentiating the therapeutic efficacy or anti-tumor activity of programmed cell death-1 (PD-1) and/or programmed death-ligand 1 (PD-L1) inhibition in a subject, by inducing immunogenic cell death and/or tumor-associated inflammation; e.g. tumor-associated inflammation associated with immunogenic cell death.

In yet another aspect, the present invention relates to a pharmaceutical composition or formulation comprising a conjugate of an antibody or antigen-binding fragment thereof capable of binding to human Axl and an inhibitor of PD-1 and/or PD-L1.

Finally, the invention also provides a kit of parts comprising a conjugate of an antibody or antigen-binding fragment thereof capable of binding to human Axl and a programmed cell death-1 (PD-1) pathway inhibitor.

These and other aspects and embodiments are described in more detail in the following sections.

### BRIEF DESCRIPTION OF THE DRAWINGS

FIG. 1: Anti-tumor efficacy of IgG1-AXL-107-vcMMAE in combination with anti-PD-1 (pembrolizumab) in the melanoma cell line xenograft model BLM in the presence of tumor-specific, human T-cells, as described in Example 3. Average tumor size after injection of mice with control T cells or MART-1 T cells, in combination with IgG1-b12-vcMMAE (Ctrl ADC), IgG1-AXL-107-vcMMAE, IgG1-b12-vcMMAE plus anti-PD-1 (pembrolizumab), or IgG1-AXL-107-vcMMAE plus anti-PD-1. Error bars show the standard error of the mean (SEM).

FIG. 2: Kaplan-Meyer graph showing the progression-free survival (tumor size cutoff >500 mm3) of the mice in the different groups in the BLM model, as described in Example 3.

FIG. 3: Anti-tumor efficacy of IgG1-AXL-107-vcMMAE in combination with anti-PD-1 (pembrolizumab) in the NSCLC cell line xenograft model LCLC-103H in the presence of tumor-specific, human T-cells, as described in Example 4. Average tumor size after injection of mice with control T cells or MART-1 T cells, in combination with IgG1-b12-vcMMAE (Ctrl ADC), IgG1-AXL-107-vcMMAE, IgG1-b12-vcMMAE plus anti-PD-1 (pembrolizumab), or IgG1-AXL-107-vcMMAE plus anti-PD-1. Error bars show the standard error of the mean (SEM).

FIG. 4: Kaplan-Meyer graph showing the progression-free survival (tumor size cutoff >500 mm³) of the mice in the different groups in the LCLC-103H model, as described in Example 4.

FIG. 5: IgG1-AXL-107-vcMMAE induces an inflammatory response in patient derived xenograft (PDX) tumors of lung cancer in vivo. (A) Heatmap of the genes differentially modulated by IgG1-AXL-107-vcMMAE treatment (“IgG1-AXL-107-vcMMAE signature”) in PDX tumors treated with IgG1-AXL-107-vcMMAE (4 mg/kg) or Ctrl (IgG1-b12) for 6 days. (B) Gene Set Enrichment Analysis (GSEA) of IgG1-AXL-107-vcMMAE signature, showing significantly enriched gene sets. (C) Proteomics analysis of same tumors used for RNA profiling. Qvalue of <0.1 and fold change of >1.5 were used as cut-off. Green dots indicate significant hits in the interferon response pathways. (D) Growth curves of PDX LXFA526 and LXFA677 in vivo and efficacy of IgG1-AXL-107-vcMMAE. Controls were IgG1-b12MMAE, and naked antibodies of IgG1-b12 or free, unconjugated AXL-107 antibody. Error bars represent SEM. Statistical analysis by Mann-Whitney test; ** p<0.01.

FIG. 6: IgG1-AXL-107-vcMMAE promotes an inflammatory tumor phenotype in an AXL-positive lung cancer PDX model in HIS mice

(A) Heatmap of the IgG1-AXL-107-vcMMAE-associated gene expression signature of the PDX tumors treated with either Ctrl (IgG1-b12) or IgG1-AXL-107-vcMMAE for 3 days or 28-35 days after treatment initiation, termed early time point (TP) and late TP, respectively. (B) Gene set enrichment analyses (GSEA) comparison of Ctrl versus IgG1-AXL-107-vcMMAE-treated tumors, showing significant induction of inflammation-associated gene sets (FDR=false discovery rate). (C) Heatmap of T-cell, NK-cell, and cytotoxic lymphocyte gene expression signatures across all treatment conditions, suggesting enriched prevalence of TILS in IgG1-AXL-107-vcMMAE-treated PDX tumors compared to controls.

FIG. 7: IgG1-AXL-107-vcMMAE induces calreticulin surface expression in vitro. Surface calreticulin expression was measured by flow cytometry as described in Example 7. FIGS. 7A and B show the mean fluorescent intensity (MFI) of the PE-labelled calreticulin detection antibody of LCLC-103H (A) or MDA-MB-231 (B) cells treated with paclitaxel, MMAE (50 or 10 nM), IgG1-AXL-107-vcMMAE, or IgG1-b12-vcMMAE relative to the MFI of untreated cells, which was set to 1 (dotted line). FIGS. 7C and D show the percentage LCLC-103H (C) or MDA-MB-231 (D) cells that are positive for calreticulin upon treatment.

FIG. 8: IgG1-AXL-107-vcMMAE induces extracellular ATP release in vitro. Extracellular ATP release by LCLC-103H and MDA-MB-231 cells treated with paclitaxel, MMAE (50 or 10 nM), IgG1-AXL-107-vcMMAE, or IgG1-b12-vcMMAE was measured as described in Example 7. Relative ATP release from LCLC-103H (A, B) or MDA-MB-231 (C, D) cells under normal (100% PBS; A and C) or hypotonic (70% PBS; B and D) conditions was measured. The ATP release of untreated cells was set to 1 (dotted line).

FIG. 9: IgG1-AXL-107-vcMMAE induces secretion of HMGB1 in vitro. Secretion of HMGB1 by LCLC-103H and MDA-MB-231 cells treated with paclitaxel, MMAE (50 or 10 nM), IgG1-AXL-107-vcMMAE, or IgG1-b12-vcMMAE was measured as described in Example 7. Relative HMGB1 secretion by LCLC-103H (A) or MDA-MB-231 (B) cells was measured. HMGB1 secretion by untreated cells was set to 1 (dotted line).

FIG. 10: IgG1-AXL-107-vcMMAE increases the tumor expression of mouse monocyte/macrophage markers CD14, CD68, and F4/80 in the LXFA-526 (Figures A) and LXFA-677 (Figures B) models 6 days after treatment. The levels of these proteins were measured by proteomic profiling as described in Example 8.

FIG. 11: IgG1-AXL-107-vcMMAE enhances tumor influx of host innate immune cells. Protein expression of the murine macrophage/monocyte marker F4/80 in tumors of the LXFA-526 (Figure A) and LXFA-677 (Figure B) models after treatment with IgG1-AXL-107-vcMMAE (AXL-ADC) or IgG1-b12 (Ab control) for 6 days. The levels of F4/F80 were measured by immunohistochemistry as described in Example 9.

FIG. 12: IgG1-AXL-107-vcMMAE increases the expression of mouse H2 class I (H2-L and B2M) and class II (H2-Aa and H2-Ab1) proteins in the LXFA-526 (Figure A) and LXFA-677 (Figure B) models 6 days after treatment. The levels of these proteins were measured by proteomic profiling as described in Example 8.

FIG. 13: Expression of selected peripheral cytokines (MCP-1, TNFalpha, IL-5) after treatment with IgG1-AXL-107-vcMMAE (AXL-ADC) or IgG1-b12 (Ab control) for 3 days in the LXFA-526 model (Figure A) and in the LXFA-577 model (Figure B). Cytokine levels were measured in plasma as described in Example 8.

FIG. 14: IgG1-AXL-107-vcMMAE promotes an inflammatory tumor phenotype and induces a memory-like state in T cells. (A) GSEA of comparison between IgG1-b12-MMAE versus IgG1-AXL-107-vcMMAE, showing significantly induced inflammation-associated Hallmark gene sets. (B) Mean log value of T memory minus T effector gene sets in mice treated with IgG1-b12-MMAE plus MART-1 T-cells versus IgG1-AXL-107-vcMMAE plus MART-1 T-cells. (C) Expression of TNFRSF9 (CD137) versus PD1 in mice treated with IgG1-b12-MMAE plus MART-1 T-cells versus IgG1-AXL-107-vcMMAE plus MART-1 T-cells. (D) GSEA of comparison between responders (R) versus non-responding (NR) patients for anti-PD-1 immunotherapy in clinical datasets for melanoma. An FDR value of <0.05 was used as cut-off for significance (all depicted gene sets are significant).

### DETAILED DESCRIPTION

The present invention is based on the observation that combination treatment with a conjugate of an Axl antibody and a cytotoxic agent and anti-PD-1 is more efficacious than treatment with the antibody conjugate alone in human xenograft tumor models in the presence of tumor-specific T cells. Inhibition of PD-1 in the context of tumor-specific T cells had no effect on tumor growth and survival, PD-1 inhibition in combination with the antibody conjugate induced potent tumor reduction and survival benefit.

The term “AXL” as used herein, refers to the protein entitled AXL, which is also referred to as UFO or JTK11, a 894 amino acid protein with a molecular weight of 104-140 kDa that is part of the subfamily of mammalian TAM Receptor Tyrosine Kinases (RTKs). The molecular weight is variable due to potential differences in glycosylation of the protein. The AXL protein consists of two extracellular immunoglobulin-like (Ig-like) domains on the N-terminal end of the protein, two membrane-proximal extracellular fibronectin type III (FNIII) domains, a transmembrane domain and an intracellular kinase domain. AXL is activated upon binding of its ligand Gas6, by ligand-independent homophilic interactions between AXL extracellular domains, by autophosphorylation in presence of reactive oxygen species or by transactivation through EGFR, and is aberrantly expressed in several tumor types. In humans, two isoforms of the AXL protein exist, which are produced by alternative splicing: Isoform Long which has the amino acid sequence set forth in SEQ ID NO:130 (UniprotKB P30530-1) and is considered the canonical sequence; and Isoform Short, which differs from Isoform Long in that amino acid residues 429-437 of SEQ ID NO: 130 are missing (UniprotKB P30530-2).

The term “PD-1” when used herein, refers to the human Programmed Death-1 protein, also known as CD279. The amino acid sequence of human PD-1 is provided in SEQ ID NO: 135 (UniProtKB Q15116)

The term “PD-L1” when used herein, refers to the Programmed Death-Ligand 1 protein. PD-L1 is found in humans and other species, and thus, the term “PD-L1” is not limited to human PD-L1 unless contradicted by context. The sequence of human PD-L1 can be found through Genbank accession no. NP_054862.1 and is provided herein as SEQ ID NO: 136.

The term “PD-L2” when used herein, refers to the human Programmed Death 1-Ligand 2 protein. The sequence of human Programmed Death 1-ligand 2 protein precursor may be accessed through Genbank accession no. NP_079515 (SEQ ID NO: 137). Amino acids residues 1-19 are a predicted signal peptide and amino acid residues 20-273 are Programmed Death-ligand 2 protein.

The term “Gas6” when used herein, refers to Growth Arrest-Specific 6. Gas6 functions as a ligand for the TAM family of receptors, including AXL. Gas6 is composed of an N-terminal region containing multiple gamma-carboxyglutamic acid residues (Gla), which are responsible for the specific interaction with the negatively charged phospholipid membrane. Although the Gla domain is not necessary for binding of Gas6 to AXL, it is required for activation of AXL. Gas6 may also be termed as the “ligand to AXL”. Five isoforms of Gas6 are known and the sequences are provided under UniprotKB Q14393-2 (isoform 1), Q4393-1 (Isoform 2), Q14393-3 (Isoform 3), Q14393-4 (Isoform 4) and Q14393-5 (Isoform 5). The sequence of isoform 1 has been chosen as the canonical sequence and is provided herein as SEQ ID NO: 138. Amino acid 1-30 of SEQ ID NO: 138 are a predicted signal peptide and amino acid residues 31-678 are Growth Arrest-Specific 6.

The term “antibody” as used herein is intended to refer to an immunoglobulin molecule, a fragment of an immunoglobulin molecule, or a derivative of either thereof, which has the ability to specifically bind to an antigen under typical physiological and/or tumor-specific conditions with a half-life of significant periods of time, such as at least about 30 minutes, at least about 45 minutes, at least about one hour, at least about two hours, at least about four hours, at least about 8 hours, at least about 12 hours, at least about 24 hours or more, at least about 48 hours or more, at least about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to be internalized). The binding region (or binding domain which may be used herein, both having the same meaning) which interacts with an antigen, comprises variable regions of both the heavy and light chains of the immunoglobulin molecule. The constant regions of the antibodies (Abs) may mediate the binding of the immunoglobulin to host tissues or factors, including various cells of the immune system (such as effector cells) and components of the complement system such as C1q, the first component in the classical pathway of complement activation.

In the context of the present invention, the term “antibody” includes a monoclonal antibody (mAb), an antibody-like polypeptide, such as a chimeric antibody and a humanized antibody, as well as an ‘antibody fragment’ or a ‘fragment thereof’ retaining the ability to specifically bind to the antigen (antigen-binding fragment) provided by any known technique, such as enzymatic cleavage, peptide synthesis, and recombinant techniques, and retaining the ability to be conjugated to a toxin. An antibody as defined according to the invention can possess any isotype unless the disclosure herein is otherwise limited.

As indicated above, the term antibody as used herein, unless otherwise stated or clearly contradicted by context, includes fragments of an antibody that retain the ability to specifically interact, such as bind, to the antigen. It has been shown that the antigen-binding function of an antibody may be performed by fragments of a full-length antibody. Examples of binding fragments encompassed within the term “antibody” include (i) a Fab′ or Fab fragment, a monovalent fragment consisting of the light chain variable domain (VL), heavy chain variable domain (VH), light chain constant region (CL) and heavy chain constant region domain 1 (CH1) domains, or a monovalent antibody as described in WO 2007/059782; (ii) F(ab′)₂ fragments, bivalent fragments comprising two Fab fragments linked by a disulfide bridge at the hinge region; (iii) an Fd fragment consisting essentially of the VH and CH1 domains; (iv) an Fv fragment consisting essentially of the VL and VH domains of a single arm of an antibody, (v) a dAb fragment Ward et al., Nature 341, 544-546 (1989), which consists essentially of a VH domain and is also called domain antibody Holt et al; Trends Biotechnol. 2003 November; 21(11):484-90; (vi) camelid or nanobodies Revets et al; Expert Opin Biol Ther. 2005 January; 5(1):111-24 and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Revets et al; Expert Opin Biol Ther. 2005 January; 5(1):111-24 and Bird et al., Science 242, 423-426 (1988). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention are discussed further herein.

An antibody can be produced in and collected from different in vitro or ex vivo expression or production systems, for example from recombinantly modified host cells, from hybridomas or systems that use cellular extracts supporting in vitro transcription and/or translation of nucleic acid sequences encoding the antibody. It is to be understood that a multitude of different antibodies, the antibodies being as defined in the context of the present invention, is one that can be provided by producing each antibody separately in a production system as mentioned above and thereafter mixing the antibodies, or by producing several antibodies in the same production system.

The term “programmed cell death-1 (PD-1) pathway” or “PD-1 pathway” refers to the molecular signaling pathway comprising cell surface receptor PD-1 and its ligands PD-L1 and PD-L2. Activation of this pathway induces immune tolerance, while inhibition release T-cell suppression, which may lead to immune activation.

“Treatment” or “treating” refers to the administration of an effective amount of a therapeutically active compound of the present invention with the purpose of easing, ameliorating, arresting or eradicating (curing) symptoms or disease states.

An “effective amount” or “therapeutically effective amount” refers to an amount effective, at dosages and for periods of time necessary, to achieve a desired therapeutic result. A therapeutically effective amount of an anti-TF antibody drug conjugate may vary according to factors such as the disease state, age, sex, and weight of the individual, and the ability of the anti-TF antibody drug conjugate to elicit a desired response in the individual. A therapeutically effective amount is also one in which any toxic or detrimental effects of the antibody or antibody portion are outweighed by the therapeutically beneficial effects.

The term “immunoglobulin heavy chain” or “heavy chain of an immunoglobulin” as used herein is intended to refer to one of the heavy chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The term “immunoglobulin” as used herein is intended to refer to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized (see for instance Fundamental Immunology Ch. 7 (Paul, W., ed., 2nd ed. Raven Press, N.Y. (1989)). Within the structure of the immunoglobulin, the two heavy chains are inter-connected via disulfide bonds in the so-called “hinge region”. Equally to the heavy chains, each light chain is typically comprised of several regions; a light chain variable region (abbreviated herein as VL) and a light chain constant region. The light chain constant region typically is comprised of one domain, CL. Furthermore, the VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences are defined according to IMGT (see Lefranc MP. et al., Nucleic Acids Research, 27, 209-212, 1999] and Brochet X. Nucl. Acids Res. 36, W503-508 (2008)).

The term “binding region” or “antigen-binding region” as used herein, refers to a region of an antibody which is capable of binding to the antigen. The antigen can be any molecule, such as a polypeptide, e.g. present on a cell, bacterium, or virion. The terms “antigen” and “target” may, unless contradicted by the context, be used interchangeably in the context of the present invention. The terms “antigen-binding region” and “antigen-binding site” may, unless contradicted by the context, be used interchangeably in the context of the present invention.

The term “bind” or “binding” as used herein refers to the binding of an antibody to, or the ability of an antibody to bind to, a predetermined antigen or target, typically with a binding affinity corresponding to a K_{D} of 1E⁻⁶ M or less, e.g. 5E⁻⁷ M or less, 1E⁻⁷ M or less, such as 5E⁻⁸ M or less, such as 1E⁻⁸ M or less, such as 5E⁻⁹ M or less, such as 1E⁻⁹ M or less, such as 1E⁻¹⁶ M or less, or even 1E⁻¹¹ M or less, when determined by biolayer interferometry using the antibody as the ligand and the antigen as the analyte and binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen.

The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the k_{off} value or off-rate.

The term “kₐ” (M⁻¹×sec⁻¹), as used herein, refers to the association rate constant of a particular antibody-antigen interaction. Said value is also referred to as the kₒₙ value or on-rate.

The term “cytotoxic agent” or “cytostatic” agent is a compound that is detrimental to (e.g., kills) cells. Some cytotoxic or cytostatic moieties for use in ADCs are hydrophobic, meaning that they have no or only a limited solubility in water, e.g., 1 g/L or less (very slightly soluble), such as 0.8 g/L or less, such as 0.6 g/L or less, such as 0.4 g/L or less, such as 0.3 g/L or less, such as 0.2 g/L or less, such as 0.1 g/L or less (practically insoluble). Exemplary hydrophobic cytotoxic or cytostatic moieties include, but are not limited to, certain microtubulin inhibitors such as auristatin and its derivatives, e.g., MMAF and MMAE, as well as maytansine and its derivatives, e.g., DM1.

When used herein in the context of an antibody and a ligand or in the context of two or more antibodies, the term “competes with” indicates that the antibody competes with the ligand or another antibody, e.g., a “reference” antibody in binding to an antigen, respectively. Example 13 of WO 2017/121867 provides an example of how to test competition of an anti-AXL antibody with the AXL-ligand Gas6.

The term “epitope” means an antigenic determinant which is specifically bound by an antibody. Epitopes usually consist of surface groupings of molecules such as amino acids, sugar side chains or a combination thereof and usually have specific three dimensional structural characteristics, as well as specific charge characteristics. Conformational and non-conformational epitopes are distinguished in that the binding to the former but not the latter is lost in the presence of denaturing solvents. The epitope may comprise amino acid residues which are directly involved in the binding, and other amino acid residues, which are not directly involved in the binding, such as amino acid residues which are effectively blocked or covered by the antibody when it is bound to the antigen (in other words, the amino acid residue is within or closely adjacent to the footprint of the specific antibody).

The terms “monoclonal antibody”, or the like, as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be produced by a hybridoma which includes a B cell obtained from a transgenic or transchromosomal non-human animal, such as a transgenic mouse, having a genome comprising a human heavy chain transgene and a light chain transgene, fused to an immortalized cell. Monoclonal antibodies may also be produced from recombinantly modified host cells, or systems that use cellular extracts supporting in vitro transcription and/or translation of nucleic acid sequences encoding the antibody.

The term “isotype” as used herein refers to the immunoglobulin class (for instance IgG1, IgG2, IgG3, IgG4, IgD, IgA, IgE, or IgM) or any allotypes thereof, such as IgG1m(za) and IgG1m(f)) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions. Further, each heavy chain isotype can be combined with either a kappa (κ) (SEQ ID NO: 153) or lambda (λ) light chain (SEQ ID NO: 154).

The term “allotype”, as used herein, refers to the amino acid variation within one isotype class in the same species. The predominant allotype of an antibody isotype varies between ethnicity individuals. The known allotype variations within the IgG1 isotype of the heavy chain result from 4 amino acid substitutions in the antibody frame. In one embodiment the antibody of the invention is of the IgG1m(f) allotype as defined in SEQ ID NO 152. In one embodiment of the invention the antibody of the invention is of the IgG1m(f) allotype as defined in SEQ ID NO 152, wherein at most five amino acid substitutions has been introduced, such as four amino acid substitutions, such as three amino acid substitutions, such as two amino acid substitutions, such as one amino acid substitution.

In the context of the present invention, the term “Ig-like domain I (Ig1) domain” refers in particular to the human Ig-like domain I corresponding to amino acid residues 1-134 in SEQ ID NO: 130 disclosed herein. The Ig-like domain I (Ig1) domain is also termed the “Ig1 domain” herein.

In the context of the present invention, the term “Ig-like domain II (Ig2)” refers in particular to human Ig-like domain II corresponding to amino acid residues 148-194 in SEQ ID NO: 130 disclosed herein. The Ig-like domain II (Ig2)” is also termed the “Ig2 domain” herein.

In the context of the present invention, the term “FNIII-like domain I (FN1)” refers in particular to the human FNIII-like domain I corresponding to amino acid residues 227-329 in SEQ ID NO: 130, also termed “FN1 domain” herein.

In the context of the present invention, the term “FNIII-like domain II (FN2)” refers in particular to the human FNIII-like domain II corresponding to amino acid residues 340-444 in SEQ ID NO: 130, also termed “FN2 domain” herein.

The term “full-length antibody” when used herein, refers to an antibody (e.g., a parent or variant antibody) comprising one or two pairs of heavy and light chains, each containing all heavy and light chain constant and variable domains that are normally found in a heavy chain-light chain pair of a wild-type antibody of that isotype. In a full length variant antibody, the heavy and light chain constant and variable domains may in particular contain amino acid substitutions that improve the functional properties of the antibody when compared to the full length parent or wild type antibody. A full-length antibody according to the present invention may be produced by a method comprising the steps of (i) cloning the CDR sequences into a suitable vector comprising complete heavy chain sequences and complete light chain sequence, and (ii) expressing the complete heavy and light chain sequences in suitable expression systems. It is within the knowledge of the skilled person to produce a full-length antibody when starting out from either CDR sequences or full variable region sequences. Thus, the skilled person would know how to generate a full-length antibody according to the present invention.

The term “human antibody”, as used herein, is intended to include antibodies having variable and framework regions derived from human germline immunoglobulin sequences and a human immunoglobulin constant domain. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “Fc region” as used herein, refers to a region comprising, in the direction from the N- to C-terminal end of the antibody, at least a hinge region, a CH2 region and a CH3 region. An Fc region of the antibody may mediate the binding of the immunoglobulin to host tissues or factors, including various cells of the immune system (such as effector cells) and components of the complement system.

The term “hinge region” as used herein refers to the hinge region of an immunoglobulin heavy chain. Thus, for example the hinge region of a human IgG1 antibody corresponds to amino acids 216-230 according to the Eu numbering as set forth in Kabat (Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example the CH1 region of a human IgG1 antibody corresponds to amino acids 118-215 according to the Eu numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example the CH2 region of a human IgG1 antibody corresponds to amino acids 231-340 according to the Eu numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus for example the CH3 region of a human IgG1 antibody corresponds to amino acids 341-447 according to the Eu numbering as set forth in Kabat (ibid). However, the CH3 region may also be any of the other subtypes as described herein.

The term “full-length” when used in the context of an antibody indicates that the antibody is not a fragment, but contains all of the domains of the particular isotype normally found for that isotype in nature, e.g. the VH, CH1 region, CH2 region, CH3 region, hinge, VL and CL domains for an IgG1 antibody.

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present invention, amino acids may be classified based on structure and chemical characteristics. Thus, classes of amino acids may be reflected in one or both of the following tables:

Main classification based on structure and general chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

Alternative Physical and Functional Classifications of Amino Acid Residues

| Class | Amino acid |
|---|---|
| Hydroxyl group containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, |
| | P, and T |
| Flexible residues | Q, T, K, S, G, P, D, E, and R |

Substitution of one amino acid for another may be classified as a conservative or non-conservative substitution. In the context of the invention, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as thay are both aliphatic, branched hydrophobes. Similarly, aspartic acid may be substituted with glutamic acid since they are both small, negatively charged residues.

For purposes of the present invention, the “sequence identity” between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment).

The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 45%, such as at least about 55%, at least about 65%, at least about 75%, at least about 85%, at least about 90%, at least about 95%, or more (e.g., about 99%) similarity to the parent sequence.

The term “internalized” or “internalization” as used herein, refers to a biological process in which molecules such as the antibody according to the present invention, are engulfed by the cell membrane and drawn into the interior of the cell. Internalization may also be referred to as “endocytosis”.

The term “bystander kill capacity”, “bystander killing effect”, “bystander kill”, or “bystander cytotoxicity” as used herein, refers to the effect where the cytotoxic agent that is conjugated to the antibody by either a cleavable or non-cleavable linker has the capacity to diffuse across cell membranes after the release from the antibody and thereby cause killing of neighboring cells. When the cytotoxic agent is conjugated by a cleavable or non-cleavable linker, it may be either the cytotoxic agent only or the cytotoxic agent with a part of the linker that has the bystander kill capacity. The capacity to diffuse across cell membranes is related to the hydrophobicity of the the cytotoxic agent or the combination of the cytotoxic agent and the linker. Such cytotoxic agents may advantageously be membrane-permeable toxins, such as MMAE that has been released from the antibody by proteases. Especially in tumors with heterogeneous target expression and in solid tumors where antibody penetration may be limited, a bystander killing effect may be desirable.

The term “Drug-to-Antibody Ratio (DAR)” is the average number of cytotoxic agent moieties per antibody in a molecule. The cytotoxic agent loading may occur on amino acids with useful functional groups such as, but not limited to, amino or sulfhydryl groups, as in lysine or cysteine.

Depending on the way of conjugation, DAR may be limited by the number of attachment sites on the antibody, for example where the attachment is a cysteine thiol or a lysine. Generally, antibodies do not contain many free and reactive cysteine thiol groups which may be linked to a drug moiety as most cysteine thiol residues in antibodies exist as disulfide bridges. Therefore, when the cytotoxic agent is conjugated via a cysteine thiol, the antibody may be reduced with reducing agent such as dithiothreitol (DTT) or tricarbonylethylphosphine (TCEP), under partial or fully reducing conditions, to generate reactive cysteine thiol groups.

It is within the capacity of the skilled person to determine the DAR for any given conjugate of an antibody and cytotoxic agent. The DAR may for instance be determined by Liquid Chromatography coupled to Electrospray Ionization Mass Spectrometry (LC-ESI-MS) as described by L. Bassa, “Drug-to-Antibody Ratio (DAR) and Dug Load Distribution by LC-ESI-MS”; in L. Ducry (ed.), Antibody-Drug-Conjugates, Methods in Molecular Biology, Vol 1045, Springer Science+Business Media, LLC 2013, pp. 285-293.

The term “cleavable linker” as used herein, refers to a subset of linkers that are catalyzed by specific proteases in the targeted cell or in the tumor microenvironment, resulting in release of the cytotoxic agent. Examples of cleavable linkers are linkers based on chemical motifs including disulfides, hydrazones or peptides. Another subset of cleavable linker, adds an extra linker motif between the cytotoxic agent and the primary linker, i.e. the site that attaches the linker-drug combination to the antibody. In some embodiments, the extra linker motif is cleavable by a cleavable agent that is present in the intracellular environment (e. g. within a lysosome or endosome or caveola). The linker can be, e. g. a peptidyl linker that is cleaved by an intracellular peptidase or protease enzyme, including but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside the target cells (see e. g. Dubowchik and Walker, 1999, Pharm. Therapeutics 83:67-123). An advantage of using intracellular proteolytic release of the therapeutic agent is that the agent is typically attenuated when conjugated and the serum stabilities of the conjugates are typically high.

The term “non-cleavable linker” as used herein, refers to a subset of linkers which, in contrast to cleavable linkers, do not comprise motifs that are specifically and predictably recognized by intracellular or extracellular proteases. Thus, ADCs based on non-cleavable linkers are not released or cleaved form the antibody until the complete antibody-linker-drug complex is degraded in the lysosomal compartment. Examples of a non-cleavable linker are thioethers. In yet another embodiment, the linker unit is not cleavable and the drug is released by antibody degradation (see US 2005/0238649). Typically, such a linker is not substantially sensitive to the extracellular environment. As used herein, “not substantially sensitive to the extracellular environment” in the context of a linker means that no more than 20%, typically no more than about 15%, more typically no more than about 10%, and even more typically no more than about 5%, no more than about 3%, or no more than about 1% of the linkers, in a sample of antibody drug conjugate compound, are cleaved when the antibody drug conjugate compound is present in an extracellular environment (e.g. plasma). Whether a linker is not substantially sensitive to the extracellular environment can be determined for example by incubating with plasma the antibody drug conjugate compound for a predetermined time period (e.g. 2, 4, 8, 16 or 24 hours) and then quantitating the amount of free drug present in the plasma.

Sequences

TABLE 1

| SEQ ID NO: | Name | Amino acid sequence | Comments |
|---|---|---|---|
| SEQ ID NO: 1 | 107 VH | EVQLLESGGGLVQPGGSLRLSCAASGFTFSSYAMNWVR | HCo12-BalbC |
| | | QAPGKGLEWVSTTSGSGASTYYADSVKGRFTISRDNSK | Ig1 domain |
| | | NTLYLQMNSLRAEDTAVYYCAKIWIAFDIWGQGTMVT | binding Ab |
| | | VSS | |
| SEQ ID NO: 2 | 107 VL | EIVLIQSPGILSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSPYTFGQGTKLEIK | |
| SEQ ID NO: 3 | 140 VH | EVQLLESGGGLVQPGGSLRLSCAASGFTFSSYAMTWVR | |
| | | QAPGKGLEWVSAISISGASTFYADSVKGRFTISRDNSKN | |
| | | TLSLQMNSLRAEDTAVYFCRGYSGYVYDAFDIWGQGT | |
| | | MVTVSS | |
| SEQ ID NO: 4 | 140 VL | DIQMTQSPSSLSASVGDRVTITCRASQGISNWLAWYQ | |
| | | QKPEKAPKSLIYAASSLQSGVPSRFSGSGSGTDFTLTISSL | |
| | | QPEDFATYYCQQYNSYPLTFGGGTKVEIK | |
| SEQ ID NO: 5 | 148 VH | EVQLLESGGGLVQPGGSLRLSCAASGFTFSSYAMTWVR | HCo12-BalbC |
| | | QAPGKGLEWVSAISISGGSTFYADSVKGRFTISRDNSKN | Ig2 domain |
| | | TLYLQMNSLRAEDTAVYYCRGYSGYVYDAFDFWGQGT | binding Ab |
| | | MVTVSS | |
| SEQ ID NO: 6 | 148 VL | DIQMTQSPSSLSASVGDRVTITCRASQGISNWLAWYQ | |
| | | QKPEKAPKSLIYAASSLQSGVPSRFSGSGSGTDFTLTISSL | |
| | | QPEDFATYYCQQYNSYPLTFGGGTKVEIK | |
| SEQ ID NO: 7 | 154 VH | EVQLLDSGGGLVQPGGSLRLSCAASGFTFSSYAMSWVR | HCo12-BalbC |
| | | QAPGKGLEWVSAISIGGGNAYYADSVKGRFTISRDNSK | FN1 domain |
| | | NTLYLQMNSLRAADTAVYYCAKPGFIMVRGPLDYWGQ | binding Ab |
| | | GALVTVSS | |
| SEQ ID NO: 8 | 154-M103L VH | EVQLLDSGGGLVQPGGSLRLSCAASGFTFSSYAMSWVR | |
| | | QAPGKGLEWVSAISIGGGNAYYADSVKGRFTISRDNSK | |
| | | NTLYLQMNSLRAADTAVYYCAKPGFILVRGPLDYWGQ | |
| | | GALVTVSS | |
| SEQ ID NO: 9 | 154 VL | EIVLIQSPGILSLSPGERATLSCRASQSVSNSYLAWYQQ | |
| | | KPGQAPRLLIYGASSRATGIPDRFSGSGSGTDFTLTISRLE | |
| | | PEDFAVYYCQQYGSSPYTFGQGTKLEIK | |
| SEQ ID NO: 10 | 171 VH | EVQLLESGGGLVQPGGSLRLSCAASGFTFSSYAMSWVR | HCo17-BalbC |
| | | QAPGKGLEWVSDISVSGGSTYYADSVKGRFTISRDNSK | Ig2 domain |
| | | NTLYLQMNSLRAEDTAVYYCAKEGYIWFGESLSYAFDI | binding Ab |
| | | WGQGTMVTVSS | |
| SEQ ID NO: 11 | 171 VL | EIVLIQSPGILSLSPGERATLSCRASQSVSSSYLAWYQQK | |
| | | PGQAPRLLIYGASSRATGIPDRFSGSGSGTDFTLTISRLEP | |
| | | EDFAVYYCQQYGRSFTFGPGTKVDIK | |
| SEQ ID NO: 12 | 172 VH | EVQLLESGGGLVQPGGSLRLSCAASGFTFSNYAMSWVR | |
| | | QAPGKGLEWVSDISVSGGSTYYADSVKGRFTISRDNSK | |
| | | NTLYLQMNSLRAEDTAVYYCAKEGYIWFGESLSYAFDI | |
| | | WGQGTMVTVSS | |
| SEQ ID NO: 13 | 172 VL | EIVLIQSPGILSLSPGERATLSCRASQSVSSSYLAWYQQK | |
| | | PGQAPRLLIYGASSRATGIPDRFSGSGSGTDFTLTISRLEP | |
| | | EDFAVYYCQQYGRSFTFGPGTKVDIK | |
| SEQ ID NO: 14 | 181 VH | EVQLLESGGGLVQPGGSLRLSCAASGFTFSSYAMSWVR | |
| | | QAPGKGLEWVSDISVSGGSTYYADSVKRFTISRDNSK | |
| | | NTLYLHMNSLRAEDTAVYYCAKEGYIWFGESLSYAFDIW | |
| | | GQGTMVTVSS | |
| SEQ ID NO: 15 | 181 VH | EIVLIQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGRSFTFGPGTKVDIK | |
| SEQ ID NO: 16 | 183 VH | QVQLQQWGALLKPSETLSLTCAVYGGSFSGYYWSWI | HCo17-BalbC |
| | | RQPPGKGLEWIGEINQSGSTNYNPSLKSRVTISVDTSKN | FN1 domain |
| | | QFSLKLSSVTAADTSVYYCASGNWDHFFDYWGQGTLV | binding Ab |
| | | TVSS | |
| SEQ ID NO: 17 | 183-N52Q VH | QVQLQQWGALLKPSETLSLTCAVYGGSFSGYYWSWI | |
| | | RQPPGKGLEWIGEIQQSGSTNYNPSLKSRVTISVDTSKN | |
| | | QFSLKLSSVTAADTSVYYCASGNWDHFFDYWGQGTLV | |
| | | TVSS | |
| SEQ ID NO: 18 | 183 VL | DIQMTQSPSSVSASVDRVTITCRASQGISSWLAWYQH | |
| | | KPGKAPKLLIYATSSLQSGVTSRFSGSGSGTDFTLTISSLQ | |
| | | PEDFATYYCQQAKSFPWTFGQGTKVEIK | |
| SEQ ID NO: 19 | 187 VH | QVPLQQWGALLKPSETLSLTCAVYGGSFSGYHWSWI | |
| | | RQPPGKGLEWIGEISHSGRTNYNPSLKSRVTISIDTSKNQ | |
| | | FSLKLSSVTAADTAVYYCASFITMIRGTIITHFDYWGQGT | |
| | | LVTVSS | |
| SEQ ID NO: 20 | 187 VL | DIQMTQSPSSLSASVDRVTITCRASQGISSWLAWYQQ | |
| | | KPEKAPKSLIYAASSLQSGVPSRFSGSGSGTDFTLTISSLQ | |
| | | PEDFATYYCQQYHSYPYTFGQGTKLEIK | |
| SEQ ID NO: 21 | 608-01 VH | QVQLVQSGAEVKKPGSSVKVSCKASGGTFSSYAISWVR | |
| | | QAPGQLEWMGRIIPIFGIANYVQKFQGRVTITADKSTS | |
| | | TAYMELSSLRAEDTAVYYCARRDYYGSGSPDVFDIWG | |
| | | QGTMVTVSS | |
| SEQ ID NO: 22 | 608-01 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSYTFGQGTKLEIK | |
| SEQ ID NO: 23 | 610-01 VH | QVQLVQSGAEVKKPGSSVKVSCKASGGTFSSYAISWVR | |
| | | QAPGQLEWMGRIIPIFGIANYVQKFQGRVTITADKSTS | |
| | | TAYMELSSLRAEDTAVYYCARRNYYGSGSPDVFDIWG | |
| | | QGTMVTVSS | |
| SEQ ID NO: 24 | 610-01 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSYTFGQGTKLEIK | |
| SEQ ID NO: 25 | 613 VH | QVQLVQSGAEVKKPGSSVKVSCKASGGTFSSYAINWM | HCo20 |
| | | RQAPQGLEWMGRIIPIFGIVNYAQKFQGRVTLTADKS | Ig1 domain |
| | | TSTAYMELSSLRSEDTAVYYCARRGNYYGSGSPDVFDIW | binding Ab |
| | | GQGTMVTVSS | |
| SEQ ID NO: 26 | 613 VL | EIVLIQSPGILSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSYTFGQGTKLEIK | |
| SEQ ID NO: 27 | 613-08 VH | QVQLVQSGAEVKKPGSSVKVSCKASGGTFSSYAINWM | |
| | | RQAPQGLEWMGRIIPIFGIVNYAQKFQGRVTLTADKS | |
| | | TSTAYMELSSLRSEDTAVYYCARRGNYYGSGSPDVFDIW | |
| | | GQGTMVTVSS | |
| SEQ ID NO: 28 | 613-08 VL | EIVLTQSPATLSLSPGERATLSCRASQSVSSYLAWYQQKP | |
| | | GQAPRLLIYDASNRATGIPARFSGSGSGTDFTLTISSLEPE | |
| | | DFAVYYCQQRSNWLTFGGGTKVEIK | |
| SEQ ID NO: 29 | 620-06 VH | QVQLVQSGAEVKKPGSSVKVSCKASGGTFSSYAISWVR | |
| | | QAPGQLEWMGRIIPIFGIANYAQKFQGRVTITADKSTS | |
| | | TAYMELSSLRSEDTAVYYCARRNYYGSGSPDVFDIWG | |
| | | QGTMVTVSS | |
| SEQ ID NO: 30 | 620-06 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSYTFGQGTKLEIK | |
| SEQ ID NO: 31 | 726 VH | QVQLQQWGALLKPSETLSLTCAIDGGSFSGYYWSWIR | HCo17-BalbC |
| | | QPPGKGLEWIGEISHSGRTNYNPSLKSRVTISIDTSKNQF | FN2 domain |
| | | SLKLSSVAAADTAVYYCARFITMIRGAIITHFDYWGQGA | binding Ab |
| | | LVTVSS | |
| SEQ ID NO: 32 | 726-M 101L VH | QVQLQQWGALLKPSETLSLICAIDGGSFSGYYWSWIR | |
| | | QPPGKGLEWIGEISHSGRTNYNPSLKSRVTISIDTSKNQF | |
| | | SLKLSSVAAADTAVYYCARFITLIRGAIITHFDYWGQGAL | |
| | | VTVSS | |
| SEQ ID NO: 33 | 726 VL | DIQMTQSPSSLSASVDRVTITCRASQGISSWLAWYQQ | |
| | | KPEKAPKSLIYAASSLQSGVPSRFSGSGSGTDFTLTISSLQ | |
| | | PEDFATYYCQQYHSYPYTFGQGTKLEIK | |
| SEQ ID NO: 34 | 733 VH | QVQLVESGGGVVQPGRSLRLSCAASGFSFSTYAMHWV | HCo17-BalbC |
| | | RQAPGKGLEWVAVISYDGDNKYSADSVKGRFTISRDNS | FN1 domain |
| | | KNTLYLQMNSLRAEDTAVYYCARGRKLGIDAFDIWGQG | binding Ab |
| | | TMVTVSS | |
| SEQ ID NO: 35 | 733 VL | AIQLTQSPSSLSASVGDRVTITCRASQGISSALAWYQQK | |
| | | PGKAPKLLIYDASSLESGVPSRFSGSGSGTDFTLTISGLQP | |
| | | EDFATYYCQQFNSYPFTFGPGTKVDIK | |
| SEQ ID NO: 36 | 107 VH CDR1 | GFTFSSYA | |
| SEQ ID NO: 37 | 107 VH CDR2 | TSGSGAST | |
| SEQ ID NO: 38 | 107 VH CDR3 | AKIWIAFDI | |
| SEQ ID NO: 39 | 107 VL CDR1 | QSVSSSY | |
| | 107 VL CDR2 | GAS | |
| SEQ ID NO: 40 | 107 VL CDR3 | QQYGSSPYT | |
| SEQ ID NO: 41 | 140 VH CDR1 | GFTFSSYA | |
| SEQ ID NO: 42 | 140 VH CDR2 | ISISGAST | |
| SEQ ID NO: 43 | 140 VH CDR3 | RGYSGYVYDAFDI | |
| SEQ ID NO: 44 | 140 VL CDR1 | QGISNW | |
| | 140 VL CDR2 | AAS | |
| SEQ ID NO: 45 | 140 VL CDR3 | QQYNSYPLT | |
| SEQ ID NO: 46 | 148 VH CDR1 | GFTFSSYA | |
| SEQ ID NO: 47 | 148 VH CDR2 | ISISGGST | |
| SEQ ID NO: 48 | 148 VH CDR3 | RGYSGYVYDAFDF | |
| SEQ ID NO: 49 | 148 VL CDR1 | QGISNW | |
| | 148 VL CDR2 | AAS | |
| SEQ ID NO: 50 | 148 VL CDR3 | QQYNSYPLT | |
| SEQ ID NO: 51 | 154 VH CDR1 | GFTFSSYA | |
| SEQ ID NO: 52 | 154 VH CDR2 | ISIGGGNA | |
| SEQ ID NO: 53 | 154 VH CDR3 | AKPGFIMVRGPLDY | |
| SEQ ID NO: 54 | 154-M103L VH | AKPGFILVRGPLDY | |
| | CDR3 | | |
| SEQ ID NO: 55 | 154 VL CDR1 | QSVSNSY | |
| | 154 VL CDR2 | GAS | |
| SEQ ID NO: 56 | 154 VL CDR3 | QQYGSSPYT | |
| SEQ ID NO: 57 | 171 VH CDR1 | GFTFSSYA | |
| SEQ ID NO: 58 | 171 VH CDR2 | ISVSGGST | |
| SEQ ID NO: 59 | 171 VH CDR3 | AKEGYIWFGESLSYAFDI | |
| SEQ ID NO: 60 | 171 VL CDR1 | QSVSSSY | |
| | 171 VL CDR2 | GAS | |
| SEQ ID NO: 61 | 171 VL CDR3 | QQYGRSFT | |
| SEQ ID NO: 62 | 172 VH CDR1 | GFTFSNYA | |
| SEQ ID NO: 63 | 172 VH CDR2 | ISVSGGST | |
| SEQ ID NO: 64 | 172 VH CDR3 | AKEGYIWFGESLSYAFDI | |
| SEQ ID NO: 65 | 172 VL CDR1 | QSVSSSY | |
| | 172 VL CDR2 | GAS | |
| SEQ ID NO: 66 | 172 VL CDR3 | QQYGRSFT | |
| SEQ ID NO: 67 | 181 VH CDR1 | GFTFSSYA | |
| SEQ ID NO: 68 | 181 VH CDR2 | ISVSGGST | |
| SEQ ID NO: 69 | 181 VH CDR3 | AKEGYIWFGESLSYAFDI | |
| SEQ ID NO: 70 | 181 VL CDR1 | QSVSSSY | |
| | 181 VL CDR2 | GAS | |
| SEQ ID NO: 71 | 181 VL CDR3 | QQYGRSFT | |
| SEQ ID NO: 72 | 183 VH CDR1 | GGSFSGYY | |
| SEQ ID NO: 73 | 183 VH CDR2 | INQSGST | |
| SEQ ID NO: 74 | 183-N52Q VH CDR2 | IQQSGST | |
| SEQ ID NO: 75 | 183 VH CDR3 | ASGNWDHFFDY | |
| SEQ ID NO: 76 | 183 VL CDR1 | QGISSW | |
| | 183 VL CDR2 | ATS | |
| SEQ ID NO: 77 | 183 VL CDR3 | QQAKSFPWT | |
| SEQ ID NO: 78 | 187 VH CDR1 | GGSFSGYH | |
| SEQ ID NO: 79 | 187 VH CDR2 | ISHSGRT | |
| SEQ ID NO: 80 | 187 VH CDR3 | ASFITMIRGTIITHFDY | |
| SEQ ID NO: 81 | 187 VL CDR1 | QGISSW | |
| | 187 VL CDR2 | AAS | |
| SEQ ID NO: 82 | 187 VL CDR3 | QQYHSYPYT | |
| SEQ ID NO: 83 | 608-01 VH CDR1 | GGTFSSYA | |
| SEQ ID NO: 84 | 608-01 VH CDR2 | IIPIFGIA | |
| SEQ ID NO: 85 | 608-01 VH CDR3 | ARRGDYYGSGSPDVFDI | |
| SEQ ID NO: 86 | 608-01 VL CDR1 | QSVSSSY | |
| | 608-01 VL CDR2 | GAS | |
| SEQ ID NO: 87 | 608-01 VL CDR3 | QQYGSSYT | |
| SEQ ID NO: 88 | 610-01 VH CDR1 | GGTFSSYA | |
| SEQ ID NO: 89 | 610-01 VH CDR2 | IIPIFGIA | |
| SEQ ID NO: 90 | 610-01 VH CDR3 | ARRGNYYGSGSPDVFDI | |
| SEQ ID NO: 91 | 610-01 VL CDR1 | QSVSSSY | |
| | 610-01 VL CDR2 | GAS | |
| SEQ ID NO: 92 | 610-01 VL CDR3 | QQYGSSYT | |
| SEQ ID NO: 93 | 613 VH CDR1 | GGTFSSYA | |
| SEQ ID NO: 94 | 613 VH CDR2 | IIPIFGIV | |
| SEQ ID NO: 95 | 613 VH CDR3 | ARRGNYYGSGSPDVFDI | |
| SEQ ID NO: 96 | 613 VL CDR1 | QSVSSSY | |
| | 613 VL CDR2 | GAS | |
| SEQ ID NO: 97 | 613 VL CDR3 | QQYGSSYT | |
| SEQ ID NO: 98 | 613-08 VH CDR1 | GGTFSSYA | |
| SEQ ID NO: 99 | 613-08 VH CDR2 | IIPIFGIV | |
| SEQ ID NO: 100 | 613-08 VH CDR3 | ARRGNYYGSGSPDVFDI | |
| SEQ ID NO: 101 | 613-08 VL CDR1 | QSVSSY | |
| | 613-08 VL CDR2 | DAS | |
| SEQ ID NO: 102 | 613-08 VL CDR3 | QQRSNWLT | |
| SEQ ID NO: 103 | 620-06 VH CDR1 | GGTFSSYA | |
| SEQ ID NO: 104 | 620-06 VH CDR2 | IIPIFGIA | |
| SEQ ID NO: 105 | 620-06 VH CDR3 | ARRGNYYGSGSPDVFDI | |
| SEQ ID NO: 106 | 620-06 VL CDR1 | QSVSSSY | |
| | 620-06 VL CDR2 | GAS | |
| SEQ ID NO: 107 | 620-06 VL CDR3 | QQYGSSYT | |
| SEQ ID NO: 108 | 726 VH CDR1 | GGSFSGYY | |
| SEQ ID NO: 109 | 726 VH CDR2 | ISHSGRT | |
| SEQ ID NO: 110 | 726 VH CDR3 | ARFITMIRGAIITHFDY | |
| SEQ ID NO: 111 | 726-M101L VH | ARFITLIRGAIITHFDY | |
| | CDR3 | | |
| SEQ ID NO: 112 | 726 VL CDR1 | QGISSW | |
| | 726 VL CDR2 | AAS | |
| SEQ ID NO: 113 | 726 VL CDR3 | QQYHSYPYT | |
| SEQ ID NO: 114 | 733 VH CDR1 | GFSFSTYA | |
| SEQ ID NO: 115 | 733 VH CDR2 | ISYDGDNK | |
| SEQ ID NO: 116 | 733 VH CDR3 | ARGRKLGIDAFDI | |
| SEQ ID NO: 117 | 733 VL CDR1 | QGISSA | |
| | 733 VL CDR2 | DAS | |
| SEQ ID NO: 118 | 733 VL CDR3 | QQFNSYPFT | |
| SEQ ID NO: 119 | Ig2 domain VH | ISISGXST-wherein X is A or G | |
| | CDR2 | | |
| SEQ ID NO: 120 | Ig2 domain VH | RGYSGYVYDAFDX-wherein X is I or F | |
| | CDR3 | | |
| SEQ ID NO: 121 | FN2 domain VH | GGSFSGYX-wherein X is H or Y | |
| | CDR1 | | |
| SEQ ID NO: 122 | FN2 domain VH | AX1FITMIRGX2IITHFDY-wherein X1 is S or R; and | |
| | CDR3 | X2 is T or A | |
| SEQ ID NO: 123 | FN1 domain VH | GFTFSXYA-wherein X is S or N | |
| | CDR1 | | |
| SEQ ID NO: 124 | FN1 domain VH | ISVSGGST | |
| | CDR2 | | |
| SEQ ID NO: 125 | FN1 domain VH | AKEGYIWFGESLSYAFDI | |
| | CDR3 | | |
| SEQ ID NO: 126 | Ig1 domain VH | IIPIFGIX-wherein X is A or V | |
| | CDR2 | | |
| SEQ ID NO: 127 | Ig1 domain VH | ARRGXYYGSGSPDVFDI-wherein X is D or N | |
| | CDR3 | | |
| SEQ ID NO: 128 | Ig1 domain VL | QSVXSSY-wherein X is S or del | |
| | CDR1 | | |
| | Ig1 domain VL | XAS-wherein X is D or G | |
| | CDR2 | | |
| SEQ ID NO: 129 | Ig1 domain VL | QQX1X2X3X4X5T-wherein X1 is R or Y; X2 is S or | |
| | CDR3 | G; X3 is N or S; X4 is W or S; and X5 is L or Y | |
| SEQ ID NO: 130 | Human AXL protein | MAWRCPRMGRVPLAWCLALCGWACMAPRGTQAEES | |
| | (Swissprot P30530) | PFVGNPGNITGARGLIGTLRCQLQVQGEPPEVHWLRD | |
| | | GQILELADSTQTQVPLGEDEQDDWIVVSQLRITSLQLSD | |
| | | TGQYQCLVFLHQTFVSQPGYVGLEGLPYFLEEPEDRTV | |
| | | AANTPFNLSCQAQPPEPVDLLWLQDAVPLATAPH | |
| | | PQRSLHVPLNKTSSFSCEAHNAKGVTTSRTATITVLPQ | |
| | | QPRNLHLVSRQPTELEVAWTPGLSGIYPLTHCTLQAVLS | |
| | | DDGMGIQAGEPDPPEEPLTSQASVPPHQLRLGSLHPHT | |
| | | PYHIRVACTSSQGPSSWTHWLPVETPEGVPLGPPENISA | |
| | | TRNGSQAFVHWQEPRAPLQGTLLGYRLAYQGQDTPEV | |
| | | LMDIGLRQEVTLELQGDGSVSNLTVCVAAYTAAGDGP | |
| | | WSLPVPLEAWRPGQAQPVHQLVKEPSTPAFSWPWWY | |
| | | VLLGAVVAAACVLILALFLVHRRKKETRYGEVFEPTVER | |
| | | ELVVRYRVRKSYSRRTTEATLNSLGISEELKEKLRDVMVD | |
| | | RHKVALGKTLGEGEFGAVMEGQLNQDDSILKVAVKTM | |
| | | KIAICTRSELEDFLSEAVCMKEFDHPNVMRLIGVCFQGS | |
| | | ERESFPAPVVILPFMKHGDLHSFLLYSRLGDQPVYLPTQ | |
| | | MLVKFMADIASGMEYLSTKRFIHRDLAARNCMLNENM | |
| | | SVCVADFGLSKKIYNGDYYRQGRIAKMPVKWIAIESLAD | |
| | | RVYTSKSDVWSFGVTMWEIATRGQTPYPGVENSEIYDY | |
| | | LRQGNRLKQPADCLDGLYALMSRCWELNPQDRPSFTE | |
| | | LREDLENTLKALPPAQEPDEILYVNMDEGGGYPEPPGA | |
| | | AGGADPPTQPDPKDSCSCLTAAEVHPAGRYVLCPSTTP | |
| | | SPAQPADRGSPAAPGQEDGA | |
| SEQ ID NO: 131 | Homo sapiens AXL- | MAWRCPRMGRVPLAWCLALCGWACMAPRGTQAEES | |
| | Mus musculus Ig1 | PFVGNPGNITGARGLIGTLRCQLQVQGEPPEVHWLRD | |
| | domain | GQILELADSTQTQVPLGEDEQDDWIVVSQLRITSLQLSD | |
| | | TGQYQCLVFLHQTFVSQPGYVGLEGLPYFLEEPEDKAV | |
| | | PANTPFNLSCQAQGPPEPVTLLWLQDAVPLAPVTGHSS | |
| | | QHSLQTPGLNKTSSFSCEAHNAKGVTTSRTATITVLPQQ | |
| | | PRNLHLVSRQPTELEVAWTPGLSGIYPLTHCTLQAVLSD | |
| | | DGMGIQAGEPDPPEEPLTSQASVPPHQLRLGSLHPHTP | |
| | | YHIRVACTSSQGPSSWTHWLPVETPEGVPLGPPENISAT | |
| | | RNGSQAFVHWQEPRAPLOGILLGYRLAYQGQDTPEVL | |
| | | MDIGLRQEVTLELQGDGSVSNLTVCVAAYTAAGDGPW | |
| | | SLPVPLEAWRPGQAQPVHQLVKEPSTPAFSWPWWYV | |
| | | LLGAVVAAACVLILALFLVHRRKKETRYGEVFEPTVERGE | |
| | | LVVRYRVRKSYSRRTTEATLNSLGISEELKEKLRDVMVDR | |
| | | HKVALGKTLGEGEFGAVMEGQLNQDDS | |
| | | ILKVAVKTMKIAICTRSELEDFLSEAVCMKEFDHPNVMR | |
| | | LIGVCFQGSERESFPAPVVILPFMKHGDLHSFLLYSRLGD | |
| | | QPVYLPTQMLVKFMADIASGMEYLSTKRFIHRDLAARN | |
| | | CMLNENMSVCVADFGLSKKIYNGDYYRQGRIAKMPVK | |
| | | WIAIESLADRVYTSKSDVWSFGVTMWEIATRGQTPYP | |
| | | VENSEIYDYLRQGNRLKQPADCLDGLYALMSRCWELNP | |
| | | QDRPSFTELREDLENTLKALPPAQEPDEILYVNMDEG | |
| | | GYPEPPGAAGGADPPTQPDPKDSCSCLTAAEVHPAGRY | |
| | | VLCPSTTPSPAQPADRGSPAAPGQEDGA | |
| SEQ ID NO: 132 | Homo sapiens AXL- | MAWRCPRMGRVPLAWCLALCGWACMAPRGTQAEES | |
| | Mus musculus Ig2 | PFVGNPGNITGARGLIGTLRCQLQVQGEPPEVHWLRD | |
| | domain | GQILELADSTQTQVPLGEDEQDDWIVVSQLRITSLQLSD | |
| | | TGQYQCLVFLGHQTFVSQPGYVGLEGLPYFLEEPEDKAV | |
| | | PANTPFNLSCQAQGPPEPVTLLWLQDAVPLAPVTGHSS | |
| | | QHSLQTPGLNKTSSFSCEAHNAKGVTTSRTATITVLPQQ | |
| | | PRNLHLVSRQPTELEVAWTPGLSGIYPLTHCTLQAVLSD | |
| | | DGMGIQAGEPDPPEEPLTSQASVPPHQLRLGSLHPHTP | |
| | | YHIRVACTSSQGPSSWTHWLPVETPEGVPLGPPENISAT | |
| | | RNGSQAFVHWQEPRAPLOGILLGYRLAYQGQDTPEVL | |
| | | MDIGLRQEVTLELQGDGSVSNLTVCVAAYTAAGDGPW | |
| | | SLPVPLEAWRPGQAQPVHQLVKEPSTPAFSWPWWYV | |
| | | LLGAVVAAACVLILALFLVHRRKKETRYGEVFEPTVERGE | |
| | | LVVRYRVRKSYSRRTTEATLNSLGISEELKEKLRDVMVDR | |
| | | HKVALGKTLGEGEFGAVMEGQLNQDDS | |
| | | ILKVAVKTMKIAICTRSELEDFLSEAVCMKEFDHPNVMR | |
| | | LIGVCFQGSERESFPAPVVILPFMKHGDLHSFLLYSRLGD | |
| | | QPVYLPTQMLVKFMADIASGMEYLSTKRFIHRDLAARN | |
| | | CMLNENMSVCVADFGLSKKIYNGDYYRQGRIAKMPVK | |
| | | WIAIESLADRVYTSKSDVWSFGVTMWEIATRGQTPYP | |
| | | VENSEIYDYLRQGNRLKQPADCLDGLYALMSRCWELNP | |
| | | QDRPSFTELREDLENTLKALPPAQEPDEILYVNMDEG | |
| | | GYPEPPGAAGGADPPTQPDPKDSCSCLTAAEVHPAGRY | |
| | | VLCPSTTPSPAQPADRGSPAAPGQEDGA | |
| SEQ ID NO: 133 | Homo sapiens AXL- | MAWRCPRMGRVPLAWCLALCGWACMAPRGTQAEES | |
| | Mus musculus | PFVGNPGNITGARGLIGTLRCQLQVQGEPPEVHWLRD | |
| | FN1 domain | GQILELADSTQTQVPLGEDEQDDWIVVSQLRITSLQLSD | |
| | | TGQYQCLVFLHQTFVSQPGYVGLEGLPYFLEEPEDRTV | |
| | | AANTPFNLSCQAQPPEPVDLLWLQDAVPLATAPH | |
| | | PQRSLHVPLNKTSSFSCEAHNAKGVTTSRTATITVLPQ | |
| | | RPHHLHVVSRQPTELEVAWTPGLSGIYPLTHCNLQAVLS | |
| | | DDGVIWLGKSDPPEDPLTLQVSVPPHQLRLEKLLPHTP | |
| | | YHIRISCSSSQGPSPWTHWLPVETTEGVPLGPPENISAT | |
| | | RNGSQAFVHWQEPRAPLQUILLGYRLAYQGQDTPEVL | |
| | | MDIGLRQEVTLELQDGSVSNLTVCVAAYTAAGDGPW | |
| | | SLPVPLEAWRPGQAQPVHQLVKEPSTPAFSWPWWYV | |
| | | LLGAVVAAACVLILALFLVHRRKKETRYEVFEPTVERGE | |
| | | LVVRYRVRKSYSRRTTEATLNSLGISEELKEKLRDVMVDR | |
| | | HKVALGKTLGEGEFGAVMEGQLNQDDS | |
| | | ILKVAVKTMKIAICTRSELEDFLSEAVCMKEFDHPNVMR | |
| | | LIGVCFQGSERESFPAPVVILPFMKHGDLHSFLLYSRLGD | |
| | | QPVYLPTQMLVKFMADIASGMEYLSTKRFIHRDLAARN | |
| | | CMLNENMSVCVADFGLSKKIYNGDYYRQGRIAKMPVK | |
| | | WIAIESLADRVYTSKSDVWSFGVTMWEIATRGQTPYP | |
| | | VENSEIYDYLRQGNRLKQPADCLDGLYALMSRCWELNP | |
| | | QDRPSFTELREDLENTLKALPPAQEPDElLYVNMDEG | |
| | | GYPEPPGAAGGADPPTQPDPKDSCSCLTAAEVHPAGRY | |
| | | VLCPSTTPSPAQPADRGSPAAPGQEDGA | |
| SEQ ID NO: 134 | Homo sapiens AXL- | MAWRCPRMGRVPLAWCLALCGWACMAPRGTQAEES | |
| | Mus musculus | PFVGNPGNITGARGLIGTLRCQLQVQGEPPEVHWLRD | |
| | FN2 domain | GQILELADSTQTQVPLGEDEQDDWIVVSQLRITSLQLSD | |
| | | TGQYQCLVFLGHQTFVSQPGYVGLEGLPYFLEEPEDRTV | |
| | | AANTPFNLSCQAQPPEPVDLLWLQDAVPLATAPH | |
| | | PQRSLHVPLNKTSSFSCEAHNAKGVTTSRTATITVLPQ | |
| | | QPRNLHLVSRQPTELEVAWTPGLSGIYPLTHCTLQAVLS | |
| | | DDGMIQAGEPDPPEEPLTSQASVPPHQLRLGSLHPHT | |
| | | PYHIRVACTSSQGPSSWTHWLPVETPEGVPLGPPENVS | |
| | | AMRNGSQVLVRWQEPRVPLQGTLLGYRLAYRGQDTPE | |
| | | VLMDIGLTREVTLELRGDRPVANLTVSVTAYTSAGDGP | |
| | | WSLPVPLEPWRPGQGQPLHHLVSEPPPRAFSWPWWY | |
| | | VLLGAVVAAACVLILALFLVHRRKKETRYGEVFEPTVER | |
| | | ELVVRYRVRKSYSRRTTEATLNSLGISEELKEKLRDVMVD | |
| | | RHKVALGKTLGEGEFGAVMEGQLNQDDSILKVAVKTM | |
| | | KIAICTRSELEDFLSEAVCMKEFDHPNVMRLIGVCFQGS | |
| | | ERESFPAPVVILPFMKHGDLHSFLLYSRLGDQPVYLPTQ | |
| | | MLVKFMADIASGMEYLSTKRFIHRDLAARNCMLNENM | |
| | | SVCVADFGLSKKIYNGDYYRQGRIAKMPVKWIAIESLAD | |
| | | RVYTSKSDVWSFGVTMWEIATRGQTPYPGVENSEIYDY | |
| | | LRQGNRLKQPADCLDGLYALMSRCWELNPQDRPSFTE | |
| | | LREDLENTLKALPPAQEPDEILYVNMDEGGGYPEPPGA | |
| | | AGGADPPTQPDPKDSCSCLTAAEVHPAGRYVLCPSTTP | |
| | | SPAQPADRGSPAAPGQEDGA | |
| SEQ ID NO: 135 | Programmed cell | MQ1PQAPWPVVWAVLQLGWRPGWELDSPDRPWNP | |
| | death protein 1 | PTFSPALLVVTEGDNATFTCSFSNTSESFVLNWYRMSPS | |
| | (UniProtKB | NQTDKLAAFPEDRSQPGQDCRFRVTQLPNGRDFHMSV | |
| | Q15116) | VRARRNDSGTYLCGAISLAPKAQIKESLRAELRVTERRAE | |
| | | VPTAHPSPSPRPAGQFQTLVVGVVGGLLGSLVLLVWVL | |
| | | AVICSRAARGTIGARRTGQPLKEDPSAVPVFSVDYGELD | |
| | | FQWREKTPEPPVPCVPEQTEYATIVFPSGMGTSSPARR | |
| | | GSADGPRSAQPLRPEDGHCSWPL | |
| SEQ ID NO: 136 | PD-L1 | MRIFAVFIFMTYWHLLNAFTVTVPKDLYVVEYGSNMTI | |
| | (Genbank accession | ECKFPVEKQLDLAALIVYWEMEDKNIIQFVHGEEDLKVQ | |
| | no. NP_054862.1) | HSSYRQRARLLKDQLSLGNAALQITDVKLQDAGVYRCM | |
| | | ISYGGADYKRITVKVNAPYNKINQRILVVDPVTSEHELTC | |
| | | QAEGYPKAEVIWTSSDHQVLSGKITTINSKREEKLENVT | |
| | | STLRINTTTNEIFYCTFRRLDPEENHTAELVIPELPLAHPP | |
| | | NERTHLVILGAILLCLGVALTFIFRLRKGRMMDVKKCGIQ | |
| | | DTNSKKQSDTHLEET | |
| SEQ ID NO: 137 | Programmed Death | MIFLLLMLSLELQLHQIAALFTVTVPKELYIIEHGSNVTLE | |
| | 1-ligand 2 protein | CNFDTGSHVNLGAITASLQKVENDTSPHRERATLLEEQL | |
| | precursor (Genbank | PLGKASFHIPQVQVRDEGQYQCIIIYGVAWDYKYLTLKV | |
| | accession no. | KASYRKINTHILKVPETDEVELTCQATGYPLAEVSWPNV | |
| | NP_079515) | SVPANTSHSRTPELYQVTSVLRLKPPPGRNFSCVFWN | |
| | | THVRELTLASIDLQSQMEPRTHPTWLLHIFIPFCIIAFIFIA | |
| | | TVIALRKQLCQKLYSSKDTTKRPVTTTKREVNSAI | |
| SEQ ID NO: 138 | Growth Arrest- | MAPSLSPGPAALRRAPQLLLLLLAAECALAALLPAREAT | |
| | Specific 6 precursor | QFLRPRQRRAFQVFEEAKQGHLERECVEELCSREEAREV | |
| | (Genbank accession | FENDPETDYFYPRYLDCINKYGSPYTKNSGFATCVQNLP | |
| | no. NP_000811) | DQCTPNPCDRKGTQACQDLMGNFFCLCKAGWGGRLC | |
| | | DKDVNECSQENGGCLQICHNKPGSFHCSCHSGFELSSD | |
| | | GRTCQDIDECADSEACGEARCKNLPGSYSCLCDEGFAYS | |
| | | SQEKACRDVDECLQGRCEQVCVNSPGSYTCHCDGRG | |
| | | LKLSQDMDTCEDILPCVPFSVAKSVKSLYLGRMFSGTPVI | |
| | | RLRFKRLQPTRLVAEFDFRTFDPEGILLFAGGHQDSTWI | |
| | | VLALRAGRLELQLRYNGVGRVTSSGPVINHGMWQTISV | |
| | | EELARNLVIKVNRDAVMKIAVAGDLFQPERGLYHLNLTV | |
| | | GGIPFHEKDLVQPINPRLDGCMRSWNWLNGEDTTIQE | |
| | | TVKVNTRMQCFSVTERGSFYPGSGFAFYSLDYMRTPLD | |
| | | VGTESTWEVEVVAHIRPAADTGVLFALWAPDLRAVPLS | |
| | | VALVYHSTKKLKKQLVVLAVEHTALALMEIKVCDGQEH | |
| | | VVTVSLRDGEATLEVDGTRGQSEVSAAQLQERLAVLER | |
| | | HLRSPVLTFAGGLPDVPVTSAPVTAFYRGCMTLEVNRRL | |
| | | LDLDEAAYKHSDITAHSCPPVEPAAA | |
| SEQ ID NO: 139 | SEQ ID NO: 1 of | MGFKMESQFQVFVFVFLWLSGVDGEVQLVESGGDLVK | |
| | WO2016166296 | PGGSLKLSCAASGFTFSSYGMSWVRQTPDKRLEWVATI | |
| | | SSGGSYTYYPDSVKGRFTISRDNAKNTLYLQMSSLKSED | |
| | | TAMYYCARHPIYYTYDDTMDYWGQGTSVTVSS | |
| SEQ ID NO: 140 | SEQ ID NO: 4 of | MGFKMESQFQVFVFVFLWLSGVDGENVLTQSPAIMAA | |
| | WO2016166296 | SPGEKVTTCSASSSVSSGNFHWYQQKPGTSPKLWIYRTS | |
| | | NLASGVPARFSGSGSGTSYSLTISSMEAEDAATYYCQQ | |
| | | WSGYPWTFGGGTKLEIK | |
| SEQ ID NO: 141 | SEQ ID NO: 2 of | MGFKMESQFQVFVFVFLWLSGVDGQVQLVESGGGW | |
| | WO2016166296 | QPGRSLRLSCAASGFTFSSYGMSVRQAPGKGLEWVATI | |
| | | SSGGSYTYYPDSVKGRFTISRDNSKNTLYLQMNSLRAED | |
| | | TAVYYCARHPIYYTYDDTMDYWGQGTTVTVSS | |
| SEQ ID NO: 142 | SEQ ID NO: 5 of | MGFKMESQFQVFVFVFLWLSGVDGEIVLTQSPATLSLS | |
| | WO2016166296 | PGERATLSCSASSSVSSGNFHWYQQKPGLAPRLLIYRTS | |
| | | NLASGIPDRFSGSGSGTDFTLTISRLEPEDFAVYYCQQW | |
| | | SGYPWTFGPGTKVDIK | |
| SEQ ID NO: 143 | SEQ ID NO: 7 of | MGFKMESQFQVFVFVFLWLSGVDGEIVLTQSPGTLSLS | |
| | WO2016166296 | PGERATLSCSASSSVSSGNFHWYQQKPGLAPRLLIYRTS | |
| | | NLASGIPARFSGSGSGTDFTLT1SSLEPEDFAVYYCQQW | |
| | | SGYPWTFGGGTKLEIK | |
| SEQ ID NO: 144 | SEQ ID NO: 3 of | MGFKMESQFQVFVFVFLWLSGVDGEVQLVESGGGLV | |
| | WO2016166296 | QPGGSLRLSCAASGFTFSSYGMSWVRQAPGKGLEWVA | |
| | | TISSGGSYTYYPDSVKGRFTISRDNAKNSLYLQMNSLRAE | |
| | | DTAVYYCARHPIYYTYDDTMDYWGQGTLVTVSS | |
| SEQ ID NO: 145 | SEQ ID NO: 11 of | gaggtccagctggtacagtctggggctgaggtgaagaagcctggggc | |
| | WO2017180842 | tacagtgaaaatctcctgcaaggtttctggttactcattcactggcgcta | |
| | | ccatgaactggatccgccagcccccagggaaggggctggagtggatt | |
| | | ggtcttattaaaccttccaatggtggtactagttacaaccagaagttca | |
| | | agggcagagtcaccatctcagccgacaagtccatcagcaccgcctac | |
| | | ctgcagtggagcagcctgaaggcctcggacaccgccatgtattactgt | |
| | | gcacatggtcactacgagagttacgaggctatggactactggggcca | |
| | | gggaacgctggtcaccgtcagctca | |
| SEQ ID NO: 146 | SEQ ID NO: 7 of | gacatccagatgacccagtctccatcctccctgtctgcatctgtaggag | |
| | WO2017180842 | acagagtcaccatcacttgcaaggccagtcaggatgtggtttctgctgt | |
| | | agcctggtaccagcagaaacctggccaggctcccaggctcctcatcta | |
| | | ttggcaggatacccggcacactggagtcccatcaaggttcagcggca | |
| | | gtggatctgggacagaattcactctcaccatcagcagcctgcagcctg | |
| | | atgattttgcaacttattactgtcaggaacattttagcactccgctcac | |
| | | gttcggccaagggaccaaggtggaaatcaaacc | |
| SEQ ID NO: 147 | SEQ ID NO: 8 of | gacatccagatgacccagtctccatcctccctgtctgcatctgtaggag | |
| | WO2017180842 | acagagtcaccatcacttgcaaggccagtcaggatgtgagttctgctg | |
| | | tagcctggtaccagcagaaacctggccaggctcccaggctcctcatct | |
| | | attggcaggatacccggcacactggagtcccatcaaggttcagcggc | |
| | | agtggatctgggacagaattcactctcaccatcagcagcctgcagcct | |
| | | gatgattttgcaacttattactgtcaggaacattttagccctccgctcac | |
| | | gttcggccaagggaccaaggtggaaatcaaacc | |
| SEQ ID NO: 148 | SEQ ID NO: 9 of | gacatccagatgacccagtctccatcctccctgtctgcatctgtaggag | |
| | WO2017180842 | acagagtcaccatcacttgcaaggccagtcaggatgtggtttctgctgt | |
| | | agcctggtaccagcagaaacctggccaggctcccaggctcctcatcta | |
| | | ttggcaggatacccggcacactggagtcccatcaaggttcagcggca | |
| | | gtggatctgggacagaattcactctcaccatcagcagcctgcagcct | |
| | | gatgattttgcaacttattactgtcaggaacattttagccctccgctcac | |
| | | gttcggccaagggaccaaggtggaaatcaaacc | |
| SEQ ID NO: 149 | SEQ ID NO: 10 of | gacatccagatgacccagtctccatcctccctgtctgcatctgtaggag | |
| | WO2017180842 | acagagtcaccatcacttgcaaggccagtcaggatgtggtttctgctgt | |
| | | agcctggtaccagcagaaacctggccaggctcccaggctcctcatcta | |
| | | ttggcaggatacccggcacactggagtcccatcaaggttcagcggca | |
| | | gtggatctgggacagaattcactctcaccatcagcagcctgcagcct | |
| | | gatgattttgcaacttattactgtcaggaacattttagccctccgctcag | |
| | | gttcggccaagggaccaaggtggaaatcaaacc | |
| SEQ ID NO: 150 | SEQ ID NO: 12 of | gaggtccagctggtacagtctggggctgaggtgaagaagcctggggc | |
| | WO2017180842 | tacagtgaaaatctcctgcaaggtttctggttactcattctggggcgct | |
| | | accatgaactggatccgccagcccccagggaaggggctggagtggat | |
| | | tggtcttattaaaccttccaatggtggtactagttacaaccagaagttc | |
| | | aagggcagagtcaccatctcagccgacaagtccatcagcaccgccta | |
| | | cctgcagtggagcagcctgaaggcctcggacaccgccatgtattactg | |
| | | tgcacatggtcactacgagagttacgaggctatggactactggggcca | |
| | | gggaacgctggtcaccgtcagctca | |
| SEQ ID NO: 151 | SEQ ID NO: 13 of | gaggtccagctggtacagtctggggctgaggtgaagaagcctggggc | |
| | WO2017180842 | tacagtgaaaatctcctgcaaggtttctggttactcattcactggccac | |
| | | accatgaactggatccgccagcccccagggaaggggctggagtggat | |
| | | tggtcttattaaaccttccaatggtggtactagttacaaccagaagt | |
| | | tcaagggcagagtcaccatctcagccgacaagtccatcagcaccgcc | |
| | | tacctgcagtggagcagcctgaaggcctcggacaccgccatgtattac | |
| | | tgtgcacatggtcactacgagagttacgaggctatggactactggggc | |
| | | cagggaacgctggtcaccgtcagctca | |
| SEQ ID NO: 152 | Fc IgG1m (f) | ASTKGPSVFPLAPSSKSTSGGTAALGCLVKDYFPEPVTVS | |
| | aa 118-447 | WNSGALTSGVHTFPAVLQSSGLYSLSSVVTVPSSSLGTQ | |
| | EU numbering | TYICNVNHKPSNTKVDKRVEPKSCDKTHTCPPCPAPELL | |
| | | GGPSVFLFPPKPKDTLMISRTPEVTCVVVDVSHEDPEVK | |
| | | FNWYVDGVEVHNAKTKPREEQYNSTYRVVSVLTVLHQ | |
| | | DWLNGKEYKCKVSNKALPAPIEKTISKAKGQPREPQVYT | |
| | | LPPSREEMTKNQVSLTCLVKGFYPSDIAVEWESNGQPE | |
| | | NNYKTTPPVLDSDGSFFLYSKLTVDKSRWQQGNVFSCS | |
| | | VMHEALHNHYTQKSLSLSPGK | |
| SEQ ID NO: 153 | kappa (κ) light | RTVAAPSVFIFPPSDEQLKSGTASVVCLLNNFYPREAKV | |
| | chain | QWKVDNALQSGNSQESVTEQDSKDSTYSLSSTLTLSKA | |
| | | DYEKHKVYACEVTHQGLSSPVTKSFNRGEC | |
| SEQ ID NO: 154 | lambda (λ) light | GQPKAAPSVTLFPPSSEELQANKATLVCLISDFYPGAVTV | |
| | chain | AWKADSSPVKAGVETTTPSKQSNNKYAASSYLSLTPEQ | |
| | | WKSHRSYSCQVTHEGSTVEKTVAPTECS | |

In a first aspect, the present invention provides a conjugate of a cytotoxic agent and an antibody capable of binding to human Axl (e.g. human Axl having the sequence set forth in SEQ ID NO: 1) for use in potentiating the therapeutic efficacy or anti-tumor activity of of programmed cell death-1 (PD-1) and/or programmed death-ligand 1 (PD-L1) inhibition in a subject, such as a subject suffering from cancer and/or carrying a tumor, by inducing immunogenic cell death and/or tumor-associated inflammation; e.g. tumor-associated inflammation associated with immunogenic cell death.

The use may further be to potentiate the clinical efficacy of anti PD-1 and/or anti PD-L1 therapy provided to said subject.

The conjugate may be used according to the invention, in combination with an inhibitor of PD-1 and/or PD-L1.

The invention further provides a conjugate of a cytotoxic agent and an antibody capable of binding to human Axl (e.g. human Axl having the sequence set forth in SEQ ID NO: 1) for use in treating cancer in a subject, in combination with an inhibitor of programmed cell death-1 (PD-1) and/or programmed death-ligand 1 (PD-L1).

Preferably, the conjugate is of a cytotoxic agent and an antibody, wherein the antibody does not compete for AXL binding with the ligand Growth Arrest-Specific 6 (Gas6).

Competition between anti-AXL and the ligand Gas6 to AXL may be determined as described in Example 2 of WO 2016/005593, under the heading “Competition between AXL antibodies and Gas6 for AXL binding”. Hence, the ability of the antibody to compete for AXL binding with the ligand Gas6 may be determined in an assay comprising the steps of:
i) incubating AXL-expressing cells, such as A431 cells, with Gas6,
ii) adding anti-AXL antibodies to be tested,
iii) adding a fluorescently labelled secondary reagent detecting anti-AXL antibodies and
iv) analyzing the cells by FACS.

Alternatively, the antibody does not compete for binding with the ligand Gas6, wherein the competing for binding is determined in an assay comprising the steps of:
i) incubating AXL-expressing cells with anti-AXL antibodies,
ii) adding Gas6,
iii) adding a fluorescently labelled secondary reagent detecting Gas6, and
iv) analyzing the cells by FACS.

In particular, the maximal binding of the antibody to AXL in the presence of Gas6 may be at least 90%, such as at least 95%, such as at least 97%, such as at least 99%, such as 100%, of binding in the absence of Gas6 as determined by a competition assay, wherein competition between said antibody or antigen-binding fragment and Gas6 is determined on A431 cells preincubated with Gas6 and without Gas6, such as in one of the assays disclosed above.

The antibody may have a binding affinity (KD) in the range of 0.3×10⁻⁹ to 63×10⁻⁹ M to human AXL, The binding affinity may be measured using Bio-layer Interferometry using soluble AXL extracellular domain. In particular, the binding affinity may be determined by a method comprising the steps of;
i) loading anti-human Fc Capture biosensors with anti-AXL antibodies, and
ii) determining association and dissociation of soluble recombinant AXL extracellular domain by Bio-Layer Interferometry at different concentrations.

A soluble recombinant AXL extracellular domain as used herein is preferably an AXL extracellular domain that has been expressed recombinantly. Due to absence of the transmembrane and intracellular domain, recombinant AXL extracellular domain is not attached to a, e.g. cell surface and stays in solution. It is well-known how to express a protein recombinantly, and thus, it is within the knowledge of the skilled person to provide such recombinant AXL extracellular domain.

The antibody may in particular have a dissociation rate of 9.7×10⁻⁵ to 4.4×10⁻³ s⁻¹ to AXL; preferably wherein the dissociation rate is measured by Bio-layer Interferometry using soluble recombinant AXL extracellular domain. The dissociation rate may in particular be measured by a method comprising the steps of
i) loading anti-human Fc Capture biosensors with anti-AXL antibodies, and
ii) determining association and dissociation of recombinant AXL extracellular domain by Bio-Layer Interferometry at different concentrations.

The term “dissociation rate” as used herein, refers to the rate at which an antigen-specific antibody bound to its antigen, dissociates from that antigen, and is expressed as s⁻¹. Thus, in the context of an antibody binding AXL, the term “dissociation rate”, refers to the antibody binding AXL dissociates from the recombinant extracellular domain of AXL, and is expressed as s⁻¹.

Preferably, the antibody is capable of being internalized when cell surface bound.

The ability of the antibody to be internalization may be determined by a procedure comprising the steps of:
i) Seeding cells, such as MDA-MB-231 or Calu-1 cells (human lung carcinoma cell line; ATCC, catalognumber HTB-54), in 96-well tissue culture plates, 50.000 cells per well, and allowing the cells to attach for 6 hrs at 37° C.,
ii) Incubating the cells in tissue culture medium with serial dilutions of AXL-antibodies (final concentration range 0.0032-10 μg/mL) at 4° C. for 1 hour,
iii) Replacing the tissue culture medium with antibody by tissue culture medium without antibody and incubating the cells overnight (16-18 hours) at 37° C. or 4° C.
iv) Detaching the cells with 40 μL warm trypsin solution, and washing the cells with ice-cold PBS/0.1% BSA/0.02% azide, and incubating the cells for 30 minutes at 4° C. with R-Phycoerythrin (PE)-conjugated goat-anti-human IgG F(ab′)2 (Jackson ImmunoResearch Laboratories, Inc., West Grove, Pa.; cat. No. 109-116-098) diluted 1/100 in PBS/0.1% BSA/0.02% azide (final volume 100 μL), washing the cells were twice in PBS/0.1% BSA/0.02% azide, resuspending the cells in 120 μL PBS/0.1% BSA/0.02% azide,
v) Analyzing the cells by flow cytometry, wherein binding curves are analyzed using non-linear regression (sigmoidal dose-response with variable slope), optionally using GraphPad Prism V5.04 or later software (GraphPad Software, San Diego, Calif., USA).

In certain embodiments of the invention, the conjugate comprises an antibody, wherein
a) the antibody binds to an epitope within the Ig-like domain I (Ig1) domain of human Axl,
b) the antibody binds to an epitope which comprises or requires one or more amino acids corresponding to positions L121 to Q129 or T112 to Q124 of human AXL having the sequence set forth in SEQ ID NO: 1, and/or
c) binding of the antibody to a chimeric Axl molecule as set forth in SEQ ID NO: 131 is reduced, such as by at least 50% compared to binding of the antibody to human Axl having the sequence set forth in SEQ ID NO: 1, when binding is determined as described in Example 3 of WO 2016/005593.

In other embodiments the conjugate comprises an antibody, wherein
a) the antibody binds to an epitope within the Ig-like domain II (Ig2) of human AXL,
b) the antibody binds to an epitope which comprises or requires the amino acids corresponding to position D170 to R190 or the combination of D179 and one or more amino acids corresponding to positions T182 to R190 of human AXL having the sequence set forth in SEQ ID NO: 1, and/or
c) binding of the antibody to a chimeric Axl molecule as set forth in SEQ ID NO: 132 is reduced, such as by at least 50% compared to binding of the antibody to human Axl having the sequence set forth in SEQ ID NO: 1, when binding is determined as described in Example 3 of WO 2016/005593.

Alternatively, the conjugate comprises an antibody, wherein
a) the antibody binds to an epitope within the FNIII-like domain I (FN1) of human AXL,
b) the antibody binds to an epitope, which comprises or requires one or more amino acids corresponding to positions Q272 to A287 and G297 to P301 of human AXL of human AXL having the sequence set forth in SEQ ID NO: 1, and/or
c) binding of the antibody to a chimeric Axl molecule as set forth in SEQ ID NO: 133 is reduced, such as by at least 50% compared to binding of the antibody to human Axl having the sequence set forth in SEQ ID NO: 1, when binding is determined as described in Example 3 of WO 2016/005593.

In still further embodiments, the conjugate comprises an antibody, wherein
a) the antibody binds to an epitope within the FNIII-like domain II (FN2) of human AXL,
b) the antibody binds to an epitope which comprises or requires the amino acids corresponding to positions A359, R386, and one or more amino acids corresponding to positions Q436 to K439 of human AXL having the sequence set forth in SEQ ID NO: 1, and/or
c) binding of the antibody to a chimeric Axl molecule as set forth in SEQ ID NO: 134 is reduced, such as by at least 50% compared to binding of the antibody to human Axl having the sequence set forth in SEQ ID NO: 1, when binding is determined as described in Example 3 of WO 2016/005593.

Mapping of the the epitope or the region within Axl to which the antibodies bind may be achieved by determining the binding of the antibodies to human Axl and/or chimeric Axl, by a procedure comprising the steps of
I) providing cells, such as HEK293T cells, transiently transfected with an expression construct for human Axl having the sequence set forth in SEQ ID NO: 130, or an expression construct for a chimeric Axl molecule as set forth in any one of SEQ ID NOs: 131 to 134;
II) incubating the cells with serial dilutions of said antibody with a final concentration range of 0.0024-10 μg/mL for 30 minutes at 4° C.;
III) washing three times in PBS/0.1% BSA/0.02% azide, incubating the cells with R-Phycoerythrin (PE)-conjugated goat-anti-human IgG F(ab′)2 diluted 1/100 in PBS/0.1% BSA/0.02% azide, washing the cells twice in PBS/0.1% BSA/0.02% azide, and resuspending the cells in 120 μL PBS/0.1% BSA/0.02% azide; and
IV) analyzing the cells by flow cytometry.

The conjugate that is used according to the invention may comprise an antibody, which comprises at least one binding region comprising a variable heavy chain (VH) region and a variable light chain (VL) region selected from the group consisting of:
a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148];
c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733];
d) a VH region comprising the CDR1, CDR2, and CDR3 sequences of SEQ ID Nos.: 51, 52, and 53, respectively; and a VL region comprising the CDR1, CDR2, and CDR3 sequences of SEQ ID Nos.: 55, GAS, and 56, respectively [154];
e) a VH region comprising the CDR1, CDR2, and CDR3 sequences of SEQ ID Nos.: 51, 52, and 54, respectively; and a VL region comprising the CDR1, CDR2, and CDR3 sequences of SEQ ID Nos.: 55, GAS, and 56, respectively [154-M103L];
f) a VH region comprising the CDR1, CDR2, and CDR3 sequences of SEQ ID Nos.: 57, 58, and 59, respectively; and a VL region comprising the CDR1, CDR2, and CDR3 sequences of SEQ ID Nos.: 60, GAS, and 61, respectively, [171];
g) a VH region comprising the CDR1, CDR2, and CDR3 sequences of SEQ ID Nos.: 62, 63, and 64, respectively; and a VL region comprising the CDR1, CDR2, and CDR3 sequences of SEQ ID Nos.: 65, GAS, and 66, respectively, [172];
h) a VH region comprising the CDR1, CDR2, and CDR3 sequences of SEQ ID Nos.: 67, 68, and 69, respectively; and a VL region comprising the CDR1, CDR2, and CDR3 sequences of SEQ ID Nos.: 70, GAS, and 71, respectively, [181]; (SEQ ID Nos.: 67, 68, 69, 70, 71 of P83)
i) a VH region comprising the CDR1, CDR2, and CDR3 sequences of SEQ ID Nos.: 72, 73, and 75, respectively; and a VL region comprising the CDR1, CDR2, and CDR3 sequences of SEQ ID Nos.: 76, ATS, and 77, respectively, [183];
j) a VH region comprising the CDR1, CDR2, and CDR3 sequences of SEQ ID Nos.: 72, 74, and 75, respectively; and a VL region comprising the CDR1, CDR2, and CDR3 sequences of SEQ ID Nos.: 76, ATS, and 77, respectively, [183-N52Q];
k) a VH region comprising the CDR1, CDR2, and CDR3 sequences of SEQ ID Nos.: 78, 79, and 80, respectively; and a VL region comprising the CDR1, CDR2, and CDR3 sequences of SEQ ID Nos.: 81, AAS, and 82, respectively, [187];
l) a VH region comprising the CDR1, CDR2, and CDR3 sequences of SEQ ID Nos.: 83, 84, and 85, respectively; and a VL region comprising the CDR1, CDR2, and CDR3 sequences of SEQ ID Nos.: 86, GAS, and 87, respectively, [608-01];
m) a VH region comprising the CDR1, CDR2, and CDR3 sequences of SEQ ID Nos.: 88, 89, and 90, respectively; and a VL region comprising the CDR1, CDR2, and CDR3 sequences of SEQ ID Nos.: 9, GAS, and 92, respectively, [610-01];
n) a VH region comprising the CDR1, CDR2, and CDR3 sequences of SEQ ID Nos.: 93, 94, and 95, respectively; and a VL region comprising the CDR1, CDR2, and CDR3 sequences of SEQ ID Nos.: 96, GAS, and 97, respectively, [613];
o) a VH region comprising the CDR1, CDR2, and CDR3 sequences of SEQ ID Nos.: 98, 99, and 100, respectively; and a VL region comprising the CDR1, CDR2, and CDR3 sequences of SEQ ID Nos.: 101, DAS, and 102, respectively, [613-08];
p) a VH region comprising the CDR1, CDR2, and CDR3 sequences of SEQ ID Nos.: 103, 104, and 105, respectively; and a VL region comprising the CDR1, CDR2, and CDR3 sequences of SEQ ID Nos.: 106, GAS, and 107, respectively, [620-06];
q) a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively, [726];
r) a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively, [726-M101L];
s) a VH region comprising the CDR1, CDR2, and CDR3 sequences of SEQ ID Nos.: 41, 42, and 43, respectively; and a VL region comprising the CDR1, CDR2, and CDR3 sequences of SEQ ID Nos.: 44, AAS, and 45, respectively, [140];
t) a VH region comprising the CDR1, CDR2, and CDR3 sequences of SEQ ID Nos.: 93, 94, and 95, respectively, and a VL region comprising the CDR1, CDR2, and CDR3 sequences of SEQ ID Nos.: 128, XAS, wherein X is D or G, and 129, respectively, [613/613-08];
u) a VH region comprising the CDR1, CDR2, and CDR3 sequences of SEQ ID Nos.: 46, 119, and 120, respectively; and a VL region comprising CDR1, CDR2, and CDR3 sequences of SEQ ID Nos.: 49, AAS, and 50, respectively, [148/140];
v) a VH region comprising the CDR1, CDR2, and CDR3 sequences of SEQ ID Nos.: 123, 124, and 125, respectively; and a VL region comprising CDR1, CDR2, and CDR3 sequences of SEQ ID Nos.: 60, GAS, and 61, respectively [171/172/181];
w) a VH region comprising the CDR1, CDR2, and CDR3 sequences of SEQ ID Nos.: 121, 109, and 122, respectively; and a VL region comprising the CDR1, CDR2, and CDR3 sequences of SEQ ID Nos.: 112, AAS, and 113, respectively [726/187]; and
x) a VH region comprising the CDR1, CDR2, and CDR3 sequences of SEQ ID Nos.: 93, 126, and 127, respectively; and a VL region comprising the CDR1, CDR2, and CDR3 sequences of SEQ ID Nos.: 96, GAS, and 97, respectively [613/608-01/610-01/620-06].

The conjugate used according to the invention may in particular comprise an antibody that comprises at least one binding region comprising a variable heavy chain (VH) region and a variable light chain (VL) region selected from the group consisting of:
a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
b) a VH region comprising the CDR1, CDR2, and CDR3 sequences of SEQ ID Nos.: 93, 94, and 95, respectively; and a VL region comprising the CDR1, CDR2, and CDR3 sequences of SEQ ID Nos.: 96, GAS, and 97, respectively, [613];
c) a VH region comprising the CDR1, CDR2, and CDR3 sequences of SEQ ID Nos.: 98, 99, and 100, respectively; and a VL region comprising the CDR1, CDR2, and CDR3 sequences of SEQ ID Nos.: 101, DAS, and 02, respectively, [613-08];
d) a VH region comprising the CDR1, CDR2, and CDR3 sequences of SEQ ID Nos.: 93, 94, and 95, respectively, and a VL region comprising the CDR1, CDR2, and CDR3 sequences of SEQ ID Nos.: 128, XAS, wherein X is D or G, and 129, respectively, [613/613-08]; and
e) a VH region comprising the CDR1, CDR2, and CDR3 sequences of SEQ ID Nos.: 93, 126, and 127, respectively; and a VL region comprising the CDR1, CDR2, and CDR3 sequences of SEQ ID Nos.: 96, GAS, and 97, respectively [613/608-01/610-01/620-06].

The antibody may in particular comprise at least one binding region comprising a variable heavy chain (VH) region and a variable light chain (VL) region selected from the group consisting of:
a) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148]; and
b) a VH region comprising the CDR1, CDR2, and CDR3 sequences of SEQ ID Nos.: 57, 58, and 59, respectively; and a VL region comprising the CDR1, CDR2, and CDR3 sequences of SEQ ID Nos.: 60, GAS, and 61, respectively, [171].

In other embodiments the antibody comprises at least one binding region comprising a variable heavy chain (VH) region and a variable light chain (VL) region selected from the group consisting of:
a) a VH region comprising the CDR1, CDR2, and CDR3 sequences of SEQ ID Nos.: 51, 52, and 53, respectively; and a VL region comprising the CDR1, CDR2, and CDR3 sequences of SEQ ID Nos.: 55, GAS, and 56, respectively [154];
b) a VH region comprising the CDR1, CDR2, and CDR3 sequences of SEQ ID Nos.: 72, 73, and 75, respectively; and a VL region comprising the CDR1, CDR2, and CDR3 sequences of SEQ ID Nos.: 76, ATS, and 77, respectively, [183]; and
c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733].

The antibody may comprise a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively [726].

The conjugate preferably comprises an antibody that comprises at least one binding region comprising a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively [107].

The at least one binding region of the antibody in the conjugate used according to the invention may comprise a VH region and a VL region selected from the group consisting of;
a) a VH region comprising an amino acid sequence having at least 90%, at least 95%, at least 97%, at least 99%, or 100% sequence identity to SEQ ID No: 1 and a VL region comprising an amino acid sequence having at least 90%, at least 95%, at least 97%, at least 99%, or 100% sequence identity to SEQ ID No: 2 [107];
b) a VH region comprising an amino acid sequence having at least 90%, at least 95%, at least 97%, at least 99%, or 100% sequence identity to SEQ ID No: 5 and a VL region comprising an amino acid sequence having at least 90%, at least 95%, at least 97%, at least 99%, or 100% sequence identity to SEQ ID No: 6 [148];
c) a VH region comprising an amino acid sequence having at least 90%, at least 95%, at least 97%, at least 99%, or 100% sequence identity to SEQ ID No: 34 and a VL region comprising an amino acid sequence having at least 90%, at least 95%, at least 97%, at least 99%, or 100% sequence identity to SEQ ID No: 35 [733];
d) a VH region comprising an amino acid sequence having at least 90%, at least 95%, at least 97%, at least 99%, or 100% sequence identity to SEQ ID No: 7 and a VL region comprising an amino acid sequence having at least 90%, at least 95%, at least 97%, at least 99%, or 100% sequence identity to SEQ ID No: 9 [154];
e) a VH region comprising an amino acid sequence having at least 90%, at least 95%, at least 97%, at least 99%, or 100% sequence identity to SEQ ID No: 10 and a VL region comprising an amino acid sequence having at least 90%, at least 95%, at least 97%, at least 99%, or 100% sequence identity to SEQ ID No: 11 [171];
f) a VH region comprising an amino acid sequence having at least 90%, at least 95%, at least 97%, at least 99%, or 100% sequence identity to SEQ ID No: 16 and a VL region comprising an amino acid sequence having at least 90%, at least 95%, at least 97%, at least 99%, or 100% sequence identity to SEQ ID No: 18 [183];
g) a VH region comprising an amino acid sequence having at least 90%, at least 95%, at least 97%, at least 99%, or 100% sequence identity to SEQ ID No: 25 and a VL region comprising an amino acid sequence having at least 90%, at least 95%, at least 97%, at least 99%, or 100% sequence identity to SEQ ID No: 26 [613];
h) a VH region comprising an amino acid sequence having at least 90%, at least 95%, at least 97%, at least 99%, or 100% sequence identity to SEQ ID No: 31 and a VL region comprising an amino acid sequence having at least 90%, at least 95%, at least 97%, at least 99%, or 100% sequence identity to SEQ ID No: 33 [726];
i) a VH region comprising an amino acid sequence having at least 90%, at least 95%, at least 97%, at least 99%, or 100% sequence identity to SEQ ID No: 3 and a VL region comprising an amino acid sequence having at least 90%, at least 95%, at least 97%, at least 99%, or 100% sequence identity to SEQ ID No: 4 [140];
j) a VH region comprising an amino acid sequence having at least 90%, at least 95%, at least 97%, at least 99%, or 100% sequence identity to SEQ ID No: 8 and a VL region comprising an amino acid sequence having at least 90%, at least 95%, at least 97%, at least 99%, or 100% sequence identity to SEQ ID No: 9 [154-M103L];
k) a VH region comprising an amino acid sequence having at least 90%, at least 95%, at least 97%, at least 99%, or 100% sequence identity to an amino acid sequence having at least 90%, at least 95%, at least 97%, at least 99%, or 100% sequence identity to SEQ ID No: 12 and a VL region comprising an amino acid sequence having at least 90%, at least 95%, at least 97%, at least 99%, or 100% sequence identity to SEQ ID No: 13 [172] (;
l) a VH region comprising an amino acid sequence having at least 90%, at least 95%, at least 97%, at least 99%, or 100% sequence identity to SEQ ID No: 14 and a VL region comprising an amino acid sequence having at least 90%, at least 95%, at least 97%, at least 99%, or 100% sequence identity to SEQ ID No: 15 [181];
m) a VH region comprising an amino acid sequence having at least 90%, at least 95%, at least 97%, at least 99%, or 100% sequence identity to SEQ ID No: 17 and a VL region comprising an amino acid sequence having at least 90%, at least 95%, at least 97%, at least 99%, or 100% sequence identity to SEQ ID No: 18 [183-N52Q];
n) a VH region comprising an amino acid sequence having at least 90%, at least 95%, at least 97%, at least 99%, or 100% sequence identity to SEQ ID No: 19 and a VL region comprising an amino acid sequence having at least 90%, at least 95%, at least 97%, at least 99%, or 100% sequence identity to SEQ ID No: 20 [187];
o) a VH region comprising an amino acid sequence having at least 90%, at least 95%, at least 97%, at least 99%, or 100% sequence identity to SEQ ID No: 21 and a VL region comprising an amino acid sequence having at least 90%, at least 95%, at least 97%, at least 99%, or 100% sequence identity to SEQ ID No: 22 [608-01];
p) a VH region comprising an amino acid sequence having at least 90%, at least 95%, at least 97%, at least 99%, or 100% sequence identity to SEQ ID No: 23 and a VL region comprising an amino acid sequence having at least 90%, at least 95%, at least 97%, at least 99%, or 100% sequence identity to SEQ ID No: 24 [610-01];
q) a VH region comprising an amino acid sequence having at least 90%, at least 95%, at least 97%, at least 99%, or 100% sequence identity to SEQ ID No: 27 and a VL region comprising an amino acid sequence having at least 90%, at least 95%, at least 97%, at least 99%, or 100% sequence identity to SEQ ID No: 28 [613-08];
r) a VH region comprising an amino acid sequence having at least 90%, at least 95%, at least 97%, at least 99%, or 100% sequence identity to SEQ ID No: 29 and a VL region comprising an amino acid sequence having at least 90%, at least 95%, at least 97%, at least 99%, or 100% sequence identity to SEQ ID No: 30 [620-06]; and
s) a VH region comprising an amino acid sequence having at least 90%, at least 95%, at least 97%, at least 99%, or 100% sequence identity to SEQ ID No: 32 and a VL region comprising an amino acid sequence having at least 90%, at least 95%, at least 97%, at least 99%, or 100% sequence identity to SEQ ID No: 33 [726-M101L].

The conjugate for use according to the invention may comprise an antibody wherein said at least one binding region comprises a VH region and a VL region selected from the group consisting of;
a) a VH region comprising SEQ ID No: 1 and a VL region comprising SEQ ID No: 2 [107];
b) a VH region comprising SEQ ID No: 5 and a VL region comprising SEQ ID No: 6 [148];
c) a VH region comprising SEQ ID No: 34 and a VL region comprising SEQ ID No: 35 [733]
d) a VH region comprising SEQ ID No: 7 and a VL region comprising SEQ ID No: 9 [154];
e) a VH region comprising SEQ ID No: 10 and a VL region comprising SEQ ID No: 11 [171];
f) a VH region comprising SEQ ID No: 16 and a VL region comprising SEQ ID No: 18 [183];
g) a VH region comprising SEQ ID No: 25 and a VL region comprising SEQ ID No: 26 [613];
h) a VH region comprising SEQ ID No: 31 and a VL region comprising SEQ ID No: 33 [726];
i) a VH region comprising SEQ ID No: 3 and a VL region comprising SEQ ID No: 4 [140];
j) a VH region comprising SEQ ID No: 8 and a VL region comprising SEQ ID No: 9 [154-M103L];
k) a VH region comprising SEQ ID No: 12 and a VL region comprising SEQ ID No: 13 [172];
l) a VH region comprising SEQ ID No: 14 and a VL region comprising SEQ ID No: 15 [181];
m) a VH region comprising SEQ ID No: 17 and a VL region comprising SEQ ID No: 18 [183-N52Q];
n) a VH region comprising SEQ ID No: 19 and a VL region comprising SEQ ID No: 20 [187];
o) a VH region comprising SEQ ID No: 21 and a VL region comprising SEQ ID No: 22 [608-01];
p) a VH region comprising SEQ ID No: 23 and a VL region comprising SEQ ID No: 24 [610-01];
q) a VH region comprising SEQ ID No: 27 and a VL region comprising SEQ ID No: 28 [613-08];
r) a VH region comprising SEQ ID No: 29 and a VL region comprising SEQ ID No: 30 [620-06]; and
s) a VH region comprising SEQ ID No: 32 and a VL region comprising SEQ ID No: 33 [726-M101L].

The conjugate for use according to the invention may comprise an antibody, wherein said at least one binding region comprises a VH region and a VL region selected from the group consisting of:
a) a VH region comprising an amino acid sequence having at least 90%, at least 95%, at least 97%, at least 99%, or 100% sequence identity to SEQ ID No: 1 and a VL region comprising an amino acid sequence having at least 90%, at least 95%, at least 97%, at least 99%, or 100% sequence identity to SEQ ID No: 2 [107];
b) a VH region comprising an amino acid sequence having at least 90%, at least 95%, at least 97%, at least 99%, or 100% sequence identity to SEQ ID No: 25 and a VL region comprising an amino acid sequence having at least 90%, at least 95%, at least 97%, at least 99%, or 100% sequence identity to SEQ ID No: 26 [613];
c) a VH region comprising an amino acid sequence having at least 90%, at least 95%, at least 97%, at least 99%, or 100% sequence identity to SEQ ID No: 21 and a VL region comprising an amino acid sequence having at least 90%, at least 95%, at least 97%, at least 99%, or 100% sequence identity to SEQ ID No: 22 [608-01];
d) a VH region comprising an amino acid sequence having at least 90%, at least 95%, at least 97%, at least 99%, or 100% sequence identity to SEQ ID No: 23 and a VL region comprising an amino acid sequence having at least 90%, at least 95%, at least 97%, at least 99%, or 100% sequence identity to SEQ ID No: 24 [610-01];
e) a VH region comprising an amino acid sequence having at least 90%, at least 95%, at least 97%, at least 99%, or 100% sequence identity to SEQ ID No: 27 and a VL region comprising an amino acid sequence having at least 90%, at least 95%, at least 97%, at least 99%, or 100% sequence identity to SEQ ID No: 28 [613-08]; and
f) a VH region comprising an amino acid sequence having at least 90%, at least 95%, at least 97%, at least 99%, or 100% sequence identity to SEQ ID No: 29 and a VL region comprising an amino acid sequence having at least 90%, at least 95%, at least 97%, at least 99%, or 100% sequence identity to SEQ ID No: 30 [620-06].

In particular, the conjugate for use according to the invention may comprise an antibody, wherein said at least one binding region comprises a VH region and a VL region selected from the group consisting of:
a) a VH region comprising SEQ ID No: 1 and a VL region comprising SEQ ID No: 2 [107];
b) a VH region comprising SEQ ID No: 25 and a VL region comprising SEQ ID No: 26 [613];
c) a VH region comprising SEQ ID No: 21 and a VL region comprising SEQ ID No: 22 [608-01];
d) a VH region comprising SEQ ID No: 23 and a VL region comprising SEQ ID No: 24 [610-01];
e) a VH region comprising SEQ ID No: 27 and a VL region comprising SEQ ID No: 28 [613-08]; and
f) a VH region comprising SEQ ID No: 29 and a VL region comprising SEQ ID No: 30 [620-06].

The conjugate for use according to the invention may particularly comprise an antibody, wherein said at least one binding region comprises a VH region and a VL region selected from the group consisting of:
a) a VH region comprising an amino acid sequence having at least 90%, at least 95%, at least 97%, at least 99%, or 100% sequence identity to SEQ ID No: 5 and a VL region comprising an amino acid sequence having at least 90%, at least 95%, at least 97%, at least 99%, or 100% sequence identity to SEQ ID No: 6 [148];
b) a VH region comprising an amino acid sequence having at least 90%, at least 95%, at least 97%, at least 99%, or 100% sequence identity to SEQ ID No: 10 and a VL region comprising an amino acid sequence having at least 90%, at least 95%, at least 97%, at least 99%, or 100% sequence identity to SEQ ID No: 11 [171].

The conjugate for use according to the invention may comprise an antibody, wherein said at least one binding region comprises a VH region and a VL region selected from the group consisting of:
a) a VH region comprising SEQ ID No: 5 and a VL region comprising SEQ ID No: 6 [148];
b) a VH region comprising SEQ ID No: 10 and a VL region comprising SEQ ID No: 11 [171].

The conjugate for use according to the invention may comprise an antibody, wherein said at least one binding region comprises a VH region and a VL region selected from the group consisting of:
a) a VH region comprising an amino acid sequence having at least 90%, at least 95%, at least 97%, at least 99%, or 100% sequence identity to SEQ ID No: 7 and a VL region comprising an amino acid sequence having at least 90%, at least 95%, at least 97%, at least 99%, or 100% sequence identity to SEQ ID No: 9 [154];
b) a VH region comprising an amino acid sequence having at least 90%, at least 95%, at least 97%, at least 99%, or 100% sequence identity to SEQ ID No: 16 and a VL region comprising an amino acid sequence having at least 90%, at least 95%, at least 97%, at least 99%, or 100% sequence identity to SEQ ID No: 18 [183];
c) a VH region comprising an amino acid sequence having at least 90%, at least 95%, at least 97%, at least 99%, or 100% sequence identity to SEQ ID No: 34 and a VL region comprising an amino acid sequence having at least 90%, at least 95%, at least 97%, at least 99%, or 100% sequence identity to SEQ ID No: 35 [733].

The conjugate for use according to the invention may comprise an antibody, wherein said at least one binding region comprises a VH region and a VL region selected from the group consisting of:
a) a VH region comprising SEQ ID No: 7 and a VL region comprising SEQ ID No: 9 [154];
b) a VH region comprising SEQ ID No: 16 and a VL region comprising SEQ ID No: 18 [183];
c) a VH region comprising SEQ ID No: 34 and a VL region comprising SEQ ID No: 35 [733]

The conjugate for use according to the invention may in particular comprise an antibody, wherein said at least one binding region comprises a VH region comprising SEQ ID No: 31 and a VL region comprising SEQ ID No: 33 [726].

It is currently preferred that the conjugate for use according to the invention comprises an antibody, wherein said at least one binding region comprises a VH region comprising SEQ ID No: 1 and a VL region comprising SEQ ID No: 2 [107].

The antibody disclosed above may comprises a heavy chain, or two heavy chains, of an isotype selected from the group consisting of IgG1, IgG2, IgG3, and IgG4. Preferably, the antibody comprises a heavy chain, or two heavy chains, of the IgG1 isotype.

Preferably, the conjugate for use according to the invention, wherein the isotype is IgG1, optionally allotype IgG1m(f), the sequence of which is set forth in SEQ ID NO: 152.

Preferably, the conjugate for use according to the invention comprises an antibody, which is a full-length monoclonal antibody, such as a full-length monoclonal IgG1,κ antibody. In particular, the conjugate may comprise an antibody wherein the heavy chain IgG1 isotype, such as allotype IgG1m(f), is combined with either a kappa (κ) (SEQ ID NO: 153) or lambda (λ)(SEQ ID NO: 154) light chain.

The conjugate for use according to the invention may comprise an antibody, wherein the antibody is a human antibody or a humanized antibody.

In particularly preferred embodiments the conjugate for use according to the invention comprises an antibody, which is enapotamab or a biosimilar thereof. Enapotamab is the International Nonproprietary Name (INN) proposed by the World Health Organization for: Immunoglobulin G1-kappa, anti-[Homo sapiens AXL (AXL receptor tyrosine kinase, tyrosine-protein kinase receptor UFO)], Homo sapiens monoclonal antibody; gamma1 heavy chain (1-445) [Homo sapiens VH (IGHV3-23*01 (95.9%)—(IGHD)—IGHJ3*02 (100%)) [8.8.9] (1-116)—Homo sapiens IGHG1*03, G1m3 nG1m1 (CH1 R120 (213) (117-214), hinge (215-229), CH2 (230-339), CH3 E12 (355), M14 (357) (340-444), CHS K>del (445)) (117-445)], (219-215′)-disulfide with kappa light chain (1′-215′) [Homo sapiens V-KAPPA (IGKV3-20*01 (100%)—IGKJ2*01 (100%)) [7.3.9] (1′-108′)—Homo sapiens IGKC*01, Km3 A45.1 (154), V101 (192) (109′-215′)]; dimer (225-225″:228-228″)-bisdisulfide (WHO Drug Information, Vol. 31, No. 4, 2017; pp. 665-668). Enapotamab corresponds to antibody IgG1-AXL-107 used in the examples herein.

The antibody is typically connects to the cytotoxic agent via a linker. For antibodies with favourable internalization properties it may be preferred to select a linker, which is designed to be cleaved intracellularly. Once internalized, the ADC may be delivered to lysosomes, where effective drug release takes advantage of the catabolic environment found with these organelles. Thus, specialized linkers have been designed to be cleaved only in a specific microenvironment found in or on the target tumor cell or in the tumor microenvironment. Examples include linkers that are cleaved by acidic conditions, reducing conditions, or specific proteases.

Stability of the antibody-linker-drug in circulation is important because this allows antibody-mediated delivery of the drug to specific target cells. In addition, the long circulating half-life of the ADC provides exposure for several days to weeks post injection. Compounds that are conjugated through non-cleavable linkers and protease-cleavable linkers are generally more stable in circulation than compounds conjugated through disulfide and 47haracter linkers.

In the conjugate for use according to the invention, the cytotoxic agent may be linked to the antibody with a cleavable linker, such as N-succinimydyl 4-(2-pyridyldithio)-pentanoate (SSP), maleimidocaproyl-valine-citrulline-p-aminobenzyloxycarbonyl (mc-vc-PAB) or AV-1 K-lock valine-citrulline.

Alternatively, the cytotoxic agent may be linked to said antibody with a non-cleavable linker, such as succinimidyl-4(N-maleimidomethyl)cyclohexane-1-carboxylate (MCC) or maleimidocaproyl (MC).

The cytotoxic agent may be selected from any agent that is detrimental to (e.g., kills) cells. Suitable cytotoxic agents for forming immunoconjugates of the present invention include taxol, tubulysins, duostatins, cytochalasin B, gramicidin D, ethidium bromide, emetine, mitomycin, etoposide, tenoposide, vincristine, vinblastine, 48haracteri, doxorubicin, daunorubicin, dihydroxy anthracin dione, maytansine or an analog or derivative thereof, mitoxantrone, mithramycin, actinomycin D, 1 de¬hydro-testosterone, glucocorticoids, procaine, tetracaine, lidocaine, propranolol, and puromycin; calicheamicin or analogs or derivatives thereof; antimetabolites (such as methotrexate, 6 mercaptopurine, 6 thioguanine, cytarabine, 48haracteriz, 5 fluorouracil, 48haracteriz, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin; as well as duocarmycin A, duocarmycin SA, CC-1065 (a.k.a. rachelmycin), or analogs or derivatives of CC-1065), dolastatin, auristatin, pyrrolo[2,1-c][1,4] benzodiazepins (PDBs), indolinobenzodiazepine (IGNs) or analogues thereof, antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), anti-mitotic agents (e.g., tubulin-targeting agents), such as diphtheria toxin and related molecules (such as diphtheria A chain and active fragments thereof and hybrid molecules); ricin toxin (such as ricin A or a deglycosylated ricin A chain toxin), cholera toxin, a Shiga-like toxin (SLT I, SLT II, SLT IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca 48haracter proteins (PAPI, PAPII, and PAP S), 48haracter charantia inhibitor, curcin, crotin, Sapaonaria officinalis inhibitor, gelonin, mitogellin, restrictocin, phenomycin, and 48haracte toxins. Other suitable conjugated molecules include antimicrobial/lytic peptides such as CLIP, Magainin 2, mellitin, Cecropin, and P18; ribonuclease (Rnase), Dnase I, Staphylococcal enterotoxin A, pokeweed antiviral protein, diphtherin toxin, and Pseudomonas endotoxin.

In particular, the cytotoxic agent may be selected from the groupconsisting of DNA-targeting agents, e.g. DNA alkylators and cross-linkers, such as calicheamicin, duocarmycin, rachelmycin (CC-1065), pyrrolo[2,1-c][1,4] benzodiazepines (PBDs), and indolinobenzodiazepine (IGN); microtubule-targeting agents, such as duostatin, such as duostatin-3, auristatin, such as monomethylauristatin E (MMAE) and monomethylauristatin F (MMAF), dolastatin, maytansine, N(2′)-deacetyl-N(2′)-(3-marcapto-1-oxopropyl)-maytansine (DM1), and tubulysin; and nucleoside analogs; or an analogs, derivatives, or prodrugs thereof.

The cytotoxic agent may in particular be chosen from the group of microtubule targeting agents, such as auristatins and maytansinoids. Which inhibits mitosis (cell division). Microtubule-targeting agents disrupt or stabilize microtubules, which prevents formation of the mitotic spindle, resulting in mitotic arrest and apoptosis. The microtubule-targeting agents can be derived from e.g. natural substances such as plant alkaloids, and prevent cells from undergoing mitosis by disrupting or stabilizing microtubule polymerization, thus preventing formation of the mitotic spindle and subsequent cell division, resulting in inhibition of cancerous growth. Other examples of microtubule-targeting agents are paclitaxel, docetaxel, vinblastine, vincristine, vinorelbine, duostatins, tubulysins, and dolastatin.

It is preferred that the conjugate for use according to the invention has bystander kill capacity. In particular, the immunoconjugate may comprise a combination of a cytotoxic agent and a cleavable linker having bystander kill capacity, or a cytotoxic agent and a non-cleavable linker having bystander kill capacity.

The cytotoxic agent may in particular be MMAE or a functional analog or derivative thereof. Examples of analogs or derivatives of MMAE, which are useful in the context of the present invention include Duostatin-3 (Concortis/Levena), SYNstatinE (Synaffix), AGD-0182 (Astellas), Amberstatin-269 (Ambrx), Auristatin W (Bayer) and PF-06380101 (Pfizer).

In a particular embodiment, the cytotoxic agent is monomethyl auristatin E (MMAE);

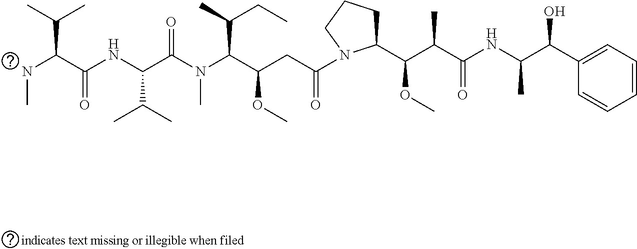 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

wherein the antibody is linked to MMAE at the nitrogen (N) on the left-hand side of the chemical structure above by the appropriate linker.

In preferred conjugates for use according to the invention, the linker is mc-vc-PAB and the cytotoxic agent is MMAE.

In one embodiment, the conjugate for use according to the invention comprises MMAE is linked to the antibody via a mc-vc-PAB linker, the cytotoxic agent and the linker having the chemical structure;

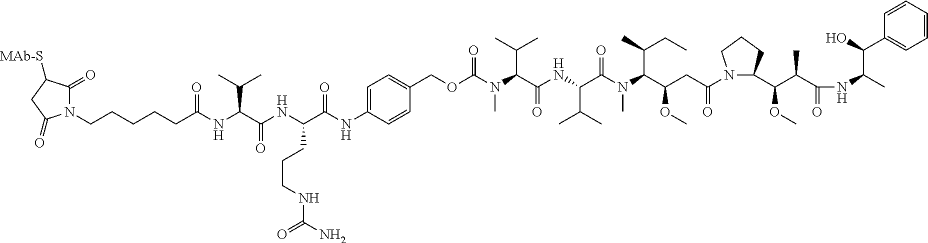

wherein Mab is the antibody.

The MMAE may in particular be conjugated to the antibody via a cysteine thiol or a lysine.

Alternatively, the cytotoxic agent monomethyl auristatin F (MMAF) may be linked to the antibody via a maleimidocaproyl (mc)-linker, wherein the combination of the cytotoxic agent and linker has the chemical structure;

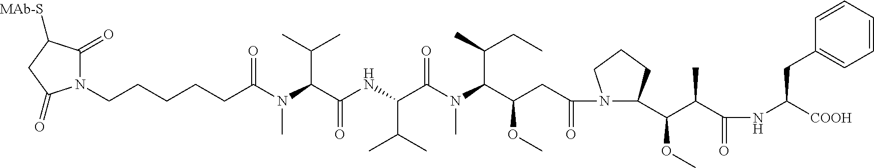

wherein Mab is the antibody.

As another alternative the cytotoxic agent may be duostatin3.

In certain embodiments, the conjugate for use according to any one of claims x-xx, said conjugate having a Drug-to-Antibody Ratio (DAR) which is within the range of 1-8, such as 1-7, 1-6, 1-5, 1-4, 1-3, 1-2, 2-7, 2-6, 2-5, 2-4, 2-3, 3-8, 3-7, 3-6, 3-5, 3-4, 4-8, 4-7, 4-6, 4-5, 5-8, 5-7, 5-6, 6-8, 6-7, or 7-8, the DAR being the average number of cytotoxic agent molecules conjugated to each antibody molecule. In currently preferred embodiments, the DAR is 3-5, such as 4.

The conjugate for use according to the invention may in particular be enapotamab vedotin or a biosimilar thereof. Enapotamab vedotin is the International Nonproprietary Name (INN) proposed by the World Health Organization for: immunoglobulin G1-kappa, anti-[Homo sapiens AXL (AXL receptor tyrosine kinase, tyrosine-protein kinase receptor UFO)], Homo sapiens monoclonal antibody conjugated to auristatin E; gamma1 heavy chain (1-445) [Homo sapiens VH (IGHV3-23*01 (95.9%)—(IGHD)—IGHJ3*02 (100%)) [8.8.9] 1-116)—Homo sapiens IGHG1*03, G1m3 nG1m1 (CH1 R120 (213) (117-214), hinge (215-229), CH2 (230-339), CH3 E12 (355), M14 (357) (340-444), CHS K>del (445)) (117-445)], (219-215′)-disulfide with kappa light chain (1′-215′) [Homo sapiens V-KAPPA (IGKV3-20*01 (100%)—IGKJ2*01 (100%)) [7.3.9] (1′-108′)—Homo sapiens IGKC*01, Km3 A45.1 (154), V101 (192) (109′-2151]; dimer (225-225″:228-228″)-bisdisulfide; conjugated, on an average of 4 cysteinyl, to monomethylauristatin E (MMAE), via a cleavable maleimidocaproyl-valyl-citrullinyl-paminobenzyloxycarbonyl (mc-val-cit-PABC) type linker. (WHO Drug Information, Vol. 31, No. 4, 2017; pp. 665-668). Enapotamab vedotin corresponds to IgG1-AXL-107-vcMMAE, which is used in the examples herein.

In conjugates for use according to the invention the antibody may alternatively comprise at least one binding region comprising a variable heavy chain (VH) region and a variable light chain (VL) region selected from the group consisting of:
a) a VH region comprising an amino acid sequence having at least 90%, at least 95%, at least 97%, at least 99%, or 100% sequence identity to SEQ ID No: 139 and a VL region comprising an amino acid sequence having at least 90%, at least 95%, at least 97%, at least 99%, or 100% sequence identity to SEQ ID No: 140;
b) a VH region comprising an amino acid sequence having at least 90%, at least 95%, at least 97%, at least 99%, or 100% sequence identity to SEQ ID No: 141 and a VL region comprising an amino acid sequence having at least 90%, at least 95%, at least 97%, at least 99%, or 100% sequence identity to SEQ ID No: 142;
c) a VH region comprising an amino acid sequence having at least 90%, at least 95%, at least 97%, at least 99%, or 100% sequence identity to SEQ ID No: 141 and a VL region comprising an amino acid sequence having at least 90%, at least 95%, at least 97%, at least 99%, or 100% sequence identity to SEQ ID No: 143;
d) a VH region comprising an amino acid sequence having at least 90%, at least 95%, at least 97%, at least 99%, or 100% sequence identity to SEQ ID No: 144 and a VL region comprising an amino acid sequence having at least 90%, at least 95%, at least 97%, at least 99%, or 100% sequence identity to SEQ ID No: 142; and
e) a VH region comprising an amino acid sequence having at least 90%, at least 95%, at least 97%, at least 99%, or 100% sequence identity to SEQ ID No: 144 and a VL region comprising an amino acid sequence having at least 90%, at least 95%, at least 97%, at least 99%, or 100% sequence identity to SEQ ID No: 143.

Further disclosure of such antibodies are provided in WO2016166296. These antibodies may in particular be conjugated to a pyrrolobenzodiazepine, such as a pyrrolobenzodiazepine (PBD) dimer.

In further embodiments, the conjugate for use according to the invention is ADCT-601 (ADC Therapeutics).

Alternatively, the conjugate for use according to the invention is a conjugate of an antibody comprising at least one binding region comprising a variable heavy chain (VH) region and a variable light chain (VL) region, the VH region and the VL region comprising amino acids sequences encoded by the nucleic acid sequences set forth in:
a) SEQ ID Nos: 145 and 146, respectively;
b) SEQ ID Nos: 145 and 147, respectively;
c) SEQ ID Nos: 145 and 148, respectively;
d) SEQ ID Nos: 145 and 149, respectively;
e) SEQ ID Nos: 150 and 146, respectively;
f) SEQ ID Nos: 150 and 147, respectively;
g) SEQ ID Nos: 150 and 148, respectively;
h) SEQ ID Nos: 150 and 149, respectively;
i) SEQ ID Nos: 151 and 146, respectively;
j) SEQ ID Nos: 151 and 147, respectively;
k) SEQ ID Nos: 151 and 148, respectively; and
l) SEQ ID Nos: 151 and 149, respectively.

Additional disclosure of such antibodies can be found in WO2017180842. Each of these antibodies may in particular be conjugated to a microtubule targeting agent, such as an auristatin (e.g. MMAE) or auristatin peptide analog or derivate, or a maytansinoid.

The conjugate for use according to the invention may in particular be CAB-AXL-ADC/BA3011 (BioAtla). CAB-AXL-ADC/BA3011 is an anti-AXL humanized monoclonal antibody conjugated to monomethyl auristatin E using a cleavable linker. It specifically binds to AXL under conditions found within the microenvironment of a tumor. Further disclosure of this conjugate is provided by J R Ahnert et al., DOI: 10.1200/JCO.2018.36.15_suppl.TPS12126 Journal of Clinical Oncology 36, no. 15_suppl (2018).

The conjugate for use according to any one of the preceding claims, wherein the inhibitor of PD-1 and/or PD-L1 is an inhibitor of the interaction between PD-1 and its ligand; e.g. PD-L1.

The conjugate for use according to the invention may be combined with an inhibitor of PD-1 and/or PD-L1, which is an antibody or comprises an antibody or antigen-binding fragment thereof. The antibody may in particular be an antagonistic antibody or antigen-binding fragment (i.e. an antibody or antigen-binding fragment, which reduces or abolishes ligand binding and/or activation of PD-1 and/or signaling through the PD-1 pathway.

The inhibitor of PD-1 and/or PD-L1 may be selected from the group consisting of pembrolizumab (Merck & Co), CBT-501 (genolimzumab; Genor Bio/CBT Pharma), nivolumab (BMS), REGN2810 (Cemiplimab; Regeneron), BGB-A317 (Tislelizumab; BeiGene/Celgene), Amp-514 (MED10680) (Amplimmune), TSR-042 (Dostarlimab; Tesaro/AnaptysBio), JNJ-63723283/JNJ-3283 (Johnson & Johnson), PF-06801591 (Pfizer), JS-001 (Tripolibamab/Toripalimab; Shanghai Junshi Bio), SHR-1210/INCSHR-1210 (Camrelizumab; Incyte corp), PDR001 (Spartalizumab; Novartis), BCD-100 (BioCad), AGEN2034 (Agenus), IBI-308 (Sintilimab; Innovent Biologics), B1-754091 (Boehringer Ingelheim), GLS-010 (WuXi/Arcus), LZM-009 (Livzon MabPharm), AMG-404 (Amgen), CX-188 (CytomX), ABBV-181 (Abbvie), BAT-1306 (BioThera), JTX-4014 (Jounce Therapeutics), AK-103 (Akeso Bio), AK-105 (Akeso Bio), MGA-012 (Macrogenics/Incyte), Sym-021 (Symphogen), AB122 (Arcus Biosciences/Strata Oncology), CS1003 (C-Stone), 609A (Sunshine Guojian), hAB21 (Suzhou Stainwei Biotech), SCT-110A (Sinocelltech), HLX-10 (Shanghai Henlius Biotech), HX-008 (Taizhou Hanzhong Biomedical).

The inhibitor of PD-1 and/or PD-L1 may in particular be selected from the group consisting of pembrolizumab, genolimzumab, nivolumab, cemiplimab, Tislelizumab,

The conjugate for use according to any one of the preceding claims, wherein the inhibitor of PD-1 and/or PD-L1 is pembrolizumab (Merck & Co).

The inhibitor of PD-1 and/or PD-L1 may in particular comprise an antibody, or antigen-binding fragment thereof, capable of binding to PD-L1.

The inhibitor of PD-1 and/or PD-L1 may be selected from the group consisting of RG7446/MPDL-3280A (atezolizumab; Roche), MSB-0010718C (avelumab; Merck Serono/Pfizer) and MEDI-4736 (durvalumab; AstraZeneca), KN-035 (envafolimab; 3Dmed/Alphamab Co.), CX-072 (CytomX), LY-3300054 (Eli Lilly), STI-A1014 (Sorrento/Lees Pharm), A167 (Harbour BioMed/Kelun biotech), BGB-A333 (BeiGene), MSB0011359C (M-7824) (Bintrafusp alfa; Merck KgaA), FAZ053 (Novartis), BCD-135 (Biocad), HLX-20 (Shanghai Henlius Bio), AK-106 (Akeso), KL-A167 (Kelun), SHR-1316 (Atridia), CA-170 (Aurigene/Curis), LP-002 (Lepu Pharmaceuticals), MSB2311 (MabSpace), CK-301 (Cosibelimab; Checkpoint Therapeutics and TG Ther.), CS1001/WBP-3155 (C-Stone Pharmaceuticals), IMC-001 (ImmuneOncia/Sorrento), WBP3155 (C-Stone Pharm), ZKAB001 (Sorrento/Lee's Pharma), JS-003 (Shanghai Junshi Biosciences), CBT-502 (CBT Pharmaceuticals), GS-4224 (Gilead), TG-1501 (TG Therapeutics), CBT-502 (CBT Pharmaceuticals).

The inhibitor of PD-1 and/or PD-L1 may in particular comprise atezolizumab, avelumab, durvalumab and envafolimab.

The conjugate for use according to the invention may in particular be a conjugate that has antitumor activity or is able to induce tumor regression in Non-Small Cell Lung Cancer (NSCLC) and/or melanoma xenograft models, such as a BLM melanoma xenograft model or a LCLC-103H xenograft model.

In the BLM melanoma xenograft model, the conjugate may be injected intravenously as a single dose of 4 mg/kg, together with 5×10⁶ CD8 T-cells, such as CD8 T-cells specific for Melanoma-associated antigen recognized by T cells (MART-1).

In the LCLC-103H xenograft model the conjugate may be injected intravenously as a single dose of 1 mg/kg, together with 5×10⁶ CD8 T-cells, such as CD8 T-cells specific for Melanoma-associated antigen recognized by T cells (MART-1).

The NSCLC and/or melanoma xenograft model may be a model, which is resistant to treatment with one or more inhibitors of PD-1 and/or PD-L1, such as one ore more inhibitors of PD-1 and/or PD-L1 as defined above.

The conjugate for use according to the invention may be a conjugate that has antitumor activity in a BLM melanoma xenograft model, wherein the BLM melanoma xenograft model is generated as described in Example 3 herein or is generated essentially as described in Example 3 herein.

The conjugate for use according to the invention may be a conjugate that has antitumor activity in a NSCLC xenograft model, wherein the NSCLC xenograft model is generated as described in Example 4 herein or is generated essentially as described in Example 4 herein.

The immunogenic cell death and/or the tumor-associated inflammation may be 54haracterized by
a) increased release of ATP from cells of said tumor,
b) secretion of high-mobility group box 1 (HMGB1) from cells of said tumor, and/or
c) cell surface expression of Calreticulin on cells of said tumor.

The ability of said antibody to induce immunogenic cell death and/or tumor-associated inflammation may be determined by measuring
a) release of ATP,
b) secretion of high-mobility group box 1 (HMGB1), and/or
c) cell surface expression of Calreticulin,

in a cancer cell line. As shown in the examples herein the cancer cell line may be a human Non-Small Cell Lung Cancer cell line or a human breast cancer cell line.

The release of ATP, secretion of HGMB1 and/or cell surface expression of Calreticulin may be determined as described in Example 7 herein.

The human Non-Small Cell Lung Cancer cell line may be LCLC-103H and/or the human breast cancer cell line may be MDA-MB-231.

The release of ATP may determined be in a process comprising
i) Establishing a culture of said cancer cell line in 24 well plates and culturing the cells to for 3-4 hours at 37° C.,
ii) Adding said conjugate to the cells in an amount corresponding to 2 μg/mL, and incubating the cells for 48 hours at 37° C.,
iii) Washing the cells in phosphate buffered saline (PBS)
iv) Incubating the cells in 100% PBS or 70% PBS,
v) Harvesting the cells and separating cells and supernatant by centrifugation at 1000 rpm for 2 minutes at room temperature, and
vi) Measuring ATP in the supernatant

The secretion of HMGB1 may be determined in a process comprising:
i) Establishing a culture said cancer cell line in 24 well plates and culturing the cells to for 3-4 hours at 37° C., Adding said conjugate to the cells in an amount corresponding to 2 μg/mL and incubating the cells for 48 hours at 37° C.,
iii) Separating cells and supernatant from the culture by centrifugation at 1000 rpm for 3 minutes at room temperature, and
iv) Measuring HMGB1 in the supernatant, such as by enzyme-linked immunosorbent assay (ELISA).

The cell surface expression of Calreticulin may be determined by a process comprising:
i) Establishing a culture of said cancer cell line, grown to 70-80% confluency and incubating the cancer cell line with the conjugate in amounts corresponding of 2 μg/ml for 48 hours at 37° C., Collecting cells from the culture and washing the cells in FACS buffer (phosphate buffered saline (PBS)/0.1 w/w bovine serum albumin (BSA)/0.02% w/w azide),
iii) Incubating the cells with phycoerythrin (PE)-conjugated mouse anti-human calreticulin antibody for 30 minutes at 4° C. in darkness,
iv) Washing the cells in FACS buffer and analyzing the cells by flow cytometry.

The subject to be treated according to the present invention is preferably a human subject.

The actual dosage levels of the conjugate and the inhibitor of PD-1 and/or PD-L1 when used according to the invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular antibody capable of binding Axl, the cytotoxic agent and the inhibitor of PD-1 and/or PD-L1b. It will further depend on the route of administration, the time of administration, the rate of excretion of the particular compounds being employed, the duration of the treatment, the age, sex, weight, condition, general health and prior medical history of the subject being treated.

The conjugate of the antibody capable of binding to Axl and cytotoxic agent may be administered to said subject in therapeutically effective amounts and frequencies; such as
In at least one cycle comprising administration of the conjugate once every three weeks, such as on day 1 of a cycle of 21 days; or
in at least one cycle comprising administration of the conjugate once a week for three consecutive weeks followed by a one-week resting period without any administration of ADC so that each cycle time is 28 days including the resting period, such as on days 1, 8 and 15 in the cycle of 28 days.

The the dose of the conjugate in said cycle of 21 days may be between 0.6 mg/kg and 4.0 mg/kg of the subject's body weight, such as between 0.6 mg/kg and 3.2 mg/kg of the subject's body weight, such as at a dose of about 0.6 mg/kg or at a dose of about 0.8 mg/kg or at a dose of about 1.0 mg/kg or at a dose of about 1.2 mg/kg or at a dose of about 1.4 mg/kg or at a dose of about 1.6 mg/kg or at a dose of about 1.8 mg/kg or at a dose of about 2.0 mg/kg or at a dose of about 2.2 mg/kg or at a dose of about 2.4 mg/kg or at a dose of about 2.6 mg/kg or at a dose of about 2.8 mg/kg or at a dose of about 3.0 mg/kg or at a dose of about 3.2 mg/kg.

The dose of the conjugate in said cycle of 28 days may be between 0.45 mg/kg and 2.0 mg/kg of the subject's body weight, such as at a dose of 0.45 mg/kg or at a dose of 0.5 mg/kg or at a dose of 0.6 mg/kg or at a dose of 0.7 mg/kg or at a dose of 0.8 mg/kg or at a dose of 0.9 mg/kg or at a dose of 1.0 mg/kg or at a dose of 1.1 mg/kg or at a dose of 1.2 mg/kg or at a dose of 1.3 mg/kg or at a dose of 1.4 mg/kg or at a dose of 1.5 mg/kg or at a dose of 1.6 mg/kg or at a dose of 1.7 mg/kg or at a dose of 1.8 mg/kg or at a dose of 1.9 mg/kg or at a dose of 2.0 mg/kg.

The number of cycles of 21 days or the number of cycles of 28 days may be between 2 and 48, such as between 2 and 36, such as between 2 and 24, such as between 2 and 15, such as between 2 and 12, such as 2 cycles, 3 cycles, 4 cycles, 5 cycles, 6 cycles, 7 cycles, 8 cycles, 9 cycles, 10 cycles, 11 cycles or 12 cycles.

The conjugate may be administered for at least four treatment cycles of 28 days, wherein the antibody or ADC in each treatment cycle is administered once a week at a dose of 0.45 mg/kg body weight, such as at a dose of 0.6 mg/kg body weight, 0.8 mg/kg body weight, 1.0 mg/kg body weight, 1.2 mg/kg body weight, 1.4 mg/kg body weight, 1.6 mg/kg body weight, 1.8 mg/kg body weight, or such as 2.0 mg/kg body weight for three consecutive weeks followed by a resting week without any administration of the antibody or ADC.

In particular, the conjugate may be administered to the subject at a dose of about 2.0-about 2.4 mg/kg body weight once every three weeks or by weekly dosing of about 0.6-about 1.4 mg/kg body weight for three weeks, optionally followed by one treatment-free week.

In currently preferred embodiments, the conjugate for use according to the invention may be administered to the subject at a dose of about 2.2 mg/kg body weight once every three weeks or by weekly dosing of about 1.0 mg/kg body weight for three weeks, optionally followed by one treatment-free week.

The treatment according to the invention may be continued at least until said subject has experienced progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the first dose of the conjugate.

Alternatively, the treatment may be continued until disease progression.

The conjugate may be administered to the subject at a dose of about 1.8-about 2.6 mg/kg body weight once every three weeks or by weekly dosing of about 0.8-about 1.2 mg/kg body weight for three weeks, optionally followed by one treatment-free week.

When used according to the present invention the conjugate of a cytotoxic agent and an antibody or antigen-binding fragment thereof and the inhibitor of PD-1 and/or PD-L1 may be administered to the subject by the same route of administration or by different routes of administration. Hence, the conjugate of a cytotoxic agent and antibody or antigen-binding fragment thereof and/or the inhibitor of PD-1 and/or PD-L1 may be administered parenterally; i.e. by a mode of administration other than enteral and topical administration; usually by injection, and include epidermal, intravenous, intramuscular, intra-arterial, intrathecal, intracapsular, intra-orbital, intracardiac, intradermal, intraperitoneal, intratendinous, transtracheal, subcutaneous, subcuticular, intra-articular, subcapsular, subarachnoid, intraspinal, intracranial, intrathoracic, epidural and intrasternal injection and infusion. In particular, the the conjugate of a cytotoxic agent and an antibody or antigen-binding fragment thereof and the inhibitor of PD-1 and/or PD-L1 may be administered by intravenous or subcutaneous injection or infusion.

The conjugate of a cytotoxic agent and an antibody or antigen-binding fragment thereof and the inhibitor of PD-1 and/or PD-L1 may are preferably administered by intravenous injection or infusion. Typically, intravenous infusion of each dose would take place over a time period of 30 minutes or 60 minutes.

For currently approved inhibitors of PD-1 or PD-L1 the skilled person will be aware of the dosage regime according to which the particular inhibitor should be administered. For instance, Avelumab may be dosed at 1200 mg as an intravenous infusion at a frequency of one dose every three weeks (Q3W). Nivolumab may be dosed at 480 mg at a frequency of one dose every four weeks (Q4W) or at 240 mg at a frequency of one dose every two weeks (Q2W).

The recommended dose of pembrolizumab 200 mg at a frequency of one dose every 3 weeks (Q3W) or 400 mg at a frequency of one dose every 6 weeks (Q6W). For use in combination therapy, the recommended dose is 200 mg at a frequency of one dose every 3 weeks.

The cancer to be treated according to the present invention may be a solid tumor, such as a metastasic, solid tumor, such as a metastasic, locally advanced tumor.

The cancer may in particular be selected from the group consisting of colorectal cancer, such as colorectal carcinoma and colorectal adenocarcinoma; bladder cancer, bone cancer such as chondrosarcoma; breast cancer such as triple-negative breast cancer; cancers of the central nervous system such as glioblastoma, astrocytoma, neuroblastoma; cervical cancer, connective tissue cancer, endometrium cancer, fibroblast cancer, gastric cancer such as gastric carcinoma; head and neck cancer, kidney cancer, liver cancer such as hepatocellular carcinoma; lung cancer such as NSCLC and lung squamous cell carcinoma; muscle cancer, neural tissue cancer, ovarian cancer, pancreatic cancer such as pancreatic ductal carcinoma and pancreatic adenocarcinoma; skin cancer such as malignant melanoma; soft tissue sarcoma and mesothelioma.

In some embodiments, the cancer is selected from the group consisting of non-small cell lung cancer (NSCLC), melanoma, Sarcoma, cervical cancer, endometrial cancer and ovarian cancer, pancreatic cancer, bladder cancer, and head and neck cancer.

In particular embodiments, the cancer is non-small cell lung cancer (NSCLC).

In some embodiments the tumor and/or cancer is characterized by expression of PD-L1 and/or PD-1. Expression of PD-L1 and PD-1 may be determined for instance in immunohistochemistry (IHC) assays using anti-PD-L1 or anti-PD-1 antibodies, such as anti-human PD-L1 or anti-human PD-1 antibodies.

Further, the tumor or cancer to be treated may be characterized by having a T cell infiltrate; i.e. as being a T cell-positive or inflamed tumor. Alternatively, the tumor or cancer may be characterized by not having a T cell infiltrate; i.e. as being a T cell-negative or non-inflamed tumor.

The combined treatment with the conjugate and the inhibitor of PD-1 and/or PD-L1 may in particular be offered to a subject, which has received prior treatment with a PD-1 pathway inhibitor; e.g. an inhibitor of PD-1 and/or PD-L1 as defined herein above.

The cancer may previously have been treated with a PD-1 pathway inhibitor, such as an inhibitor of PD-1 and/or PD-L1 as defined above.

The cancer and/or the subject may be resistant to, may have failed to respond to, or may have relapsed from prior treatment with a PD-1 pathway inhibitor, such as an inhibitor of PD-1 and/or PD-L1 as defined above.

In particular embodiments, treatment with a PD-1 pathway inhibitor, such as an inhibitor of PD-1 and/or PD-L1 as defined above, was the last treatment prior to treatment with said conjugate and inhibitor of PD-1 and/or PD-L1 as defined according to the invention.

In certain embodiments, the cancer and/or the subject has primary (de novo) or acquired resistance to a PD-1 pathway inhibitor, such as an inhibitor of PD-1 and/or PD-L1 as defined above.

The resistance to, failure to respond to and/or relapse from treatment with a PD-1 pathway inhibitor may be determined according to the Response Evaluation Criteria In Solid Tumors; version 1.1 (RECIST Criteria v1.1). The RECIST Criteria are set forth in the table below.

Definition of Response (RECIST Criteria v1.1)

| | Category | Criteria |
|---|---|---|
| Based on target | Complete | Disappearance of all target lesions. Any |
| lesions | Response | pathological lymph nodes must have |
| | (CR) | reduction in short axis to <10 mm. |
| | Partial | ≥30% decrease in the sum of the LD |
| | Response | of target lesions, taking as reference |
| | (PR) | the baseline sum LD. |
| | Stable | Neither sufficient shrinkage to qualify |
| | Disease | for PR nor sufficient increase to qualify |
| | (SD) | for PD, taking as reference the smallest |
| | | sum of LDs since the treatment started. |
| | Progressive | ≥20% increase in the sum of the LDs |
| | Disease | of target lesions, taking as reference |
| | (PD) | the smallest sum of the LDs recorded |
| | | since the treatment started or the |
| | | appearance of one or more new lesions. |
| Based on non- | CR | Disappearance of all non-target lesions |
| target lesions | | and normalization of tumor marker |
| | | level. All lymph nodes must be non- |
| | | pathological in size (<10 mm short axis). |
| | SD | Persistence of one or more non-target |
| | | lesion(s) or/and maintenance |
| | | of tumor marker level above |
| | | the normal limits. |
| | PD | Appearance of one or more new lesions |
| | | and/or unequivocal progression of |
| | | existing non-target lesions. |

When entering into treatment with the conjugate of an antibody capable of binding to Axl in combination with an inhibitor of PD-1 and/or PD-L1, the said subject may have Stable Disease (SD) or Progressive Disease (PD); such as determined according to the RECIST Criteria v1.1.

A further aspect of the invention provides an inhibitor of PD-1 and/or PD-L1 for use in treating cancer in a subject, in combination with a conjugate of a cytotoxic agent and an antibody or antigen-binding fragment thereof capable of binding to human Axl; e.g. human Axl having the sequence set forth in SEQ ID NO: 130.

The inhibitor of PD-1 and/or PD-L1 may be as defined above or may have any of the features or characteristics defined above for inhibitors of PD-1 and/or PD-L1.

The antibody may be as defined above or may have any of the features or characteristics defined above for antibodies capable of binding Axl.

The cytotoxic agent may be as defined above or may have any of the features or characteristics defined above for cytotoxic agents.

The cancer may be a cancer as defined above.

The subject to whom the treatment is offered may be a subject as defined above.

The PD-1 pathway inhibitor and/or the conjugate may be administered to the subject as defined above.

The invention also provides a method of potentiating the therapeutic efficacy or anti-tumor activity of PD-1 and/or PD-L1 inhibition, comprising administering to a subject in need thereof, such as a subject suffering from cancer and/or carrying a tumor, a conjugate of a cytotoxic agent and an antibody capable of binding to human Axl (e.g. human Axl having the sequence set forth in SEQ ID NO: 1), thereby inducing immunogenic cell death and/or tumor-associated inflammation; e.g. tumor-associated inflammation associated with immunogenic cell death.

The method may further be used for potentiating the clinical efficacy of anti PD-1 and/or anti PD-L1 therapy provided to said subject.

The conjugate may be administered in combination with an inhibitor of programmed cell death-1 (PD-1) and/or programmed death-ligand 1 (PD-L1).

A further aspect of the invention provides a method of treating cancer comprising administering to a subject in need thereof
an inhibitor of PD-1 and/or PD-L1; and
a conjugate of a cytotoxic agent and an antibody or antigen-binding fragment thereof capable of binding to human Axl; e.g. human Axl having the sequence set forth in SEQ ID NO: 130.

In the method according to the invention, the inhibitor of PD-1 and/or PD-L1 may be as defined above and/or may have any of the features or characteristics defined above in relation of inhibitors of PD-1 and/or PD-L1.

In the method according to the invention, the antibody capable of binding Axl may be as defined above defined and/or may have any of the features or characteristics defined above in relation to antibodies being capable of binding to Axl.

In the method according to the invention, the cytotoxic agent may be as defined above and/or may have any of the features or characteristics defined above in relation to cytotoxic agents.

In the method according to the invention, the cancer may be as defined above.

In the method according to the invention, the subject may be as defined above.

In the method according to the invention, the inhibitor of PD-1 and/or PD-L1 and/or the conjugate may be administered to the subject is as defined above.

Tumor-associated inflammation may be characterized as defined above and/or may be determined as set forth above.

A still further aspect of the invention provides a composition comprising a composition or formulation comprising a conjugate of an antibody or antigen-binding fragment thereof capable of binding to human Axl; e.g. human Axl having the sequence set forth in SEQ ID NO: 130, and an inhibitor of PD-1 and/or PD-L1. The composition may in particular be a pharmaceutical composition comprising the conjugate of an antibody or antigen-binding fragment thereof capable of binding to human Axl and the inhibitor of PD-1 and/or PD-L1, and a pharmaceutically acceptable carrier.

The composition or pharmaceutical composition may be formulated with the carrier, excipient and/or diluent as well as any other components suitable of pharmaceutical compositions, including known adjuvants, in accordance with conventional techniques such as those disclosed in Remington: The Science and Practice of Pharmacy, 19^{th} Edition, Gennaro, Ed., Mack Publishing Co., Easton, Pa., 1995. The pharmaceutically acceptable carriers or diluents as well as any known adjuvants and excipients should be suitable for the antibody or antibody conjugate of the present invention and the chosen mode of administration. Suitability for carriers and other components of pharmaceutical compositions is determined based on the lack of significant negative impact on the desired biological properties of the chosen compound or pharmaceutical composition of the present invention (e.g., less than a substantial impact [10% or less relative inhibition, 5% or less relative inhibition, etc.] upon antigen binding).

A composition, such as a pharmaceutical composition, of the present invention may include diluents, fillers, salts, buffers, detergents (e. g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

Pharmaceutically acceptable carriers include any and all suitable solvents, dispersion media, coatings, antibacterial and antifungal agents, isotonicity agents, antioxidants and absorption-delaying agents, and the like that are physiologically compatible with a compound of the present invention.

Examples of suitable aqueous and non-aqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

Compositions, such as pharmaceutical compositions, of the present invention may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Compositions, such as pharmaceutical compositions, of the present invention may also comprise isotonicity agents, such as sugars, polyalcohols, such as mannitol, sorbitol, glycerol or sodium chloride in the compositions.

The compositions, such as the pharmaceutical compositions, of the present invention may also contain one or more adjuvants appropriate for the chosen route of administration such as preservatives, wetting agents, emulsifying agents, dispersing agents, preservatives or buffers, which may enhance the shelf life or effectiveness of the composition. The combination of compounds of the present invention may be prepared with carriers that will protect the compound against rapid release, such as a controlled release formulation, including implants, transdermal patches, and micro-encapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, poly-ortho esters, and polylactic acid alone or with a wax, or other materials well known in the art. Methods for the preparation of such formulations are generally known to those skilled in the art, see e.g. Sustained and Controlled Release Drug Delivery Systems, J. R. Robinson, ed., Marcel Dekker, Inc., New York, 1978.

In one embodiment, the combination of compounds of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except in so far as any conventional media or agent is incompatible with the active compound, use thereof in the compositions of the present invention is contemplated. Other active or therapeutic compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compounds in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

The composition, such as the pharmaceutical composition, of the present invention may contain one or more antibody antibody-drug conjugates (ADCs), in combination with one or more inhibitors of PD-1 and/or PD-L1, optionally in combination with one or more additional therapeutic compound(s).

In the composition according to the invention, the one or more antibody antibody-drug conjugates (ADCs) may in particular comprise an antibody as defined or disclosed above.

In the composition according to the invention, the one or more inhibitors of PD-1 and/or PD-L1 may in particular be selected form the inhibitors of PD-1 and/or PD-L1 defined or disclosed above.

In the composition according to the invention, the cytotoxic agent may in particular be as disclosed or defined above.

The composition may be administered by any suitable route and mode. Suitable routes of administering a compound of the present invention in vivo and in vitro are well known in the art and may be selected by those of ordinary skill in the art.

In currently preferred embodiments, the composition has a pH of about 5 to about 7 and comprises
(a) from about 5 mg/mL to about 30 mg/mL of the conjugate and
(b) from about 10 mM to about 50 mM histidine;
(c) from about 15 mM to about 120 mM sucrose or trehalose; and
(d) from about 150 mM to about 180 mM mannitol or glycine.

In particular, the composition may have a pH of 6 and comprise:
10 mg/mL of the conjugate,
30 mM histidine,
88 mM sucrose; and
165 mM mannitol

A further aspect of the invention provides a kit of parts comprising a conjugate of an antibody or antigen-binding fragment thereof capable of binding to human Axl; e.g. human Axl having the sequence set forth in SEQ ID NO: 130, and a programmed cell death-1 (PD-1) pathway inhibitor. Instructions for use may further be included in the kit

In the kit according to the invention, the inhibitor of PD-1 and/or PD-L1 may have any of the features and characteristics defined above.

Further, in the kit according to the invention, the antibody capable of binding to Axl may be as defined above, and/or have any of the features and characteristics set forth above.

Finally, in the kit according to the invention, the cytotoxic agent may be as defined above, and/or have any of the features and characteristics set forth above.

The present invention is further illustrated by the following examples which should not be construed as further limiting.

### EXAMPLES

### Example 1—Antibody Production

AXL-specific antibodies as disclosed in WO 2016/005593, including IgG1-AXL-107 (Enapotamab)), and isotype control antibody IgG1-b12 (Barbas, C F. J Mol Biol. 1993 Apr. 5; 230(3):812-23) were expressed as IgG1,κ. Plasmid DNA mixtures encoding heavy and light chains of antibodies were transiently transfected to Expi293F cells (Life technologies, USA) using 293fectin (Life technologies) essentially as described by Vink et al. (Vink et al., Methods, 65 (1), 5-10 2014). Antibodies were purified by immobilized protein G chromatography. Protein batches were analyzed by a number of bioanalytical assays including SDS-PAGE, size exclusion chromatography and measurement of endotoxin levels. Purified antibodies were conjugated with maleimidocaproyl-valine-citrulline-p-aminobenzoyloxycarbonyl-monomethyl auristatin E (vcMMAE) containing a protease-cleavable valine-citrulline dipeptide as described (Doronina, S. O. et al. (2003) Nat. Biotechnol. 21, 778-784). The average drug-antibody ratio was 4:1. The anti-PD1 antibody pembrolizumab (KEYTRUDA®, MSD) was commercially obtained from SelleckChem (Cat. No.: A2005).

### Example 2—Isolation and Generation of Human, MART-1-Specific CD8 T Cells

MART-1 (1D3) T cell receptor (TCR) retrovirus was produced in a packaging cell line as described previously (Jorritsma et al. (2007) Blood; 110, 3564-3572). Peripheral blood mononuclear cells were isolated from healthy donor buffycoats (Sanquin, Amsterdam, the Netherlands) by density gradient centrifugation using Lymphoprep (Stem Cell Technologies). CD8+ T cells were purified using CD8 Dynabeads (Thermo Fisher Scientific), activated for 48 hours on a non-tissue culture treated 24-well plate that was pre-coated overnight with αCD3 and αCD28 antibodies (eBioscience, 16-0037-85 and 16-0289-85, respectively) at 2×10⁶ per well. Activated CD8 T cells were harvested and mixed with TCR retrovirus (MART-1 T cells) or mock retrovirus (control T cells) and spinfected on a Retronectin coated (Takara, 25 μg per well) non-tissue culture treated 24-well plate for 2 hours at 2000×g. After 24 hours, T cells were harvested and maintained in RPMI (Gibco) containing 10% human serum (One Lambda), 100 units per mL of penicillin, 100 μg per mL of streptomycin, 100 units per mL IL-2 (Proleukin, Novartis), 10 ng per mL IL-7 (ImmunoTools, Friesoythe, Germany) and 10 ng per mL IL-15 (ImmunoTools).

### Example 3—Anti-Tumor Activity of IgG1-AXL-107-vcMMAE in Combination with Anti-PD-1 in the BLM Melanoma Xenograft Model that is Resistant to Anti-PD-1 Treatment

The anti-tumor activity of IgG1-AXL-107-vcMMAE in combination with anti-PD1 (pembrolizumab) was evaluated in the BLM human melanoma cell line-derived xenograft model in mice, which systemically received human T-cells that were engineered to express a melanoma-specific T-cell receptor (TCR) against MART-1. Before inoculation of mice with BLM cells, the BLM cells were transduced with the antigen (MART-1) as well as the correct HLA haplotype (HLA-A2) in order for the MART-1-specific T cells to recognize the tumor cells.

Cell Line and Cell Culture Conditions

Melanoma cell line BLM was cultured in DMEM (Gibco), with fetal bovine serum (Sigma), 100 U/mL penicillin (Gibco) and 0.1 mg/mL streptomycin (Gibco) under standard conditions, and was regularly confirmed to be mycoplasma-free by PCR.

HLA-A2 and MART-1 Transduction in BLM

MART-126-35 and HLA-A2 were introduced using lentiviral and retroviral constructs. Constructs for lentivirus were packaged in lentivirus using two helper plasmids (psPax and MS2G, Addgene) in HEK293T cells. Constructs for retrovirus were produced in a packaging cell line (Fly cells). Viral supernatant was either snap frozen or immediately used for infection. MART-126-35-Katushka positive cells were sorted by flow cytometry and seeded into 96-well plates at one cell per well. When single cells grew out, expression of MART-Katushka and HLA-A2 was confirmed by FACS.

BLM Xenograft Model and Treatment

8-14 week old male and female NOD-SCID Gamma (NSGTM) mice (bred in-house at the Netherlands Cancer Institute (NKI), Amsterdam, The Netherlands) were subcutaneously injected in the right flank with 1×106 BLM tumor cells. Tumors were measured three times per week with a caliper, and when tumors were 100 mm3 (after 7 days) the animals were randomized over the following treatment groups:
1. Control T cells+Control ADC (n=7)
2. MART-1 T cells+Control ADC (n=8)
3. MART-1 T cells+Control ADC+anti-PD1 (n=10)
4. Control T cells+IgG1-AXL-107-vcMMAE (n=8)
5. MART-1 T cells+IgG1-AXL-107-vcMMAE (n=8)
6. MART-1 T cells+IgG1-AXL-107-vcMMAE+anti-PD1 (n=9)

On the day of randomization, mice were i.v. injected with a single dose (4 mg/kg) of IgG1-AXL-107-vcMMAE or control ADC (IgG1-b12-vcMMAE). Simultaneously, mice were i.v. injected with MART-1 or control T cells at a dose of 5×106 cells/mouse. The total injected volume was diluted to 200 μL per mouse in PBS. To support the T cells, all mice received intraperitoneally (i.p.) injection with 100.000 IU IL-2 (Proleukin, Novartis; diluted in 100 μL PBS) for 3 consecutive days.

In addition, two groups (groups 3 and 6) received anti-PD1 (pembrolizumab, SelleckChem) weekly via i.p. injection from day 7 onwards, at a dose of 5 mg/kg.

Tumor volumes were measured 3 times per week by an independent animal technician in a blinded fashion. Tumor volume was calculated as follows: length (mm)×width (mm)/2. Tumors were harvested when they reached 1000 mm3. Statistical analysis was performed by Mann-Whitney (one-tailed) comparing AXL-ADC+MART-1 T cells vs. AXL-ADC+MART-1 T cells+anti-PD1 on day 34.

Results

The anti-tumor effect of IgG1-AXL-107-vcMMAE in combination with anti-PD-1 (pembrolizumab) in the BLM human cell line-derived xenograft (CDX) mouse model was assessed in the context of a tumor-specific human T cell response. To this end, the human melanoma cell line BLM was first transduced with an antigen (MART-1) and the correct HLA haplotype (HLA-A2) in order for T cells expressing a TCR against MART-1 to recognize the tumor cells. Subsequently, mice were inoculated with these cells, and after establishment of the xenograft, mice were randomized into different treatment groups (see above), and injected with a single dose of ADC and T cells, while two groups received additional weekly injections of anti-PD1.

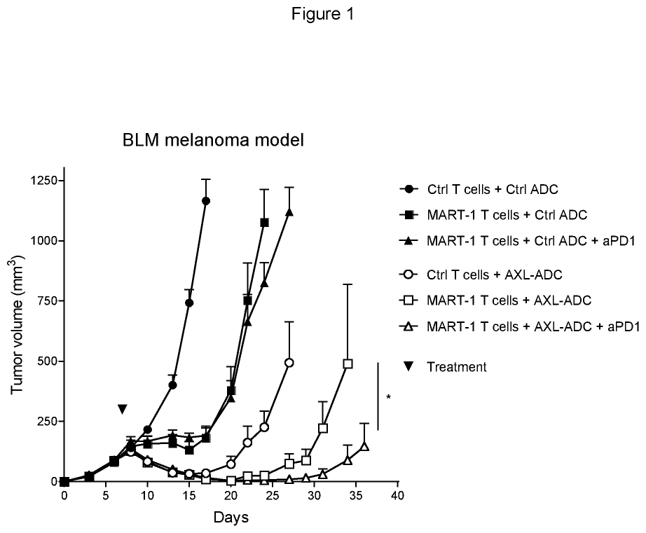

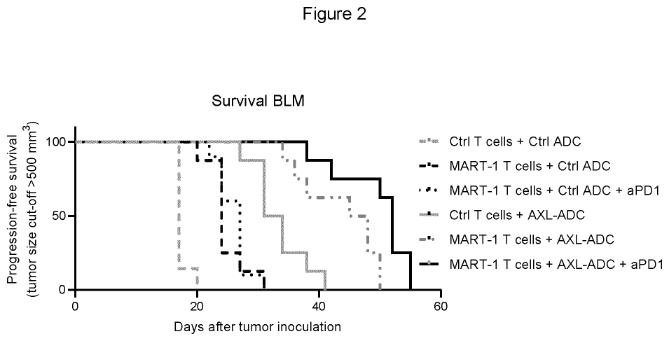

Mice that received tumor antigen-specific T cells (MART-1 T cells) in combination with control ADC showed some tumor growth inhibition compared to mice that received control, non-specific T cells (Ctrl T cells) in combination with control ADC (FIG. 1). However, no enhanced tumor growth inhibition was noted in mice that received anti-PD1 treatment in combination with tumor antigen-specific T cells (MART-1 T cells) and control ADC, indicating that this model is resistant to PD-1/PDL-1 axis inhibition (FIG. 1). In comparison, treatment with IgG1-AXL-107-vcMMAE induced tumor regression after a single dose of 4 mg/kg. This effect was observed in mice that received control T cells, and was further enhanced by the presence of MART-1 T cells. In both instances, treatment with IgG1-AXL-107-vcMMAE led to greater anti-tumor effects compared to tumor antigen-specific T cells alone or in combination with anti-PD1. Importantly, treatment with IgG1-AXL-107-vcMMAE in combination with anti-PD-1 and MART-1 T cells induced more potent tumor regression in comparison to mice that only received IgG1-AXL-107-vcMMAE and MART-1 T cells (FIG. 1; P=0.04). Combination treatment with IgG1-AXL-107-vcMMAE and anti-PD-1 in the context of MART-1 T cells also prolonged the lifespan of these mice compared to all other groups, as indicated by the survival curve (FIG. 2).

These results show that the combination of IgG1-AXL-107-vcMMAE and anti-PD-1 exerts anti-tumor cooperativity and can create de novo sensitization to PD-1 inhibition in a melanoma model refractory to anti-PD-1.

### Example 4—Anti-Tumor Activity of IgG1-AXL-107-vcMMAE in Combination with Anti-PD-1 in the LCLC-103H NSCLC Xenograft Model that is Resistant to Anti-PD-1 Treatment

The anti-tumor activity of IgG1-AXL-107-vcMMAE in combination with anti-PD1 (pembrolizumab) was evaluated in the LCLC-103H NSCLC cell line-derived xenograft model in mice, which systemically received human T-cells that were engineered to express a specific T-cell receptor (TCR) against MART-1. Before inoculation of mice with LCLC-103H cells, the LCLC-103H cells were transduced with the antigen (MART-1) as well as the correct HLA haplotype (HLA-A2) in order for the MART-1-specific T cells to recognize the tumor cells.

Cell Line and Cell Culture Conditions

Melanoma cell line BLM was cultured in DMEM (Gibco), with fetal bovine serum (Sigma), 100 U/mL penicillin (Gibco) and 0.1 mg/mL streptomycin (Gibco) under standard conditions, and was regularly confirmed to be mycoplasma-free by PCR.

HLA-A2 and MART-1 Transduction in BLM

MART-126-35 and HLA-A2 were introduced using lentiviral and retroviral constructs. Constructs for lentivirus were packaged in lentivirus using two helper plasmids (psPax and MS2G, Addgene) in HEK293T cells. Constructs for retrovirus were produced in a packaging cell line (Fly cells). Viral supernatant was either snap frozen or immediately used for infection. MART-126-35-Katushka positive cells were sorted by flow cytometry and seeded into 96-well plates at one cell per well. When single cells grew out, expression of MART-Katushka and HLA-A2 was confirmed by FACS.

LCLC-103H Xenograft Model and Treatment

8-14 week old male and female NOD-SCID Gamma (NSGTM) mice (bred in-house at the Netherlands Cancer Institute (NKI), Amsterdam, The Netherlands) were subcutaneously injected in the right flank with 1×106 BLM tumor cells. Tumors were measured three times per week with a caliper, and when tumors were 50 mm3 (after 16 days), mice were randomized over the following treatment groups:
1. Control T cells+Control ADC (n=7)
2. MART-1 T cells+Control ADC (n=7)
3. MART-1 T cells+Control ADC+anti-PD1 (n=7)
4. Control T cells+IgG1-AXL-107-vcMMAE (n=11)
5. MART-1 T cells+IgG1-AXL-107-vcMMAE (n=11)
6. MART-1 T cells+IgG1-AXL-107-vcMMAE+anti-PD1 (n=11)

From the day of randomization (day 16), mice received weekly i.v. injections of IgG1-AXL-107-vcMMAE or control ADC (IgG1-b12-vcMMAE) at a dos of 1 mg/kg. On day 16 mice received a single i.v. injection with MART-1 or control T cells at a dose of 5×106 cells/mouse. The total injected volume was diluted to 200 μL per mouse in PBS. To support the T cells, all mice received intraperitoneally (i.p.) injection with 100.000 IU IL-2 (Proleukin, Novartis; diluted in 100 μL PBS) for 3 consecutive days starting from day 16.

In addition, two groups (groups 3 and 6) received anti-PD1 (pembrolizumab, SelleckChem) weekly via i.p. injection from day 16 onwards, at a dose of 5 mg/kg.

Tumor volumes were measured 3 times per week by an independent animal technician in a blinded fashion. Tumor volume was calculated as follows: length (mm)×width (mm)/2. Tumors were harvested when they reached 500 mm3. Statistical analysis was performed by Mann-Whitney (one-tailed) comparing AXL-ADC+MART-1 T cells vs. AXL-ADC+MART-1 T cells+anti-PD1 on day 44.

Results

The anti-tumor effect of IgG1-AXL-107-vcMMAE in combination with anti-PD-1 (pembrolizumab) in the LCLC-103H human cell line-derived xenograft (CDX) mouse model was assessed in the context of a tumor-specific human T cell response. To this end, the human NSCLC cell line LCLC-103H was first transduced with an antigen (MART-1) and the correct HLA haplotype (HLA-A2) in order for T cells expressing a TCR against MART-1 to recognize the tumor cells. Subsequently, mice were inoculated with these cells, and after establishment of the xenograft, mice were randomized into different treatment groups (see above), and injected with weekly doses of ADC and a single injection with T cells, while two groups received additional weekly injections of anti-PD1.

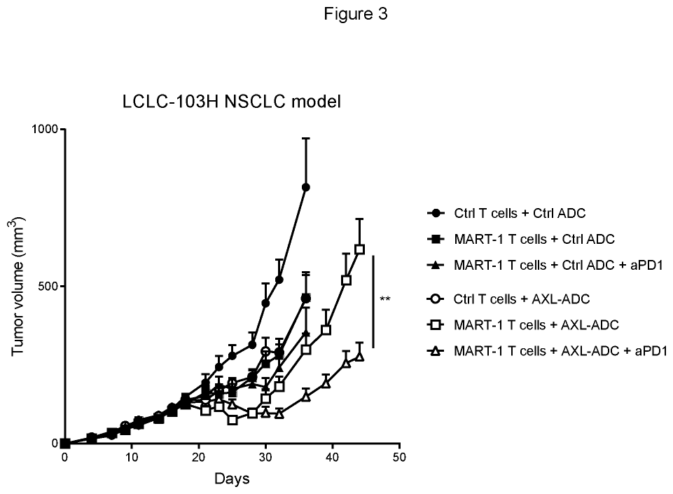

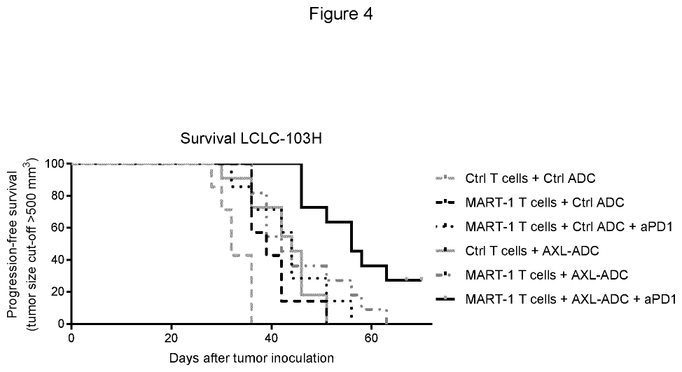

Mice that received antigen-specific T cells (MART-1 T cells) in combination with control ADC showed some tumor growth inhibition compared to mice that received control, non-specific T cells (Ctrl T cells) in combination with control ADC (FIG. 3). However, no enhanced tumor growth inhibition was noted in mice that received anti-PD1 treatment in combination with tumor antigen-specific T cells (MART-1 T cells) and control ADC, indicating that this model is resistant to PD-1/PDL-1 axis inhibition (FIG. 3). Treatment with a low dose of IgG1-AXL-107-vcMMAE (1 mg/kg) induced some tumor growth inhibition in the absence of tumor antigen specific T cells. This effect was observed in mice that received control T cells, and was further enhanced in the setting of MART-1 T cells. Treatment with IgG1-AXL-107-vcMMAE in combination with anti-PD-1 in the presence of MART-1 T cells induced significantly more potent tumor regression in comparison to mice that only received IgG1-AXL-107-vcMMAE and MART-1 T cells (FIG. 3; P=0.006). Combination treatment with IgG1-AXL-107-vcMMAE and anti-PD-1 in the context of MART-1 T cells also significantly prolonged the lifespan of these mice compared to all other groups, as indicated by the survival curve (FIG. 4).

These results show that combination of IgG1-AXL-107-vcMMAE and anti-PD-1 in the presence of tumor antigen-specific T cells is more efficacious than treatment with IgG1-AXL-107-vcMMAE or anti-PD-1 alone in the LCLC-103H human NSCLC model, consistent with anti-tumor cooperativity between Axl-targeting ADCs and PD-1 inhibition in NSCLC.

### Example 5—IgG1-AXL-107-vcMMAE Induces an Inflammatory Response in PDX of Lung Cancer In Vivo

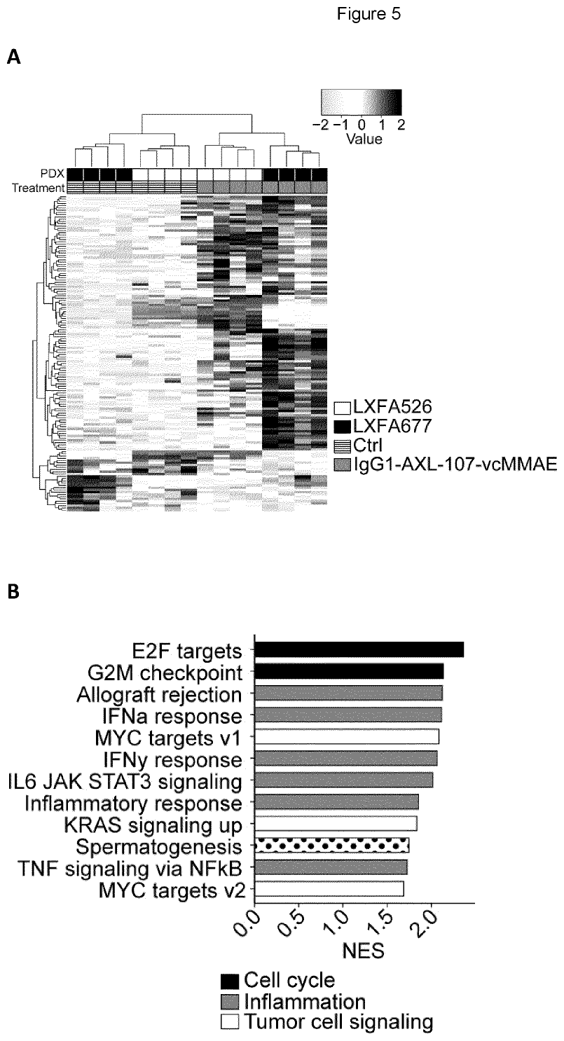

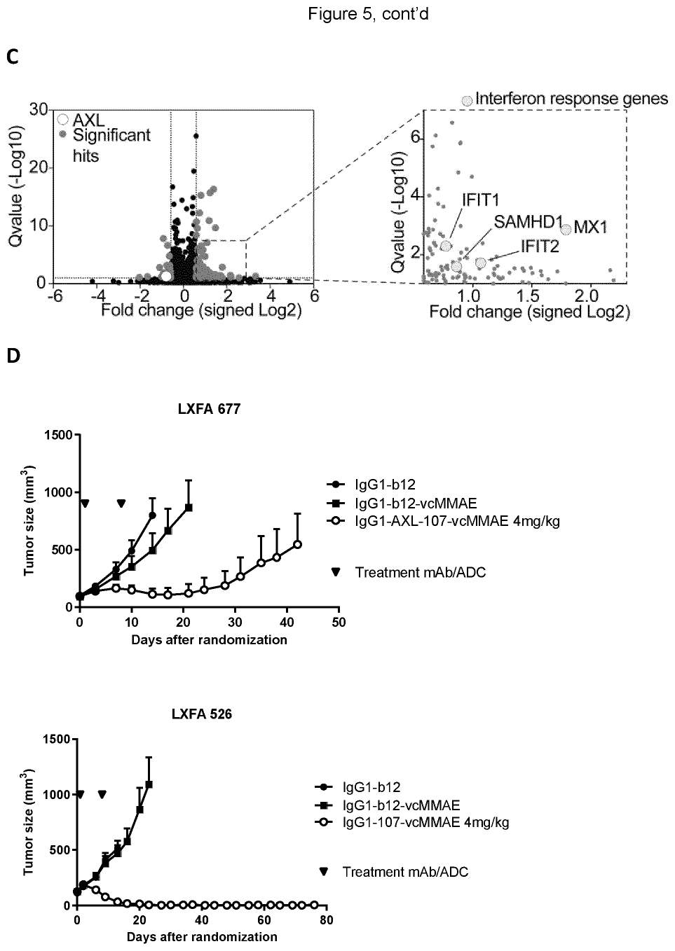

Two AXL-positive lung cancer PDX were implanted into mice and, after tumor establishment, treated with either IgG1-AXL-107-vcMMAE or IgG1-1312 control antibody see FIG. 5D for long-term sensitivity to IgG1-AXL-107-vcMMAE in these models). Next, tumors were harvested 3 or 6 days after treatment, and evaluated for mRNA and protein expression. Principal component analysis (PCA) of the genes differentially modulated by IgG1-AXL-107-vcMMAE treatment revealed a principal component (PC3) that separated IgG1-AXL-107-vcMMAE treated tumors from control tumors in both PDX models (FIG. 5A). Gene set enrichment analysis (GSEA) of PC3 showed that the highest enriched gene sets were involved in cell cycle checkpoints (FIG. 5B). This is consistent with the mechanism of action of MMAE, the cytotoxic moiety linked to the AXL antibody of IgG1-AXL-107-vcMMAE, which causes G2/M cell cycle arrest. Other top enriched gene sets were dominated by inflammatory processes, such as interferon response pathways (FIG. 5B). These results suggested that IgG1-AXL-107-vcMMAE causes inflammation responses in tumor cells. This was further corroborated by proteomic profiling of the tumor cells, which showed that several inflammatory genes were induced upon IgG1-AXL-107-vcMMAE treatment (FIG. 5C).

### Example 6—IgG1-AXL-107-vcMMAE Promotes an Inflammatory Tumor Phenotype in an AXL-Positive Lung Cancer PDX Model in HIS Mice

An AXL-positive lung cancer PDX model was implanted into 21 week old female huCD34 NSG-SGM3 mice with a humanized immune system (HIS). HIS mice are able to partly reconstitute the human immune system derived from transplantation of human CD34+ hematopoietic stem cells; despite the humanized immune system, the HIS mice are still permissive of tumor xenografts.

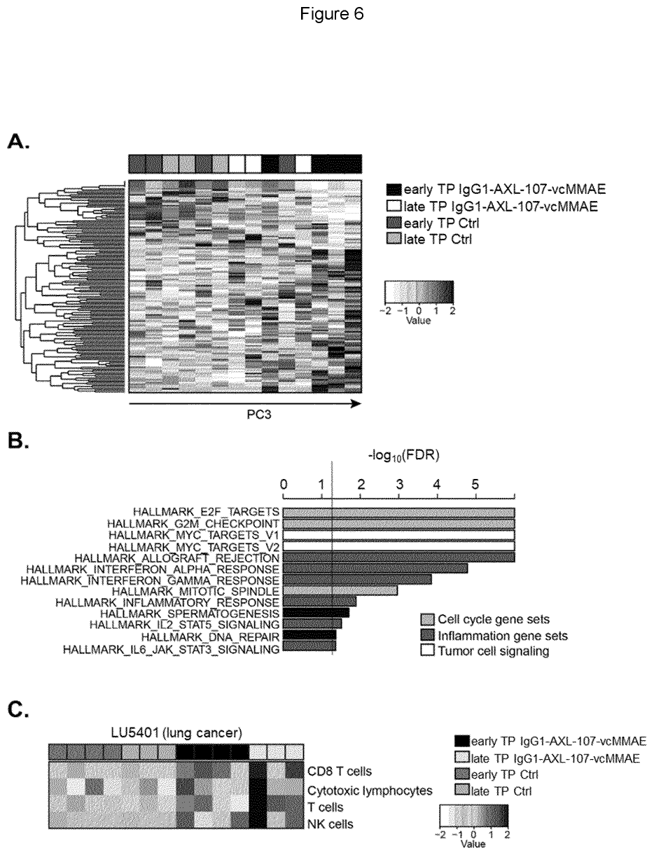

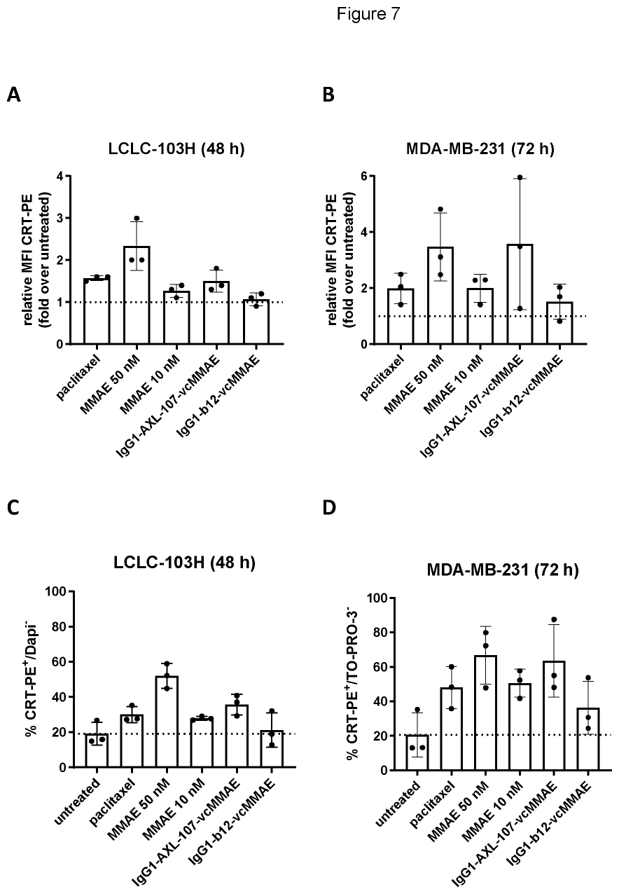

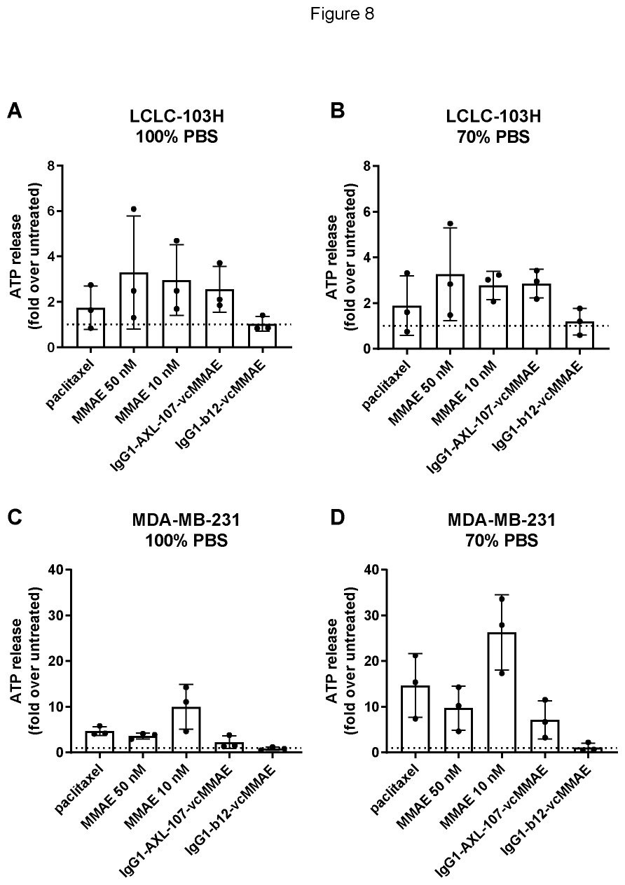

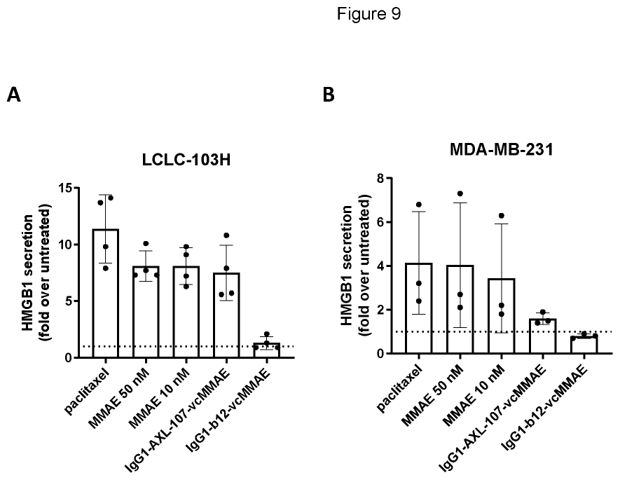

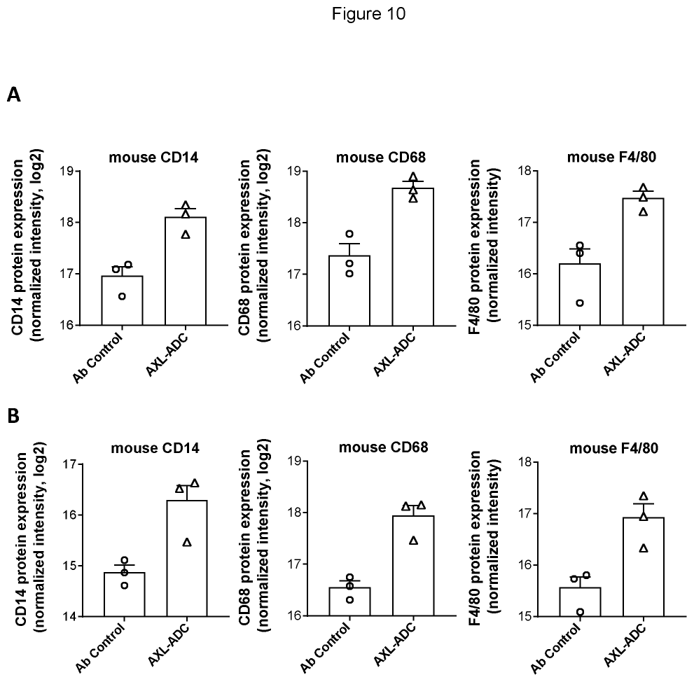

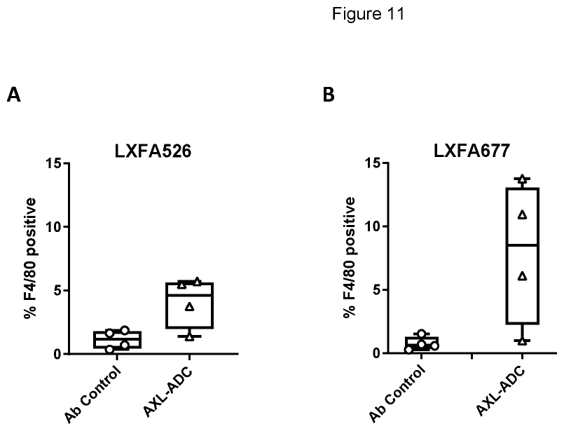

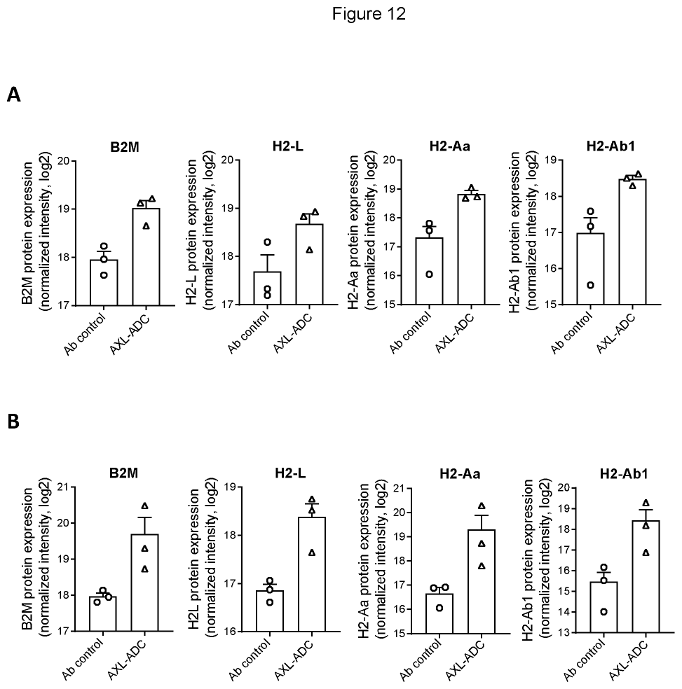

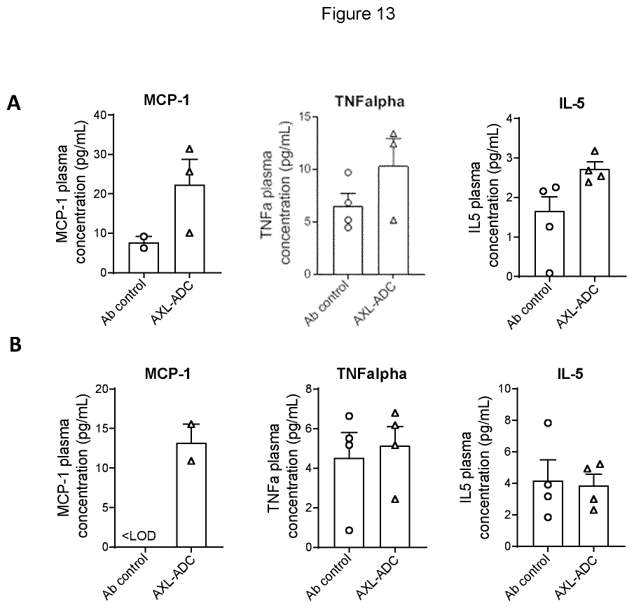

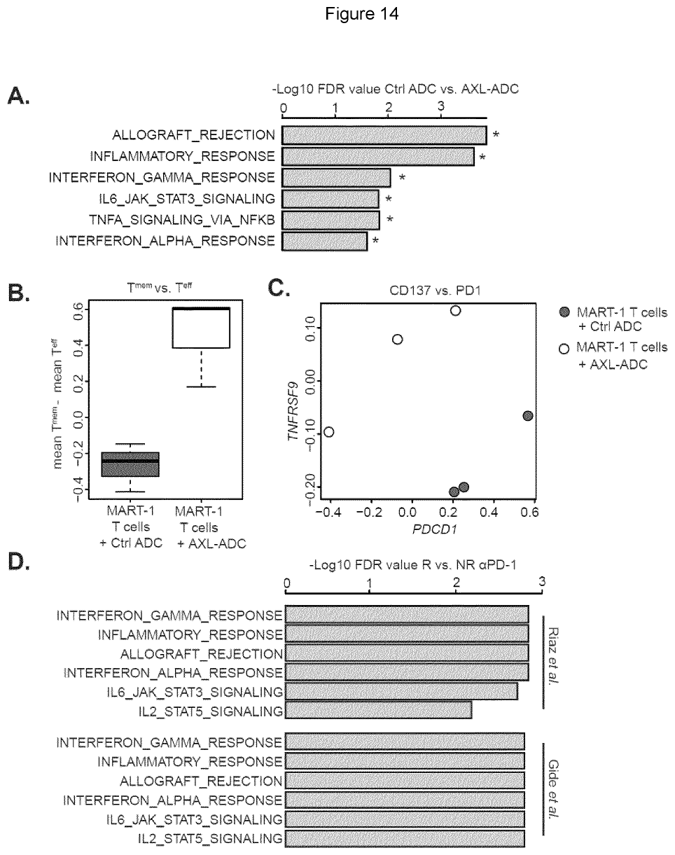

After tumor establishment (average tumor volume per treatment group 355-428 mm3), mice were treated weekly with either 4 mg/kg IgG1-AXL-107-vcMMAE or IgG1-b12 for up to two times. Next, tumors were harvested 3 days after treatment start or within an average of 30 days (range 28-35 days) after initiation of the treatment. The sampled tumors were evaluated for mRNA expression by RNA sequencing. In concordance with the molecular profiling results of the PDX models described in example 5, principal component analysis (PCA) of the gene expression results revealed a principal component (PC3) able to separate to a large extent IgG1-AXL-107-vcMMAE treated tumors from control tumors (FIG. 6A). Gene set enrichment analysis (GSEA) of PC3 showed that the highest enriched gene sets were involved in cell cycle checkpoints (FIG. 6B). This is consistent with the mechanism of action of MMAE, the cytotoxic moiety linked to the AXL antibody of IgG1-AXL-107-vcMMAE, which causes G2/M cell cycle arrest. Other top enriched gene sets were predominantly associated with inflammatory processes, such as interferon response pathways and cytokine signaling (FIG. 6B). These results suggest that IgG1-AXL-107-vcMMAE causes an inflammation response in tumor cells. This was further supported by the observation that gene signatures of tumor-infiltrating lymphocytes (TILs), including cytotoxic TILs, were enriched in tumors of IgG1-AXL-107-vcMMAE treated mice compared to the IgG1-b12 treated control group (FIG. 6C).

### Example 7—IgG1-AXL-107-vcMMAE Induces Damage-Associated Molecular Patterns (DAMPs) In Vitro

Auristatin payloads, including monomethyl auristatin E (MMAE), disrupt the microtubule networks resulting in cellular stress, including mitotic arrest and ER stress, culminating in a phenotype known as immunogenic cell death (ICD). ICD is a mode of cell death that generates immune responses against the dying cells. Characteristic for ICD is that molecules that are normally found within cells become exposed on the cell surface or are secreted, leading to recognition by innate immune cells. This in turn leads to increased antigen presentation and activation of the adaptive immune response. The ability of cytostatic agents to induce ICD provides a mechanistic rationale for anti-tumor cooperativity with PD1-/PD-L1 inhibitors. To this end, it was evaluated whether treatment of lung and breast cancer cell lines with IgG1-AXL-107-vcMMAE induces hallmarks of ICD in vitro, including calreticulin exposure on the cell surface, and extracellular release of ATP and HMGB1. These hallmarks of ICD are also known as damage-associated molecular patterns (DAMPs).

Calreticulin (CRT) is a protein that is normally localized within the endoplasmic reticulum (ER); its exposure at the cell surface of stressed and dying cells is characteristic for ICD. In addition, cells release ATP early during apoptosis, which constitutes a ‘find’me’ signal for the recruitment of dendritic cells (DCs) and their precursors as well as a pro-inflammatory stimulus to activate NOD-like receptors on DCs and macrophages. Furthermore, the ICD hallmark protein amphoterin (or nonhistone chromatin protein high-mobility group box 1 [HMGB1]) resides normally inside the cell nucleus but it is actively secreted by cells undergoing severe stress or cell death, and also operates as a pro-inflammatory stimulus. Together, these DAMPs enable the immune system to recognize and mount cytotoxic activity against tumor cells.

Evaluation of Calreticulin Surface Expression In Vitro

LCLC-103H or MDA-MB-231 cells were grown to 70% to 80% confluence in 6-well plates, washed with serum-free medium and incubated with IgG1-AXL-107-vcMMAE (2 μg/mL), free monomethyl auristatin E (MMAE, SAFC, cat.no SG10; 10 or 50 nM), paclitaxel (1 μM), or isotype control ADC (IgG1-b12-vcMMAE; 2 μg/mL) in serum-free medium (RPMI1640, Lonza BE17-603E) for 48 hours (LCLC-103H) or 72 hours (MDA-MB-231). After incubation, supernatant and cells were collected and washed in PBS/0.1% BSA/0.02% azide (FACS buffer). Cells were then incubated with a phycoerythrin (PE)-conjugated mouse anti-human calreticulin antibody (Enzo, Cat no. ADI-SPA-601PE-D, lot. 10021835) for 30 min at 4° C. in the dark. A PE-conjugated mouse isotype control antibody (Enzo, cat no. ADI-SAB-600PE-D, lot. 08071817) was included as a control. After washing with FACS buffer, cells were analyzed with flow cytometry in the presence of DAPI (BD; cat no. 564907, lot. 8012653) or TO-PRO-3 (Thermo Fisher, cat no. T3605, lot. 1976612). Calreticulin expression was determined as PE positive cells and the percentage of calreticulin positive cells over isotype control was obtained on DAPI or TO-PRO-3 negative cells.

FIG. 7 shows that treatment with IgG1-AXL-107-vcMMAE results in increased cell surface expression of calreticulin in LCLC-103H (on average about 1.4-fold after 48 h) and MDA-MB-231 cells (on average about 3.5-fold after 72 h) compared to untreated cells. The increased levels of calreticulin expression were comparable to calreticulin surface expression levels induced by paclitaxel or MMAE, which were included as positive control treatments. In contrast, cells treated with isotype control ADC did not show upregulated calreticulin surface expression (FIGS. 3A and B). The percentage of calreticulin positive cells also increased upon treatment with IgG1-AXL-107-vcMMAE, free MMAE, or paclitaxel compared to untreated or isotype control ADC treatment in in both cell lines (FIGS. 3C and D).

Evaluation of Extracellular ATP Release In Vitro

LCLC-103H or MDA-MB-231 cells were seeded in quadruplicate at 25,000 cells per well for LCLC-103H cells or 125,000 cells per well for MDA-MB-231 cells in 24-well plates and grown 3 hours at 37° C. Subsequently, IgG1-AXL-107-vcMMAE (2 μg/mL), free monomethyl auristatin E (MMAE; 10 or 50 nM), paclitaxel (1 μM), or isotype control ADC (IgG1-b12-vcMMAE; 2 μg/mL) were added per well.

Plates were incubated for 48 hr at 37° C. and after incubation each well was washed with 200 μL PBS. After the final wash, PBS was replaced with 200 μL fresh 100% PBS or 70% PBS (hypotonic) for the indicated times, harvested and transferred to a 96-well plate, and then spun at 1000 rpm for 2 min at RT. Supernatants were transferred to a new 96-well (optiwhite) plate and ATP was measured using CellTiterGlo Luminescent Cell Viability Assay (Promega, Cat.no G7570) according to manufacturer's instructions. All steps were performed with ice-cold PBS and in the dark.

FIG. 8A shows that treatment with IgG1-AXL-107-vcMMAE results in about 3-fold increase of ATP release in LCLC-103H cells compared to untreated cells or cells treated with isotype control ADC. LCLC-103H cells treated with paclitaxel or free MMAE showed also release of extracellular ATP at comparable levels as induced by IgG1-AXL-107-vcMMAE. Under hypotonic conditions (70% PBS), which may further stimulate extracellular release of ATP, similar results were obtained for LCLC-103H cells (FIG. 8B). In MDA-MB-231 cells, a 2- to 5-fold increase of ATP release was observed upon treatment with IgG1-AXL-107-vcMMAE, free MMAE (50 nM), or paclitaxel compared to untreated cells or cells treated with isotype control ADC, while cells treated with 10 nM MMAE showed about 10-fold increased ATP release compared to untreated cells (FIG. 8C). In MDA-MB-231 cells under hypotonic conditions, more pronounced ATP release was observed upon treatment with IgG1-AXL-107-vcMMAE (^{˜}7-fold), 50 nM free MMAE (^{˜}10-fold), paclitaxel (^{˜}15-fold), or 10 nM free MMAE (^{˜}26-fold) compared to untreated cells (FIG. 8D). The isotype control ADC did not induce enhanced levels of extracellular ATP under these conditions.

Evaluation of HGMB1 Secretion In Vitro

MDA-MB-231 or LCLC-103H cells were seeded in quadruplicate at 125,000 cells per well (MDA-MB-231) or 25,000 cells/well (LCLC-103H) in 24-well plates and grown 3-4 hrs at 37° C. Subsequently, IgG1-AXL-107-vcMMAE (2 μg/mL), free monomethyl auristatin E (MMAE; 10 or 50 nM), paclitaxel (1 μM), or isotype control ADC (IgG1-b12-vcMMAE; 2 μg/mL) were added to the wells and plates were incubated for 48 hrs at 37° C. After incubation cells were pelleted at 1000 rpm for 3 min at RT and supernatants were transferred to a new 96-wells plate. HMGB1 was measured in the supernatant using a HMGB1-specific ELISA kit (IBL, Cat.no ST51011) according to manufacturer's instructions.

FIG. 9A shows that treatment with IgG1-AXL-107-vcMMAE results in about 7.5-fold increase of HMGB1 secretion by LCLC-103H cells compared to untreated cells or cells treated with isotype control ADC. These levels were similar in cells treated with free MMAE (10 or 50 nM), while paclitaxel induced somewhat more HMGB1 secretion: about 11-fold over untreated control. In MDA-MB-231 cells, less pronounced secretion of HMGB1 was observed: treatment with IgG1-AXL-107-vcMMAE resulted in about 1.6-fold increase of HMGB1 secreted by these cells, while paclitaxel and free MMAE induced on average around 4-fold higher levels of extracellular HMGB1 in MDA-MB-231 (FIG. 9B).

Together, these results demonstrate that treatment of tumor cell lines with IgG1-AXL-107-vcMMAE induced important hallmarks of ICD in vitro, including calreticulin exposure on the cell surface, extracellular release of ATP, and secretion of HMGB1. This may generate immune responses against the dying cells and provides a rationale to combine IgG1-AXL-107-vcMMAE treatment with immune checkpoint blockers, such as anti-PD-1 antibodies, to cooperatively boost the anti-tumor immune response.

### Example 8—IgG1-AXL-107-vcMMAE Mediates Tumor Influx of Innate Immune Cells, Leading to Increased Antigen Presentation in Lung Cancer Models In Vivo

The effect of IgG1-AXL-107-vcMMAE treatment on innate immune cells was evaluated in subcutaneous patient-derived xenograft (PDX) models of NSCLC (LXFA 526 and LXFA 677) in NMRI nu/nu mice. These mice are deficient of T cells, but do have innate immune cells such as dendritic cells, monocytes, and macrophages.

PDX Models

Tumor fragments from donor mice bearing patient-derived NSCLC xenografts (LXFA 526 or LXFA 677) were used for inoculation of 4-6 weeks old male NMRI nu/nu mice (experiments performed by Oncotest, Freiburg, Germany). Randomization of animals was performed as follows: animals bearing a tumor with a volume of about 200 mm3 were distributed in 7 experimental groups (3 animals per group), considering a comparable median and mean of group tumor volume. The treatment groups were:
1. IgG-b12, t=0
2. IgG-b12, t=2d
3. IgG-b12, t=3d
4. IgG-b12, t=6d
5. IgG1-AXL-107-vcMMAE, t=2d
6. IgG1-AXL-107-vcMMAE, t=3d
7. IgG1-AXL-107-vcMMAE, t=6d

IgG1-b12 and IgG1-AXL-107-vcMMAE were dosed at 4 mg/kg and were intravenously (i.v.) injected on the day of randomization (day 0). At these doses, it has been shown that IgG1-AXL-107-vcMMAE has strong anti-tumor activity in the LXFA 526 and LXFA 677 models, while IgG1-b12 did not affect tumor growth in these models (Boshuizen et al, Nature Med 2018). At the indicated time points, the mice were terminated and tumor and plasma samples were collected for molecular profiling. Formalin-fixed, paraffin-embedded (FFPE) tumor samples were analyzed by immunohistochemistry for F4/80 macrophage marker expression and quantified using digital pathology. Fresh frozen tumor samples were subjected to tissue lysis, protein extraction and trypsin digestion. Isolated peptides were then quantified and used for construction of sample-specific spectral library using shotgun LC-MS/MS technology; samples were then analyzed by high-reaction monitoring (HRM™) mass-spectroscopy for the unbiased detection of all detectable proteins and peptides (Biognosys, Schlieren, Switzerland). The plasma samples were analyzed for mouse cytokine expression using the ProCartaPlex immunoassay kit which measures 17 cytokines based on the Luminex technology (ThermoFisher Scientific, Waltham, Mass. USA).

Results

FIG. 10 shows that treatment with IgG1-AXL-107-vcMMAE resulted in increased tumor expression of the monocyte/macrophage-associated proteins CD14, CD68, and F4/80 in in the LXFA-526 (FIG. 10A) and LXFA-677 (FIG. 10B) models after 6 days, as evaluated by proteomic profiling. Immunohistochemistry confirmed enhanced tumor influx of F4/80-expressing cells upon IgG1-AXL-107-vcMMAE treatment both lung PDX models after 6 days (FIG. 11).

Consistent with the recruitment of antigen-presenting cells by IgG1-AXL-107-vcMMAE, increased expression of mouse H2 class I (H2-L and B2M) and class II (H2-Aa and H2-Ab1) proteins was noted in the LXFA-526 (FIG. 12A) and LXFA-677 (FIG. 12B) models 6 days after treatment.

Mouse plasma cytokines levels were evaluated, and measured levels showed large variability and were very low for the majority of cytokines. MCP-1, TNFalpha, and IL-5 were present at detectable levels and increased upon treatment with IgG1-AXL-107-vcMMAE in the plasma of the LXFA-526 model after 3 days (FIG. 13A). In the LXFA-677 model, MCP-1 was not detectable in the plasma of control mice, but was induced in mice that received IgG1-AXL-107-vcMMAE (FIG. 13B). TNFalpha and IL-5 were present at detectable levels in the plasma of control mice of the LXFA-677 model, but levels were not different in mice treated with IgG1-AXL-107-vcMMAE (FIG. 13B).

Together, these data show that treatment with IgG1-AXL-107-vcMMAE enhanced tumor influx of host innate immune cells and increased antigen presentation and cytokine release, suggesting that IgG1-AXL-107-vcMMAE has immunogenic effects that could enhance T-cell-mediated immunity, providing mechanistic rationale for the anti-tumor cooperativity seen with anti-PD-1. As IgG1-AXL-107-vcMMAE does not bind to mouse Axl, the observed effects on host immune cells are MMAE-dependent and may be the result of DAMPs exposed and/or secreted by the tumor in response to IgG1-AXL-107-vcMMAE.

### Example 9—EnaV Promotes an Inflammatory Tumor Phenotype and Induces a Memory-Like State in T Cells

It was evaluated whether IgG1-AXL-107-vcMMAE treatment induced an inflammatory response in the human melanoma BLM cell line-derived xenograft mouse model co-injected with human, tumor-specific T-cells.

BLM Xenograft Model and Treatment

8-14 week old male and female NOD-SCID Gamma (NSGTM) mice (bred in-house at the Netherlands Cancer Institute (NKI), Amsterdam, The Netherlands) were subcutaneously injected in the right flank with 1×106 BLM tumor cells. Tumors were measured three times per week with a caliper, and when tumor volume reached approximately 100 mm3 (after 7 days) the animals were randomized over the following treatment groups:
1. Control T cells+Control ADC (n=3)
2. MART-1 T cells+Control ADC (n=3)
3. Control T cells+IgG1-AXL-107-vcMMAE (n=3)
4. MART-1 T cells+IgG1-AXL-107-vcMMAE (n=3)

On the day of randomization, mice were i.v. injected with a single dose (4 mg/kg) of IgG1-AXL-107-vcMMAE or control ADC (IgG1-b12-vcMMAE). Simultaneously, mice were i.v. injected with MART-1 or control T cells at a dose of 5×106 cells/mouse. The total injected volume was diluted to 200 μL per mouse in PBS. To support the T cells, all mice received intraperitoneally (i.p.) injection with 100.000 IU IL-2 (Proleukin, Novartis; diluted in 100 μL PBS) for 3 consecutive days.

On Day 7 after the start of treatment, the tumors were harvested, and RNA sequencing was performed on the tissues.

The human RNA reads were first computationally dissected from the mouse reads by XenofilteR to investigate the human tumor cell and (transduced) T cell compartments. Geneset enrichment analysis (GSEA) was performed using the BROAD javaGSEA standalone version (http://www.broadinstitute.org/gsea/downloads.jsp). Analysis was run using 10,000 permutations. GSEA showed significant induction of inflammation-associated Hallmark gene sets of the MSigDB collection in IgG1-AXL-107-vcMMAE treated tissues compared to controls (FIG. 14 A).

Furthermore, using publically available datasets (GSE10239) we found that the infiltrated T-cells were skewed towards a memory-like phenotype in IgG1-AXL-107-vcMMAE-treated samples, while the T-cells in the controls showed a classical effector T-cell phenotype (FIG. 14 B).

In addition, in IgG1-AXL-107-vcMMAE treated tissues, T cells showed increased RNA expression of the activation marker CD137 (TNFRSF9) and reduced PD-1 expression (FIG. 14 C), suggesting an increased T cell activation status.

Next, geneset enrichment analysis was performed on publicly available, clinical transcriptome datasets for melanoma (Riaz, N. et al. Cell 171, 934-949.e16 (2017); Gide, T. N. et al. Cancer Cell 35, 238-255.e6 (2019)). Of interest, the inflammatory genesets which were significantly enriched in the preclinical models upon treatment with IgG1-AXL-107-vcMMAE, were able to differentiate between responders (R) versus non-responding (NR) patients to anti-PD-1 immunotherapy in the two independent clinical datasets (FIG. 14 D). This suggests that an inflammatory microenvironment, such as induced by IgG1-AXL-107-vcMMAE treatment, may be beneficial for response to immune checkpoint blockers.

1. A conjugate of a cytotoxic agent and an antibody capable of binding to human Axl (e.g. human Axl having the sequence set forth in SEQ ID NO: 1) for use in potentiating the therapeutic efficacy of programmed cell death-1 (PD-1) and/or programmed death-ligand 1 (PD-L1) inhibition in a subject, by inducing immunogenic cell death and/or tumor-associated inflammation; e.g. tumor-associated inflammation associated with immunogenic cell death.

2. The conjugate for use according to claim 1, wherein the conjugate is used in combination with an inhibitor of PD-1 and/or PD-L1.

3. A conjugate of a cytotoxic agent and an antibody capable of binding to human Axl (e.g. human Axl having the sequence set forth in SEQ ID NO: 1) for use in treating cancer in a subject, in combination with an inhibitor of programmed cell death-1 (PD-1) and/or programmed death-ligand 1 (PD-L1).

4. The conjugate for use according to any one of the preceding claims, wherein the antibody does not compete for AXL binding with the ligand Growth Arrest-Specific 6 (Gas6).

5. The conjugate for use according to any one of the preceding claims, wherein maximal binding of the antibody to AXL in the presence of Gas6 is at least 90%, such as at least 95%, such as at least 97%, such as at least 99%, such as 100%, of binding in the absence of Gas6 as determined by a competition assay, wherein competition between said antibody or antigen-binding fragment and Gas6 is determined on A431 cells preincubated with Gas6 and without Gas6.

6. The conjugate for use according to any one of the preceding claims, wherein the antibody thereof has a binding affinity (KD) in the range of 0.3×10⁻⁹ to 63×10⁻⁹ M to human AXL, and wherein said binding affinity is measured using a Bio-layer Interferometry using soluble AXL extracellular domain.

7. The conjugate for use according to any one of the preceding claims, wherein the antibody has a dissociation rate of 9.7×10⁻⁵ to 4.4×10⁻³ s⁻¹ to AXL, and wherein the dissociation rate is measured by Bio-layer Interferometry using soluble recombinant AXL extracellular domain.

8. The conjugate for use according to any one of the preceding claims, wherein the antibody is capable of being internalized when cell surface bound.

9. The conjugate for use according to claim 8, wherein internalization is determined by a procedure comprising the steps of:
i) Seeding cells, such as MDA-MB-231 or Calu-1 cells (human lung carcinoma cell line; ATCC, catalognumber HTB-54), in 96-well tissue culture plates, 50.000 cells per well, and allowing the cells to attach for 6 hrs at 37° C.,
ii) Incubating the cells in tissue culture medium with serial dilutions of AXL-antibodies (final concentration range 0.0032-10 μg/mL) at 4° C. for 1 hour,
iii) Replacing the tissue culture medium with antibody by tissue culture medium without antibody and incubating the cells overnight (16-18 hours) at 37° C. or 4° C.
iv) Detaching the cells with 40 μL warm trypsin solution, and washing the cells with ice-cold PBS/0.1% BSA/0.02% azide, and incubating the cells for 30 minutes at 4° C. with R-Phycoerythrin (PE)-conjugated goat-anti-human IgG F(ab′)2 (Jackson ImmunoResearch Laboratories, Inc., West Grove, Pa.; cat. No. 109-116-098) diluted 1/100 in PBS/0.1% BSA/0.02% azide (final volume 100 μL), washing the cells were twice in PBS/0.1% BSA/0.02% azide, resuspending the cells in 120 μL PBS/0.1% BSA/0.02% azide,
v) Analyzing the cells by flow cytometry, wherein binding curves are analyzed using non-linear regression (sigmoidal dose-response with variable slope), optionally using Graph Pad Prism V5.04 or later software (Graph Pad Software, San Diego, Calif., USA).

10. The conjugate for use according to any one of the preceding claims, wherein
a) the antibody binds to an epitope within the Ig-like domain I (Ig1) domain of human Axl,
b) the antibody binds to an epitope which comprises or requires one or more amino acids corresponding to positions L121 to Q129 or T112 to Q124 of human AXL having the sequence set forth in SEQ ID NO: 1, and/or
c) binding of the antibody to a chimeric Axl molecule as set forth in SEQ ID NO: 131 is reduced, such as by at least 50% compared to binding of the antibody to human Axl having the sequence set forth in SEQ ID NO: 1, when binding is determined as described in Example 3 of WO 2016/005593.

11. The conjugate for use according to any one of claims 1-9, wherein
a) the antibody binds to an epitope within the Ig-like domain II (Ig2) of human AXL,
b) the antibody binds to an epitope which comprises or requires the amino acids corresponding to position D170 to R190 or the combination of D179 and one or more amino acids corresponding to positions T182 to R190 of human AXL having the sequence set forth in SEQ ID NO: 1, and/or
c) binding of the antibody to a chimeric Axl molecule as set forth in SEQ ID NO: 132 is reduced, such as by at least 50% compared to binding of the antibody to human Axl having the sequence set forth in SEQ ID NO: 1, when binding is determined as described in Example 3 of WO 2016/005593.

12. The conjugate for use according to any one of claims 1-9, wherein
a) the antibody binds to an epitope within the FNIII-like domain I (FN1) of human AXL,
b) the antibody binds to an epitope, which comprises or requires one or more amino acids corresponding to positions Q272 to A287 and G297 to P301 of human AXL of human AXL having the sequence set forth in SEQ ID NO: 1, and/or
c) binding of the antibody to a chimeric Axl molecule as set forth in SEQ ID NO: 133 is reduced, such as by at least 50% compared to binding of the antibody to human Axl having the sequence set forth in SEQ ID NO: 1, when binding is determined as described in Example 3 of WO 2016/005593.

13. The conjugate for use according to any one of claims 1-9, wherein
a) the antibody binds to an epitope within the FNIII-like domain II (FN2) of human AXL,
b) the antibody binds to an epitope which comprises or requires the amino acids corresponding to positions A359, R386, and one or more amino acids corresponding to positions Q436 to K439 of human AXL having the sequence set forth in SEQ ID NO: 1, and/or
c) binding of the antibody to a chimeric Axl molecule as set forth in SEQ ID NO: 134 is reduced, such as by at least 50% compared to binding of the antibody to human Axl having the sequence set forth in SEQ ID NO: 1, when binding is determined as described in Example 3 of WO 2016/005593.

14. The conjugate for use according to any one of the preceding claims, wherein binding of the antibody to Axl, such as human and/or chimeric Axl, is determined by a procedure comprising the steps of
I) providing cells, such as HEK293T cells, transiently transfected with an expression construct for human Axl having the sequence set forth in SEQ ID NO: 130, or an expression construct for a chimeric Axl molecule as set forth in any one of SEQ ID NOs: 131 to 134;
II) incubating the cells with serial dilutions of said antibody with a final concentration range of 0.0024-10 μg/mL for 30 minutes at 4° C.;
III) washing three times in PBS/0.1% BSA/0.02% azide, incubating the cells with R-Phycoerythrin (PE)-conjugated goat-anti-human IgG F(ab′)2 diluted 1/100 in PBS/0.1% BSA/0.02% azide, washing the cells twice in PBS/0.1% BSA/0.02% azide, and resuspending the cells in 120 μL PBS/0.1% BSA/0.02% azide; and
IV) analyzing the cells by flow cytometry.

15. The conjugate for use according to any one of the preceding claims, wherein the antibody comprises at least one binding region comprising a variable heavy chain (VH) region and a variable light chain (VL) region selected from the group consisting of:
a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148];
c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733];
d) a VH region comprising the CDR1, CDR2, and CDR3 sequences of SEQ ID Nos.: 51, 52, and 53, respectively; and a VL region comprising the CDR1, CDR2, and CDR3 sequences of SEQ ID Nos.: 55, GAS, and 56, respectively [154];
e) a VH region comprising the CDR1, CDR2, and CDR3 sequences of SEQ ID Nos.: 51, 52, and 54, respectively; and a VL region comprising the CDR1, CDR2, and CDR3 sequences of SEQ ID Nos.: 55, GAS, and 56, respectively [154-M103L];
f) a VH region comprising the CDR1, CDR2, and CDR3 sequences of SEQ ID Nos.: 57, 58, and 59, respectively; and a VL region comprising the CDR1, CDR2, and CDR3 sequences of SEQ ID Nos.: 60, GAS, and 61, respectively, [171];
g) a VH region comprising the CDR1, CDR2, and CDR3 sequences of SEQ ID Nos.: 62, 63, and 64, respectively; and a VL region comprising the CDR1, CDR2, and CDR3 sequences of SEQ ID Nos.: 65, GAS, and 66, respectively, [172];
h) a VH region comprising the CDR1, CDR2, and CDR3 sequences of SEQ ID Nos.: 67, 68, and 69, respectively; and a VL region comprising the CDR1, CDR2, and CDR3 sequences of SEQ ID Nos.: 70, GAS, and 71, respectively, [181];
i) a VH region comprising the CDR1, CDR2, and CDR3 sequences of SEQ ID Nos.: 72, 73, and 75, respectively; and a VL region comprising the CDR1, CDR2, and CDR3 sequences of SEQ ID Nos.: 76, ATS, and 77, respectively, [183];
j) a VH region comprising the CDR1, CDR2, and CDR3 sequences of SEQ ID Nos.: 72, 74, and 75, respectively; and a VL region comprising the CDR1, CDR2, and CDR3 sequences of SEQ ID Nos.: 76, ATS, and 77, respectively, [183-N52Q];
k) a VH region comprising the CDR1, CDR2, and CDR3 sequences of SEQ ID Nos.: 78, 79, and 80, respectively; and a VL region comprising the CDR1, CDR2, and CDR3 sequences of SEQ ID Nos.: 81, AAS, and 82, respectively, [187];
l) a VH region comprising the CDR1, CDR2, and CDR3 sequences of SEQ ID Nos.: 83, 84, and 85, respectively; and a VL region comprising the CDR1, CDR2, and CDR3 sequences of SEQ ID Nos.: 86, GAS, and 87, respectively, [608-01];
m) a VH region comprising the CDR1, CDR2, and CDR3 sequences of SEQ ID Nos.: 88, 89, and 90, respectively; and a VL region comprising the CDR1, CDR2, and CDR3 sequences of SEQ ID Nos.: 9, GAS, and 92, respectively, [610-01];
n) a VH region comprising the CDR1, CDR2, and CDR3 sequences of SEQ ID Nos.: 93, 94, and 95, respectively; and a VL region comprising the CDR1, CDR2, and CDR3 sequences of SEQ ID Nos.: 96, GAS, and 97, respectively, [613];
o) a VH region comprising the CDR1, CDR2, and CDR3 sequences of SEQ ID Nos.: 98, 99, and 100, respectively; and a VL region comprising the CDR1, CDR2, and CDR3 sequences of SEQ ID Nos.: 101, DAS, and 102, respectively, [613-08];
p) a VH region comprising the CDR1, CDR2, and CDR3 sequences of SEQ ID Nos.: 103, 104, and 105, respectively; and a VL region comprising the CDR1, CDR2, and CDR3 sequences of SEQ ID Nos.: 106, GAS, and 107, respectively, [620-06];
q) a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively, [726];
r) a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively, [726-M101L];
s) a VH region comprising the CDR1, CDR2, and CDR3 sequences of SEQ ID Nos.: 41, 42, and 43, respectively; and a VL region comprising the CDR1, CDR2, and CDR3 sequences of SEQ ID Nos.: 44, AAS, and 45, respectively, [140];
t) a VH region comprising the CDR1, CDR2, and CDR3 sequences of SEQ ID Nos.: 93, 94, and 95, respectively, and a VL region comprising the CDR1, CDR2, and CDR3 sequences of SEQ ID Nos.: 128, XAS, wherein X is D or G, and 129, respectively, [613/613-08];
u) a VH region comprising the CDR1, CDR2, and CDR3 sequences of SEQ ID Nos.: 46, 119, and 120, respectively; and a VL region comprising CDR1, CDR2, and CDR3 sequences of SEQ ID Nos.: 49, AAS, and 50, respectively, [148/140];
v) a VH region comprising the CDR1, CDR2, and CDR3 sequences of SEQ ID Nos.: 123, 124, and 125, respectively; and a VL region comprising CDR1, CDR2, and CDR3 sequences of SEQ ID Nos.: 60, GAS, and 61, respectively [171/172/181];
w) a VH region comprising the CDR1, CDR2, and CDR3 sequences of SEQ ID Nos.: 121, 109, and 122, respectively; and a VL region comprising the CDR1, CDR2, and CDR3 sequences of SEQ ID Nos.: 112, AAS, and 113, respectively [726/187]; and
x) a VH region comprising the CDR1, CDR2, and CDR3 sequences of SEQ ID Nos.: 93, 126, and 127, respectively; and a VL region comprising the CDR1, CDR2, and CDR3 sequences of SEQ ID Nos.: 96, GAS, and 97, respectively [613/608-01/610-01/620-06].

16. The conjugate for use according to any one of the preceding claims, wherein the antibody comprises at least one binding region comprising a variable heavy chain (VH) region and a variable light chain (VL) region selected from the group consisting of:
a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
b) a VH region comprising the CDR1, CDR2, and CDR3 sequences of SEQ ID Nos.: 93, 94, and 95, respectively; and a VL region comprising the CDR1, CDR2, and CDR3 sequences of SEQ ID Nos.: 96, GAS, and 97, respectively, [613];
c) a VH region comprising the CDR1, CDR2, and CDR3 sequences of SEQ ID Nos.: 98, 99, and 100, respectively; and a VL region comprising the CDR1, CDR2, and CDR3 sequences of SEQ ID Nos.: 101, DAS, and 02, respectively, [613-08];
d) a VH region comprising the CDR1, CDR2, and CDR3 sequences of SEQ ID Nos.: 93, 94, and 95, respectively, and a VL region comprising the CDR1, CDR2, and CDR3 sequences of SEQ ID Nos.: 128, XAS, wherein X is D or G, and 129, respectively, [613/613-08]; and
e) a VH region comprising the CDR1, CDR2, and CDR3 sequences of SEQ ID Nos.: 93, 126, and 127, respectively; and a VL region comprising the CDR1, CDR2, and CDR3 sequences of SEQ ID Nos.: 96, GAS, and 97, respectively [613/608-01/610-01/620-06].

17. The conjugate for use according to any one of the preceding claims, wherein the antibody comprises at least one binding region comprising a variable heavy chain (VH) region and a variable light chain (VL) region selected from the group consisting of:
a) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148]; and
b) a VH region comprising the CDR1, CDR2, and CDR3 sequences of SEQ ID Nos.: 57, 58, and 59, respectively; and a VL region comprising the CDR1, CDR2, and CDR3 sequences of SEQ ID Nos.: 60, GAS, and 61, respectively, [171].

18. The conjugate for use according to any one of the preceding claims, wherein the antibody comprises at least one binding region comprising a variable heavy chain (VH) region and a variable light chain (VL) region selected from the group consisting of:
a) a VH region comprising the CDR1, CDR2, and CDR3 sequences of SEQ ID Nos.: 51, 52, and 53, respectively; and a VL region comprising the CDR1, CDR2, and CDR3 sequences of SEQ ID Nos.: 55, GAS, and 56, respectively [154];
b) a VH region comprising the CDR1, CDR2, and CDR3 sequences of SEQ ID Nos.: 72, 73, and 75, respectively; and a VL region comprising the CDR1, CDR2, and CDR3 sequences of SEQ ID Nos.: 76, ATS, and 77, respectively, [183]; and
c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733].

19. The conjugate for use according to any one of claims 1-15, wherein the antibody comprises a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively, [726].

20. The conjugate for use according to any one of claims 1-16, wherein the antibody comprises at least one binding region comprising a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107].

21. The conjugate for use according to any one of the preceding claims, wherein said at least one binding region comprises a VH region and a VL region selected from the group consisting of;
a) a VH region comprising an amino acid sequence having at least 90%, at least 95%, at least 97%, at least 99%, or 100% sequence identity to SEQ ID No: 1 and a VL region comprising an amino acid sequence having at least 90%, at least 95%, at least 97%, at least 99%, or 100% sequence identity to SEQ ID No: 2 [107];
b) a VH region comprising an amino acid sequence having at least 90%, at least 95%, at least 97%, at least 99%, or 100% sequence identity to SEQ ID No: 5 and a VL region comprising an amino acid sequence having at least 90%, at least 95%, at least 97%, at least 99%, or 100% sequence identity to SEQ ID No: 6 [148];
c) a VH region comprising an amino acid sequence having at least 90%, at least 95%, at least 97%, at least 99%, or 100% sequence identity to SEQ ID No: 34 and a VL region comprising an amino acid sequence having at least 90%, at least 95%, at least 97%, at least 99%, or 100% sequence identity to SEQ ID No: 35 [733];
d) a VH region comprising an amino acid sequence having at least 90%, at least 95%, at least 97%, at least 99%, or 100% sequence identity to SEQ ID No: 7 and a VL region comprising an amino acid sequence having at least 90%, at least 95%, at least 97%, at least 99%, or 100% sequence identity to SEQ ID No: 9 [154];
e) a VH region comprising an amino acid sequence having at least 90%, at least 95%, at least 97%, at least 99%, or 100% sequence identity to SEQ ID No: 10 and a VL region comprising an amino acid sequence having at least 90%, at least 95%, at least 97%, at least 99%, or 100% sequence identity to SEQ ID No: 11 [171];
f) a VH region comprising an amino acid sequence having at least 90%, at least 95%, at least 97%, at least 99%, or 100% sequence identity to SEQ ID No: 16 and a VL region comprising an amino acid sequence having at least 90%, at least 95%, at least 97%, at least 99%, or 100% sequence identity to SEQ ID No: 18 [183];
g) a VH region comprising an amino acid sequence having at least 90%, at least 95%, at least 97%, at least 99%, or 100% sequence identity to SEQ ID No: 25 and a VL region comprising an amino acid sequence having at least 90%, at least 95%, at least 97%, at least 99%, or 100% sequence identity to SEQ ID No: 26 [613];
h) a VH region comprising an amino acid sequence having at least 90%, at least 95%, at least 97%, at least 99%, or 100% sequence identity to SEQ ID No: 31 and a VL region comprising an amino acid sequence having at least 90%, at least 95%, at least 97%, at least 99%, or 100% sequence identity to SEQ ID No: 33 [726];
i) a VH region comprising an amino acid sequence having at least 90%, at least 95%, at least 97%, at least 99%, or 100% sequence identity to SEQ ID No: 3 and a VL region comprising an amino acid sequence having at least 90%, at least 95%, at least 97%, at least 99%, or 100% sequence identity to SEQ ID No: 4 [140];
j) a VH region comprising an amino acid sequence having at least 90%, at least 95%, at least 97%, at least 99%, or 100% sequence identity to SEQ ID No: 8 and a VL region comprising an amino acid sequence having at least 90%, at least 95%, at least 97%, at least 99%, or 100% sequence identity to SEQ ID No: 9 [154-M103L];
k) a VH region comprising an amino acid sequence having at least 90%, at least 95%, at least 97%, at least 99%, or 100% sequence identity to an amino acid sequence having at least 90%, at least 95%, at least 97%, at least 99%, or 100% sequence identity to SEQ ID No: 12 and a VL region comprising an amino acid sequence having at least 90%, at least 95%, at least 97%, at least 99%, or 100% sequence identity to SEQ ID No: 13 [172];
l) a VH region comprising an amino acid sequence having at least 90%, at least 95%, at least 97%, at least 99%, or 100% sequence identity to SEQ ID No: 14 and a VL region comprising an amino acid sequence having at least 90%, at least 95%, at least 97%, at least 99%, or 100% sequence identity to SEQ ID No: 15 [181];
m) a VH region comprising an amino acid sequence having at least 90%, at least 95%, at least 97%, at least 99%, or 100% sequence identity to SEQ ID No: 17 and a VL region comprising an amino acid sequence having at least 90%, at least 95%, at least 97%, at least 99%, or 100% sequence identity to SEQ ID No: 18 [183-N52Q];
n) a VH region comprising an amino acid sequence having at least 90%, at least 95%, at least 97%, at least 99%, or 100% sequence identity to SEQ ID No: 19 and a VL region comprising an amino acid sequence having at least 90%, at least 95%, at least 97%, at least 99%, or 100% sequence identity to SEQ ID No: 20 [187];
o) a VH region comprising an amino acid sequence having at least 90%, at least 95%, at least 97%, at least 99%, or 100% sequence identity to SEQ ID No: 21 and a VL region comprising an amino acid sequence having at least 90%, at least 95%, at least 97%, at least 99%, or 100% sequence identity to SEQ ID No: 22 [608-01];
p) a VH region comprising an amino acid sequence having at least 90%, at least 95%, at least 97%, at least 99%, or 100% sequence identity to SEQ ID No: 23 and a VL region comprising an amino acid sequence having at least 90%, at least 95%, at least 97%, at least 99%, or 100% sequence identity to SEQ ID No: 24 [610-01;
q) a VH region comprising an amino acid sequence having at least 90%, at least 95%, at least 97%, at least 99%, or 100% sequence identity to SEQ ID No: 27 and a VL region comprising an amino acid sequence having at least 90%, at least 95%, at least 97%, at least 99%, or 100% sequence identity to SEQ ID No: 28 [613-08];
r) a VH region comprising an amino acid sequence having at least 90%, at least 95%, at least 97%, at least 99%, or 100% sequence identity to SEQ ID No: 29 and a VL region comprising an amino acid sequence having at least 90%, at least 95%, at least 97%, at least 99%, or 100% sequence identity to SEQ ID No: 30 [620-06]; and
s) a VH region comprising an amino acid sequence having at least 90%, at least 95%, at least 97%, at least 99%, or 100% sequence identity to SEQ ID No: 32 and a VL region comprising an amino acid sequence having at least 90%, at least 95%, at least 97%, at least 99%, or 100% sequence identity to SEQ ID No: 33 [726-M101L].

22. The conjugate for use according to any one of the preceding claims, wherein said at least one binding region comprises a VH region and a VL region selected from the group consisting of;
a) a VH region comprising SEQ ID No: 1 and a VL region comprising SEQ ID No: 2 [107];
b) a VH region comprising SEQ ID No: 5 and a VL region comprising SEQ ID No: 6 [148];
c) a VH region comprising SEQ ID No: 34 and a VL region comprising SEQ ID No: 35 [733]
d) a VH region comprising SEQ ID No: 7 and a VL region comprising SEQ ID No: 9 [154];
e) a VH region comprising SEQ ID No: 10 and a VL region comprising SEQ ID No: 11 [171];
f) a VH region comprising SEQ ID No: 16 and a VL region comprising SEQ ID No: 18 [183];
g) a VH region comprising SEQ ID No: 25 and a VL region comprising SEQ ID No: 26 [613];
h) a VH region comprising SEQ ID No: 31 and a VL region comprising SEQ ID No: 33 [726];
i) a VH region comprising SEQ ID No: 3 and a VL region comprising SEQ ID No: 4 [140];
j) a VH region comprising SEQ ID No: 8 and a VL region comprising SEQ ID No: 9 [154-M103L];
k) a VH region comprising SEQ ID No: 12 and a VL region comprising SEQ ID No: 13 [172];
l) a VH region comprising SEQ ID No: 14 and a VL region comprising SEQ ID No: 15 [181];
m) a VH region comprising SEQ ID No: 17 and a VL region comprising SEQ ID No: 18 [183-N52Q];
n) a VH region comprising SEQ ID No: 19 and a VL region comprising SEQ ID No: 20 [187];
o) a VH region comprising SEQ ID No: 21 and a VL region comprising SEQ ID No: 22 [608-01];
p) a VH region comprising SEQ ID No: 23 and a VL region comprising SEQ ID No: 24 [610-01];
q) a VH region comprising SEQ ID No: 27 and a VL region comprising SEQ ID No: 28 [613-08];
r) a VH region comprising SEQ ID No: 29 and a VL region comprising SEQ ID No: 30 [620-06]; and
s) a VH region comprising SEQ ID No: 32 and a VL region comprising SEQ ID No: 33 [726-M101L].

23. The conjugate for use according to any one of the preceding claims, wherein said at least one binding region comprises a VH region and a VL region selected from the group consisting of:
a) a VH region comprising an amino acid sequence having at least 90%, at least 95%, at least 97%, at least 99%, or 100% sequence identity to SEQ ID No: 1 and a VL region comprising an amino acid sequence having at least 90%, at least 95%, at least 97%, at least 99%, or 100% sequence identity to SEQ ID No: 2 [107];
b) a VH region comprising an amino acid sequence having at least 90%, at least 95%, at least 97%, at least 99%, or 100% sequence identity to SEQ ID No: 25 and a VL region comprising an amino acid sequence having at least 90%, at least 95%, at least 97%, at least 99%, or 100% sequence identity to SEQ ID No: 26 [613];
c) a VH region comprising an amino acid sequence having at least 90%, at least 95%, at least 97%, at least 99%, or 100% sequence identity to SEQ ID No: 21 and a VL region comprising an amino acid sequence having at least 90%, at least 95%, at least 97%, at least 99%, or 100% sequence identity to SEQ ID No: 22 [608-01];
d) a VH region comprising an amino acid sequence having at least 90%, at least 95%, at least 97%, at least 99%, or 100% sequence identity to SEQ ID No: 23 and a VL region comprising an amino acid sequence having at least 90%, at least 95%, at least 97%, at least 99%, or 100% sequence identity to SEQ ID No: 24 [610-01];
e) a VH region comprising an amino acid sequence having at least 90%, at least 95%, at least 97%, at least 99%, or 100% sequence identity to SEQ ID No: 27 and a VL region comprising an amino acid sequence having at least 90%, at least 95%, at least 97%, at least 99%, or 100% sequence identity to SEQ ID No: 28 [613-08]; and
f) a VH region comprising an amino acid sequence having at least 90%, at least 95%, at least 97%, at least 99%, or 100% sequence identity to SEQ ID No: 29 and a VL region comprising an amino acid sequence having at least 90%, at least 95%, at least 97%, at least 99%, or 100% sequence identity to SEQ ID No: 30 [620-06].

24. The conjugate for use according to any one of the preceding claims, wherein said at least one binding region comprises a VH region and a VL region selected from the group consisting of:
a) a VH region comprising SEQ ID No: 1 and a VL region comprising SEQ ID No: 2 [107];
b) a VH region comprising SEQ ID No: 25 and a VL region comprising SEQ ID No: 26 [613];
c) a VH region comprising SEQ ID No: 21 and a VL region comprising SEQ ID No: 22 [608-01];
d) a VH region comprising SEQ ID No: 23 and a VL region comprising SEQ ID No: 24 [610-01];
e) a VH region comprising SEQ ID No: 27 and a VL region comprising SEQ ID No: 28 [613-08]; and
f) a VH region comprising SEQ ID No: 29 and a VL region comprising SEQ ID No: 30 [620-06].

25. The conjugate for use according to any one of the preceding claims, wherein said at least one binding region comprises a VH region and a VL region selected from the group consisting of:
c) a VH region comprising an amino acid sequence having at least 90%, at least 95%, at least 97%, at least 99%, or 100% sequence identity to SEQ ID No: 5 and a VL region comprising an amino acid sequence having at least 90%, at least 95%, at least 97%, at least 99%, or 100% sequence identity to SEQ ID No: 6 [148];
d) a VH region comprising an amino acid sequence having at least 90%, at least 95%, at least 97%, at least 99%, or 100% sequence identity to SEQ ID No: 10 and a VL region comprising an amino acid sequence having at least 90%, at least 95%, at least 97%, at least 99%, or 100% sequence identity to SEQ ID No: 11 [171].

26. The conjugate for use according to any one of the preceding claims, wherein said at least one binding region comprises a VH region and a VL region selected from the group consisting of:
c) a VH region comprising SEQ ID No: 5 and a VL region comprising SEQ ID No: 6 [148];
d) a VH region comprising SEQ ID No: 10 and a VL region comprising SEQ ID No: 11 [171].

27. The conjugate for use according to any one of the preceding claims, wherein said at least one binding region comprises a VH region and a VL region selected from the group consisting of:
d) a VH region comprising an amino acid sequence having at least 90%, at least 95%, at least 97%, at least 99%, or 100% sequence identity to SEQ ID No: 7 and a VL region comprising an amino acid sequence having at least 90%, at least 95%, at least 97%, at least 99%, or 100% sequence identity to SEQ ID No: 9 [154];
e) a VH region comprising an amino acid sequence having at least 90%, at least 95%, at least 97%, at least 99%, or 100% sequence identity to SEQ ID No: 16 and a VL region comprising an amino acid sequence having at least 90%, at least 95%, at least 97%, at least 99%, or 100% sequence identity to SEQ ID No: 18 [183];
f) a VH region comprising an amino acid sequence having at least 90%, at least 95%, at least 97%, at least 99%, or 100% sequence identity to SEQ ID No: 34 and a VL region comprising an amino acid sequence having at least 90%, at least 95%, at least 97%, at least 99%, or 100% sequence identity to SEQ ID No: 35 [733].

28. The conjugate for use according to any one of the preceding claims, wherein said at least one binding region comprises a VH region and a VL region selected from the group consisting of:
d) a VH region comprising SEQ ID No: 7 and a VL region comprising SEQ ID No: 9 [154];
e) a VH region comprising SEQ ID No: 16 and a VL region comprising SEQ ID No: 18 [183];
f) a VH region comprising SEQ ID No: 34 and a VL region comprising SEQ ID No: 35 [733].

29. The conjugate for use according to any one of the preceding claims, wherein said at least one binding region comprises a VH region comprising SEQ ID No: 31 and a VL region comprising SEQ ID No: 33 [726].

30. The conjugate for use according to any one of the preceding claims, wherein said at least one binding region comprises a VH region comprising SEQ ID No: 1 and a VL region comprising SEQ ID No: 2 [107].

31. The conjugate for use according to any one of the preceding claims, wherein said antibody comprises a heavy chain of an isotype selected from the group consisting of IgG1, IgG2, IgG3, and IgG4.

32. The conjugate for use according to any one of the preceding claims, wherein the isotype is IgG1, optionally allotype IgG1m(f).

33. The conjugate for use according to any one of the preceding claims, wherein the antibody is a full-length monoclonal antibody, such as a full-length monoclonal IgG1,κ antibody.

34. The conjugate for use according to any one of the preceding claims, wherein the antibody is a human antibody or a humanized antibody.

35. The conjugate for use according to any one of the preceding claims, wherein the antibody is enapotamab or a biosimilar thereof.

36. The conjugate for use according to any one of the preceding claims, wherein said cytotoxic agent is linked to said antibody with a cleavable linker, such as N-succinimydyl 4-(2-pyridyldithio)-pentanoate (SSP), maleimidocaproyl-valine-citrulline-p-aminobenzyloxycarbonyl (mc-vc-PAB) or AV-1 K-lock valine-citrulline.

37. The conjugate for use according to any one of claims 1-35, wherein said cytotoxic agent is linked to said antibody with a non-cleavable linker, such as succinimidyl-4(N-maleimidomethyl)cyclohexane-1-carboxylate (MCC) or maleimidocaproyl (MC).

38. The conjugate for use according to any one of the preceding claims, wherein said cytotoxic agent is selected from the group: DNA-targeting agents, e.g. DNA alkylators and cross-linkers, such as calicheamicin, duocarmycin, rachelmycin (CC-1065), pyrrolo[2,1-c][1,4] benzodiazepines (PBDs), and indolinobenzodiazepine (IGN); microtubule-targeting agents, such as duostatin, such as duostatin-3, auristatin, such as monomethylauristatin E (MMAE) and monomethylauristatin F (MMAF), dolastatin, maytansine, N(2′)-deacetyl-N(2′)-(3-marcapto-1-oxopropyl)-maytansine (DM1), and tubulysin; and nucleoside analogs; or an analogs, derivatives, or prodrugs thereof.

39. The conjugate for use according to any one of the preceding claims, wherein said conjugate has bystander kill capacity.

40. The conjugate for use according to any one of the preceding claims, wherein the cytotoxic agent is MMAE or a functional analog or derivative thereof.

41. The conjugate for use according to any one of the preceding claims, wherein the linker is mc-vc-PAB and the cytotoxic agent is MMAE.

42. The conjugate for use according to any one of claims 38-41, wherein MMAE is linked to the antibody via a mc-vc-PAB linker and the cytotoxic agent and the linker have the chemical structure; 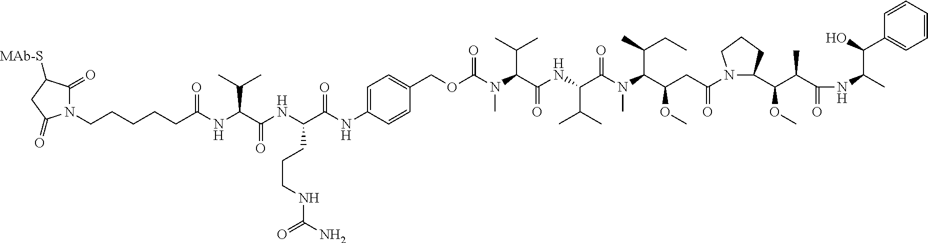
wherein MAb is the antibody.

43. The conjugate for use according to any one of claims 38-42, wherein MMAE is conjugated to the antibody via a cysteine thiol or a lysine.

44. The conjugate for use according to any one of the preceding claims, said conjugate having a Drug-to-Antibody Ratio (DAR) which is within the range of 1-8, such as 1-7, 1-6, 1-5, 1-4, 1-3, 1-2, 2-7, 2-6, 2-5, 2-4, 2-3, 3-8, 3-7, 3-6, 3-5, 3-4, 4-8, 4-7, 4-6, 4-5, 5-8, 5-7, 5-6, 6-8, 6-7, or 7-8, the DAR being the average number of cytotoxic agent molecules conjugated to each antibody molecule.

45. The conjugate for use according to any one of the preceding claims, the conjugate being enapotamab vedotin or a biosimilar thereof.

46. The conjugate for use according to any one of the preceding claims, wherein the antibody comprises at least one binding region comprising a variable heavy chain (VH) region and a variable light chain (VL) region selected from the group consisting of:
f) a VH region comprising an amino acid sequence having at least 90%, at least 95%, at least 97%, at least 99%, or 100% sequence identity to SEQ ID No: 139 and a VL region comprising an amino acid sequence having at least 90%, at least 95%, at least 97%, at least 99%, or 100% sequence identity to SEQ ID No: 140;
g) a VH region comprising an amino acid sequence having at least 90%, at least 95%, at least 97%, at least 99%, or 100% sequence identity to SEQ ID No: 141 and a VL region comprising an amino acid sequence having at least 90%, at least 95%, at least 97%, at least 99%, or 100% sequence identity to SEQ ID No: 142;
h) a VH region comprising an amino acid sequence having at least 90%, at least 95%, at least 97%, at least 99%, or 100% sequence identity to SEQ ID No: 141 and a VL region comprising an amino acid sequence having at least 90%, at least 95%, at least 97%, at least 99%, or 100% sequence identity to SEQ ID No: 143;
a VH region comprising an amino acid sequence having at least 90%, at least 95%, at least 97%, at least 99%, or 100% sequence identity to SEQ ID No: 144 and a VL region comprising an amino acid sequence having at least 90%, at least 95%, at least 97%, at least 99%, or 100% sequence identity to SEQ ID No: 142; and
j) a VH region comprising an amino acid sequence having at least 90%, at least 95%, at least 97%, at least 99%, or 100% sequence identity to SEQ ID No: 144 and a VL region comprising an amino acid sequence having at least 90%, at least 95%, at least 97%, at least 99%, or 100% sequence identity to SEQ ID No: 143.

47. The conjugate for use according to any one of the preceding claims, wherein the antibody is conjugated to a pyrrolobenzodiazepine, such as a pyrrolobenzodiazepine (PBD) dimer.

48. The conjugate for use according to any one of the preceding claims, wherein the conjugate is ADCT-601 (ADC Therapeutics).

49. The conjugate for use according to any one of the preceding claims, wherein the antibody comprises at least one binding region comprising a variable heavy chain (VH) region and a variable light chain (VL) region, the VH region and the VL region comprising amino acids sequences encoded by the nucleic acid sequences set forth in:
a) SEQ ID NOs: 145 and 146, respectively;
b) SEQ ID NOs: 145 and 147, respectively;
c) SEQ ID NOs: 145 and 148, respectively;
d) SEQ ID NOs: 145 and 149, respectively;
e) SEQ ID NOs: 150 and 146, respectively;
f) SEQ ID NOs: 150 and 147, respectively;
g) SEQ ID NOs: 150 and 148, respectively;
h) SEQ ID NOs: 150 and 149, respectively;
i) SEQ ID NOs: 151 and 146, respectively;
j) SEQ ID NOs: 151 and 147, respectively;
k) SEQ ID NOs: 151 and 148, respectively; and
l) SEQ ID NOs: 151 and 149, respectively.

50. The conjugate for use according to any one of the preceding claims, wherein the conjugate is CAB-AXL-ADC/BA3011 (BioAtla).

51. The conjugate for use according to any one of claims 2-50, wherein the inhibitor of PD-1 and/or PD-L1 is an inhibitor of the interaction between PD-1 and its ligand; e.g. PD-L1.

52. The conjugate for use according to any one of claims 2-51, wherein the the inhibitor of PD-1 and/or PD-L1 is or comprises an antibody, such as an antagonistic antibody, or antigen-binding fragment thereof.

53. The conjugate for use according to any one of claims 2-52, wherein the inhibitor of PD-1 and/or PD-L1 is selected from the group consisting of pembrolizumab (Merck & Co), CBT-501 (genolimzumab; Genor Bio/CBT Pharma), nivolumab (BMS), REGN2810 (Cemiplimab; Regeneron), BGB-A317 (Tislelizumab; BeiGene/Celgene), Amp-514 (MEDI0680) (Amplimmune), TSR-042 (Dostarlimab; Tesaro/AnaptysBio), JNJ-63723283/JNJ-3283 (Johnson & Johnson), PF-06801591 (Pfizer), JS-001 (Tripolibamab/Toripalimab; Shanghai Junshi Bio), SHR-1210/INCSHR-1210 (Camrelizumab; Incyte corp), PDR001 (Spartalizumab; Novartis), BCD-100 (BioCad), AGEN2034 (Agenus), IBI-308 (Sintilimab; Innovent Biologics), B1-754091 (Boehringer Ingelheim), GLS-010 (WuXi/Arcus), LZM-009 (Livzon MabPharm), AMG-404 (Amgen), CX-188 (CytomX), ABBV-181 (Abbvie), BAT-1306 (BioThera), JTX-4014 (Jounce Therapeutics), AK-103 (Akeso Bio), AK-105 (Akeso Bio), MGA-012 (Macrogenics/Incyte), Sym-021 (Symphogen), AB122 (Arcus Biosciences/Strata Oncology), CS1003 (C-Stone), 609A (Sunshine Guojian), hAB21 (Suzhou Stainwei Biotech), SCT-110A (Sinocelltech), HLX-10 (Shanghai Henlius Biotech), HX-008 (Taizhou Hanzhong Biomedical).

54. The conjugate for use according to any one of claims 2-53, wherein the inhibitor of PD-1 is pembrolizumab (Merck & Co).

55. The conjugate for use according to any one of claims 2-52, wherein the the inhibitor of PD-1 and/or PD-L1 comprises an antibody, or antigen-binding fragment thereof, capable of binding to PD-L1.

56. The conjugate for use according to any one of claims 2-51 and 55, wherein the inhibitor of PD-1 and/or PD-L1 is selected from the group consisting of RG7446/MPDL-3280A (atezolizumab; Roche), MSB-0010718C (avelumab; Merck Serono/Pfizer) and MEDI-4736 (durvalumab; AstraZeneca), KN-035 (envafolimab; 3DMed/Alphamab Co.), CX-072 (CytomX), LY-3300054 (Eli Lilly), STI-A1014 (Sorrento/Lees Pharm), A167 (Harbour BioMed/Kelun biotech), BGB-A333 (BeiGene), MSB0011359C (M-7824) (Bintrafusp alfa; Merck KGaA), FAZ053 (Novartis), BCD-135 (Biocad), HLX-20 (Shanghai Henlius Bio), AK-106 (Akeso), KL-A167 (Kelun), SHR-1316 (Atridia), CA-170 (Aurigene/Curis), LP-002 (Lepu Pharmaceuticals), MSB2311 (MabSpace), CK-301 (Cosibelimab; Checkpoint Therapeutics and TG Ther.), CS1001/WBP-3155 (C-Stone Pharmaceuticals), IMC-001 (ImmuneOncia/Sorrento), WBP3155 (C-Stone Pharm), ZKAB001 (Sorrento/Lee's Pharma), JS-003 (Shanghai Junshi Biosciences), CBT-502 (CBT Pharmaceuticals), GS-4224 (Gilead), TG-1501 (TG Therapeutics), CBT-502 (CBT Pharmaceuticals).

57. The conjugate for use according to any one of the preceding claims, wherein the conjugate has antitumor activity or is able to induce tumor regression in NSCLC and/or melanoma xenograft models, such as a BLM melanoma xenograft or a LCLC-103H xenograft model.

58. The conjugate for use according to claim 57, wherein the NSCLC and/or melanoma xenograft model is resistant to treatment with one or more inhibitors of PD-1 and/or PD-L1, such as one ore more inhibitors of PD-1 and/or PD-L1 as defined in any one of claims 51-56.

59. The conjugate for use according to claim 57 or 58, wherein the BLM melanoma xenograft model is generated as described in Example 3 herein or is generated essentially as described in Example 3 herein.

60. The conjugate for use according to claim 57 or 58, wherein the NSCLC xenograft model is generated as described in Example 4 herein or is generated essentially as described in Example 4 herein.

61. The conjugate for use according to any one of the preceding claims, wherein immunogenic cell death and/or tumor-associated inflammation is characterised by
d) increased release of ATP from cells of said tumor,
e) secretion of high-mobility group box 1 (HMGB1) from cells of said tumor, and/or
f) cell surface expression of Calreticulin on cells of said tumor.

62. The conjugate for use according to any one of the preceding claims, wherein the ability of said antibody to induce immunogenic cell death and/or tumor-associated inflammation is determined by measuring
d) release of ATP,
e) secretion of high-mobility group box 1 (HMGB1), and/or
f) cell surface expression of Calreticulin,
in a cancer cell line, such as in a human Non-Small Cell Lung Cancer cell line or a human breast cancer cell line.

63. The conjugate for use according to claim 61, wherein release of ATP, secretion of HGMB1 and/or cell surface expression of Calreticulin is determined as described in Example 6 herein.

64. The conjugate for use according to claim 61 or 62, wherein the human Non-Small Cell Lung Cancer cell line is LCLC-103H and/or wherein the human breast cancer cell line is MDA-MB-231.

65. The conjugate for use according to any one of claims 61-64, wherein release of ATP is determined in a process comprising
i) Establishing a culture said cancer cell line in 24 well plates and culturing the cells to for 3-4 hours at 37° C.,
ii) Adding said conjugate to the cells in an amount corresponding to 2 μg/ml, and incubating the cells for 48 hours at 37° C.,
iii) Washing the cells in phosphate buffered saline (PBS)
iv) Incubating the cells in 100% PBS or 70% PBS,
v) Harvesting the cells and separating cells and supernatant by centrifugation at 1000 rpm for 2 minutes at room temperature, and
vi) Measuring ATP in the supernatant.

66. The conjugate for use according to any one of claims 61-64, wherein secretion of HMGB1 is determined in a process comprising:
i) Establishing a culture said cancer cell line in 24 well plates and culturing the cells to for 3-4 hours at 37° C.,
ii) Adding said conjugate to the cells and incubating the cells for 48 hours at 37° C.,
iii) Separating cells and supernatant from the culture by centrifugation at 1000 rpm for 3 minutes at room temperature, and
iv) Measuring HMGB1 in the supernatant, such as by enzyme-linked immunosorbent assay (ELISA).

67. The conjugate for use according to any one of claims 61-64, wherein cell surface expression of Calreticulin is determined by a process comprising:
i) Establishing a culture of said cancer cell line, grown to 70-80% confluency and incubating the cancer cell line with the conjugate in amounts corresponding of 2 μg/ml for 48 hours at 37° C.,
ii) Collecting cells from the culture and washing the cells in FACS buffer (phosphate buffered saline (PBS)/0.55 w/w bovise serum albumin (BSA)/0.02% w/w azide),
iii) Incubating the cells with phycoerythrin (PE)-conjugated mouse anti-human calreticulin antibody for 30 minutes at 4° C. in darkness,
iv) Washing the cells in FACS buffer and analyzing the cells by flow cytometry.

68. The conjugate for use according to any of the preceding claims, wherein the subject is a human.

69. The conjugate for use according to any of the preceding claims, wherein the conjugate is administered to said subject in therapeutically effective amounts and frequencies; such as
In at least one cycle comprising administration once every three weeks, such as on day 1 of a cycle of 21 days; or
in at least one cycle comprising administration once a week for three consecutive weeks followed by a one-week resting period without any administration of ADC so that each cycle time is 28 days including the resting period, such as on days 1, 8 and 15 in the cycle of 28 days.

70. The conjugate for use according to claim 69, wherein the dose of the conjugate in said cycle of 21 days is between 0.6 mg/kg and 4.0 mg/kg of the subject's body weight, such as between 0.6 mg/kg and 3.2 mg/kg of the subject's body weight, such as at a dose of about 0.6 mg/kg or at a dose of about 0.8 mg/kg or at a dose of about 1.0 mg/kg or at a dose of about 1.2 mg/kg or at a dose of about 1.4 mg/kg or at a dose of about 1.6 mg/kg or at a dose of about 1.8 mg/kg or at a dose of about 2.0 mg/kg or at a dose of about 2.2 mg/kg or at a dose of about 2.4 mg/kg or at a dose of about 2.6 mg/kg or at a dose of about 2.8 mg/kg or at a dose of about 3.0 mg/kg or at a dose of about 3.2 mg/kg.

71. The conjugate for use according to claim 69, wherein the dose of the conjugate in said cycle of 28 days is between 0.45 mg/kg and 2.0 mg/kg of the subject's body weight, such as at a dose of 0.45 mg/kg or at a dose of 0.5 mg/kg or at a dose of 0.6 mg/kg or at a dose of 0.7 mg/kg or at a dose of 0.8 mg/kg or at a dose of 0.9 mg/kg or at a dose of 1.0 mg/kg or at a dose of 1.1 mg/kg or at a dose of 1.2 mg/kg or at a dose of 1.3 mg/kg or at a dose of 1.4 mg/kg or at a dose of 1.5 mg/kg or at a dose of 1.6 mg/kg or at a dose of 1.7 mg/kg or at a dose of 1.8 mg/kg or at a dose of 1.9 mg/kg or at a dose of 2.0 mg/kg.

72. The conjugate for use according to any one of claims 69-71, wherein the number of cycles of 21 days or the number of cycles of 28 days is between 2 and 48, such as between 2 and 36, such as between 2 and 24, such as between 2 and 15, such as between 2 and 12, such as 2 cycles, 3 cycles, 4 cycles, 5 cycles, 6 cycles, 7 cycles, 8 cycles, 9 cycles, 10 cycles, 11 cycles or 12 cycles.

73. The conjugate for use according to any one of the preceding claims, wherein the conjugate is administered for at least four treatment cycles of 28 days, wherein the antibody or ADC in each treatment cycle is administered once a week at a dose of 0.45 mg/kg body weight, such as at a dose of 0.6 mg/kg body weight, 0.8 mg/kg body weight, 1.0 mg/kg body weight, 1.2 mg/kg body weight, 1.4 mg/kg body weight, 1.6 mg/kg body weight, 1.8 mg/kg body weight, or such as 2.0 mg/kg body weight for three consecutive weeks followed by a resting week without any administration of the antibody or ADC.

74. The conjugate for use according to any one of the preceding claims, wherein the conjugate is administered to the subject at a dose of about 2.0-about 2.4 mg/kg body weight once every three weeks or by weekly dosing of about 0.6-about 1.4 mg/kg body weight for three weeks, optionally followed by one treatment-free week.

75. The conjugate for use according to any one of the preceding claims, wherein the conjugate is administered to the subject at a dose of about 2.2 mg/kg body weight once every three weeks or by weekly dosing of about 1.0 mg/kg body weight for three weeks, optionally followed by one treatment-free week.

76. The conjugate for use according to any one of the preceding claims, wherein treatment is continued at least until said subject has experienced progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the first dose of the conjugate.

77. The conjugate for use according to any one of the preceding claims, wherein treatment is continued until disease progression.

78. The conjugate for use according to any one of the preceding claims, wherein the conjugate is administered to the subject at a dose of about 1.8-about 2.6 mg/kg body weight once every three weeks or by weekly dosing of about 0.8-about 1.2 mg/kg body weight for three weeks, optionally followed by one treatment-free week.

79. The conjugate for use according to any one of the preceding claims, wherein said conjugate is administered by intravenous injection or infusion.

80. The conjugate for use according to any one of the preceding claims, wherein the cancer said cancer is a solid tumor, such as a metastasic, solid tumor, such as a metastasic, locally advanced tumor.

81. The conjugate for use according to any one of the preceding claims, wherein the cancer is selected from the group consisting of colorectal cancer, such as colorectal carcinoma and colorectal adenocarcinoma; bladder cancer, bone cancer such as chondrosarcoma; breast cancer such as triple-negative breast cancer; cancers of the central nervous system such as glioblastoma, astrocytoma, neuroblastoma; cervical cancer, connective tissue cancer, endometrium cancer, fibroblast cancer, gastric cancer such as gastric carcinoma; head and neck cancer, kidney cancer, liver cancer such as hepatocellular carcinoma; lung cancer such as NSCLC and lung squamous cell carcinoma; muscle cancer, neural tissue cancer, ovarian cancer, pancreatic cancer such as pancreatic ductal carcinoma and pancreatic adenocarcinoma; skin cancer such as malignant melanoma; soft tissue sarcoma and mesothelioma.

82. The conjugate for use according to any one of the preceding claims, wherein the cancer is selected from the group consisting of non-small cell lung cancer (NSCLC), melanoma, Sarcoma, cervical cancer, endometrial cancer and ovarian cancer, pancreatic cancer, bladder head and neck.

83. The conjugate for use according to any one of the preceding claims, wherein the cancer is non-small cell lung cancer (NSCLC).

84. The conjugate for use according to any one of the preceding claims, wherein the subject has received prior treatment with a PD-1 pathway inhibitor; e.g. an inhibitor of PD-1 and/or PD-L1 as defined in any one of claims 51-56.

85. The conjugate for use according to any one of the preceding claims, wherein said cancer has previously been treated with a PD-1 pathway inhibitor, such as an inhibitor of PD-1 and/or PD-L1 as defined in any one of claims 51-56.

86. The conjugate for use according to any one of the preceding claims, wherein the cancer and/or the subject is resistant to, has failed to respond to has relapsed from prior treatment with a PD-1 pathway inhibitor, such as an inhibitor of PD-1 and/or PD-L1 as defined in any one of claims 51-56.

87. The conjugate for use according to any one of claims 84-86, wherein treatment with said PD-1 pathway inhibitor was the last treatment prior to treatment with said conjugate and inhibitor of PD-1 and/or PD-L1 as defined in any one of the preceding claims.

88. The conjugate for use according to any one of the preceding claims, wherein the cancer and/or the subject has primary (de novo) or acquired resistance to a PD-1 pathway inhibitor, such as an inhibitor of PD-1 and/or PD-L1 as defined in any one of claims 51-56.

89. The conjugate for use according to any one of claims 84-88, wherein resistance to, failure to respond to and/or relapse from treatment with a PD-1 pathway inhibitor has been determined according to the Response Evaluation Criteria In Solid Tumors; version 1.1 (RECIST Criteria v1.1).

90. The conjugate for use according to any one of claims 84-89, wherein said subject has Stable Disease (SD) or Progressive Disease (PD); such as determined according to the RECIST Criteria v1.1.

91. An inhibitor of PD-1 and/or PD-L1 for use in treating cancer in a subject, in combination with a conjugate of a cytotoxic agent and an antibody or antigen-binding fragment thereof capable of binding to human Axl; e.g. human Axl having the sequence set forth in SEQ ID NO: 1.

92. The inhibitor of PD-1 and/or PD-L1 for use according to claim 91, wherein the PD-1 pathway inhibitor is as defined in any one of claims 51-56.

93. The inhibitor of PD-1 and/or PD-L1 for use according to claim 91 or 92, wherein the antibody is as defined in any one of claims 1-35, 46, 47 and 49.

94. The inhibitor of PD-1 and/or PD-L1 for use according to any one of claims 91-93, wherein the cytotoxic agent is as defined in any one of claims 36-44.

95. The inhibitor of PD-1 and/or PD-L1 for use according to any one claims 91-94 wherein the cancer is as defined in any one of claims 80-90.

96. The inhibitor of PD-1 and/or PD-L1 for use according to any one of claims 91-9586, wherein the subject is as defined in any one of claims 86, 88 and 89.

97. The inhibitor of PD-1 and/or PD-L1 for use according to any one of claims 91-96, wherein the PD-1 pathway inhibitor and/or the conjugate is/are administered to the subject as is defined in any one of claims 69-79.

98. A method of potentiating the therapeutic efficacy of PD-1 and/or PD-L1 inhibition, comprising administering to a subject in need thereof a conjugate of a cytotoxic agent and an antibody capable of binding to human Axl (e.g. human Axl having the sequence set forth in SEQ ID NO: 1), thereby inducing immunogenic cell death and/or tumor-associated inflammation; e.g. tumor-associated inflammation associated with immunogenic cell death.

99. The method according to claim 98, wherein the conjugate is administered in combination with an inhibitor of programmed cell death-1 (PD-1) and/or programmed death-ligand 1 (PD-L1).

100. A method of treating cancer comprising administering to a subject in need thereof
an inhibitor of PD-1 and/or PD-L1; and
a conjugate of a cytotoxic agent and an antibody or antigen-binding fragment thereof capable of binding to human Axl; e.g. human Axl having the sequence set forth in SEQ ID NO: 130.

101. The method according to claim 99 or 100, wherein the inhibitor of PD-1 and/or PD-L1 is as defined in any one of claims 51-56.

102. The method according to claim 99 or 101, wherein the antibody is as defined in any one of claims 1-35, 46, 47 and 49.

103. The method according to any one of claims 100-102, wherein the cytotoxic agent is as defined in any one of claims 36-45.

104. The method according to any one claims 100-103, wherein the cancer is as defined in any one of claims 80-90.

105. The method according to any one of claims 100-104, wherein the subject is as defined in any one of claims 86, 88 and 89.

106. The method according to any one of claims 100-105, wherein the inhibitor of PD-1 and/or PD-L1 and/or the conjugate is/are administered to the subject is as defined in any one of claims 69-79.

107. The method according to any one of claims 100-106, wherein immunogenic cell death and/or tumor-associated inflammation is characterized as defined in claim 61 and/or is determined as set forth in any one of claims 62-67.

108. A pharmaceutical composition or formulation comprising a conjugate of an antibody or antigen-binding fragment thereof capable of binding to human Axl; e.g. human Axl having the sequence set forth in SEQ ID NO: 130, and an inhibitor of PD-1 and/or PD-L1.

109. The pharmaceutical composition according to claim 96, wherein the inhibitor of PD-1 and/or PD-L1 is as defined in any one of claims 51-56.

110. The pharmaceutical composition according to claim 108 or 109, wherein the antibody is as defined in any one of claims 1-35, 46, 47 and 49.

111. The pharmaceutical composition according to any one of claims 108-110, wherein the cytotoxic agent is as defined in any one of claims 36-45.

112. A kit of parts comprising a conjugate of an antibody or antigen-binding fragment thereof capable of binding to human Axl; e.g. human Axl having the sequence set forth in SEQ ID NO: 130, and an inhibitor of PD-1 and/or PD-L1.

113. The kit according to claim 112, wherein the inhibitor of PD-1 and/or PD-L1 is as defined in any one of claims 51-56.

114. The kit according to claim 112 or 113, wherein the antibody is as defined in any one of claims 1-35, 46, 47 and 49.

115. The kit according to any one of claims 112-114, wherein the cytotoxic agent is as defined in any one of claims 36-45.


SEQUENCE LISTING (154 sequences)

SEQ ID NO: 1 - protein, 116 aa, homo sapiens
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 2 - protein, 108 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 3 - protein, 120 aa, homo sapiens
EVQLLESGGGLVQPGGSLRLSCAASGFTFSSYAMTWVRQAPGKGLEWVSAISISGASTFYADSVKGRFTISRDNSKNTLSLQMNSLRAEDTAVYFCRGYSGYVYDAFDIWGQGTMVTVSS

SEQ ID NO: 4 - protein, 107 aa, homo sapiens
DIQMTQSPSSLSASVGDRVTITCRASQGISNWLAWYQQKPEKAPKSLIYAASSLQSGVPSRFSGSGSGTDFTLTISSLQPEDFATYYCQQYNSYPLTFGGGTKVEIK

SEQ ID NO: 5 - protein, 120 aa, homo sapiens
EVQLLESGGGLVQPGGSLRLSCAASGFTFSSYAMTWVRQAPGKGLEWVSAISISGGSTFYADSVKGRFTISRDNSKNTLYLQMNSLRAEDTAVYYCRGYSGYVYDAFDFWGQGTMVTVSS

SEQ ID NO: 6 - protein, 107 aa, homo sapiens; identical to SEQ ID NO: 4

SEQ ID NO: 7 - protein, 121 aa, homo sapiens
EVQLLDSGGGLVQPGGSLRLSCAASGFTFSSYAMSWVRQAPGKGLEWVSAISIGGGNAYYADSVKGRFTISRDNSKNTLYLQMNSLRAADTAVYYCAKPGFIMVRGPLDYWGQGALVTVSS

SEQ ID NO: 8 - protein, 121 aa, homo sapiens
EVQLLDSGGGLVQPGGSLRLSCAASGFTFSSYAMSWVRQAPGKGLEWVSAISIGGGNAYYADSVKGRFTISRDNSKNTLYLQMNSLRAADTAVYYCAKPGFILVRGPLDYWGQGALVTVSS

SEQ ID NO: 9 - protein, 108 aa, homo sapiens
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPYTFGQGTKLEIK

SEQ ID NO: 10 - protein, 125 aa, homo sapiens
EVQLLESGGGLVQPGGSLRLSCAASGFTFSSYAMSWVRQAPGKGLEWVSDISVSGGSTYYADSVKGRFTISRDNSKNTLYLQMNSLRAEDTAVYYCAKEGYIWFGESLSYAFDIWGQGTMVTVSS

SEQ ID NO: 11 - protein, 107 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGRSFTFGPGTKVDIK

SEQ ID NO: 12 - protein, 125 aa, homo sapiens
EVQLLESGGGLVQPGGSLRLSCAASGFTFSNYAMSWVRQAPGKGLEWVSDISVSGGSTYYADSVKGRFTISRDNSKNTLYLQMNSLRAEDTAVYYCAKEGYIWFGESLSYAFDIWGQGTMVTVSS

SEQ ID NO: 13 - protein, 107 aa, homo sapiens; identical to SEQ ID NO: 11

SEQ ID NO: 14 - protein, 125 aa, homo sapiens
EVQLLESGGGLVQPGGSLRLSCAASGFTFSSYAMSWVRQAPGKGLEWVSDISVSGGSTYYADSVKGRFTISRDNSKNTLYLHMNSLRAEDTAVYYCAKEGYIWFGESLSYAFDIWGQGTMVTVSS

SEQ ID NO: 15 - protein, 107 aa, homo sapiens; identical to SEQ ID NO: 11

SEQ ID NO: 16 - protein, 117 aa, homo sapiens
QVQLQQWGAGLLKPSETLSLTCAVYGGSFSGYYWSWIRQPPGKGLEWIGEINQSGSTNYNPSLKSRVTISVDTSKNQFSLKLSSVTAADTSVYYCASGNWDHFFDYWGQGTLVTVSS

SEQ ID NO: 17 - protein, 117 aa, homo sapiens
QVQLQQWGAGLLKPSETLSLTCAVYGGSFSGYYWSWIRQPPGKGLEWIGEIQQSGSTNYNPSLKSRVTISVDTSKNQFSLKLSSVTAADTSVYYCASGNWDHFFDYWGQGTLVTVSS

SEQ ID NO: 18 - protein, 107 aa, homo sapiens
DIQMTQSPSSVSASVGDRVTITCRASQGISSWLAWYQHKPGKAPKLLIYATSSLQSGVTSRFSGSGSGTDFTLTISSLQPEDFATYYCQQAKSFPWTFGQGTKVEIK

SEQ ID NO: 19 - protein, 123 aa, homo sapiens
QVPLQQWGAGLLKPSETLSLTCAVYGGSFSGYHWSWIRQPPGKGLEWIGEISHSGRTNYNPSLKSRVTISIDTSKNQFSLKLSSVTAADTAVYYCASFITMIRGTIITHFDYWGQGTLVTVSS

SEQ ID NO: 20 - protein, 107 aa, homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYHSYPYTFGQGTKLEIK

SEQ ID NO: 21 - protein, 124 aa, homo sapiens
QVQLVQSGAEVKKPGSSVKVSCKASGGTFSSYAISWVRQAPGQGLEWMGRIIPIFGIANYVQKFQGRVTITADKSTSTAYMELSSLRAEDTAVYYCARRGDYYGSGSPDVFDIWGQGTMVTVSS

SEQ ID NO: 22 - protein, 107 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSYTFGQGTKLEIK

SEQ ID NO: 23 - protein, 124 aa, homo sapiens
QVQLVQSGAEVKKPGSSVKVSCKASGGTFSSYAISWVRQAPGQGLEWMGRIIPIFGIANYVQKFQGRVTITADKSTSTAYMELSSLRAEDTAVYYCARRGNYYGSGSPDVFDIWGQGTMVTVSS

SEQ ID NO: 24 - protein, 107 aa, homo sapiens; identical to SEQ ID NO: 22

SEQ ID NO: 25 - protein, 124 aa, homo sapiens
QVQLVQSGAEVKKPGSSVKVSCKASGGTFSSYAINWMRQAPGQGLEWMGRIIPIFGIVNYAQKFQGRVTLTADKSTSTAYMELSSLRSEDTAVYYCARRGNYYGSGSPDVFDIWGQGTMVTVSS

SEQ ID NO: 26 - protein, 107 aa, homo sapiens; identical to SEQ ID NO: 22

SEQ ID NO: 27 - protein, 124 aa, homo sapiens; identical to SEQ ID NO: 25

SEQ ID NO: 28 - protein, 106 aa, homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWLTFGGGTKVEIK

SEQ ID NO: 29 - protein, 124 aa, homo sapiens
QVQLVQSGAEVKKPGSSVKVSCKASGGTFSSYAISWVRQAPGQGLEWMGRIIPIFGIANYAQKFQGRVTITADKSTSTAYMELSSLRSEDTAVYYCARRGNYYGSGSPDVFDIWGQGTMVTVSS

SEQ ID NO: 30 - protein, 107 aa, homo sapiens; identical to SEQ ID NO: 22

SEQ ID NO: 31 - protein, 123 aa, homo sapiens
QVQLQQWGAGLLKPSETLSLTCAIDGGSFSGYYWSWIRQPPGKGLEWIGEISHSGRTNYNPSLKSRVTISIDTSKNQFSLKLSSVAAADTAVYYCARFITMIRGAIITHFDYWGQGALVTVSS

SEQ ID NO: 32 - protein, 123 aa, homo sapiens
QVQLQQWGAGLLKPSETLSLTCAIDGGSFSGYYWSWIRQPPGKGLEWIGEISHSGRTNYNPSLKSRVTISIDTSKNQFSLKLSSVAAADTAVYYCARFITLIRGAIITHFDYWGQGALVTVSS

SEQ ID NO: 33 - protein, 107 aa, homo sapiens; identical to SEQ ID NO: 20

SEQ ID NO: 34 - protein, 120 aa, homo sapiens
QVQLVESGGGVVQPGRSLRLSCAASGFSFSTYAMHWVRQAPGKGLEWVAVISYDGDNKYSADSVKGRFTISRDNSKNTLYLQMNSLRAEDTAVYYCARGRKLGIDAFDIWGQGTMVTVSS

SEQ ID NO: 35 - protein, 107 aa, homo sapiens
AIQLTQSPSSLSASVGDRVTITCRASQGISSALAWYQQKPGKAPKLLIYDASSLESGVPSRFSGSGSGTDFTLTISGLQPEDFATYYCQQFNSYPFTFGPGTKVDIK

SEQ ID NO: 36 - protein, 8 aa, homo sapiens
GFTFSSYA

SEQ ID NO: 37 - protein, 8 aa, homo sapiens
TSGSGAST

SEQ ID NO: 38 - protein, 9 aa, homo sapiens
AKIWIAFDI

SEQ ID NO: 39 - protein, 7 aa, homo sapiens
QSVSSSY

SEQ ID NO: 40 - protein, 9 aa, homo sapiens
QQYGSSPYT

SEQ ID NO: 41 - protein, 8 aa, homo sapiens
GFTFSSYA

SEQ ID NO: 42 - protein, 8 aa, homo sapiens
ISISGAST

SEQ ID NO: 43 - protein, 13 aa, homo sapiens
RGYSGYVYDAFDI

SEQ ID NO: 44 - protein, 6 aa, homo sapiens
QGISNW

SEQ ID NO: 45 - protein, 9 aa, homo sapiens
QQYNSYPLT

SEQ ID NO: 46 - protein, 8 aa, homo sapiens
GFTFSSYA

SEQ ID NO: 47 - protein, 8 aa, homo sapiens
ISISGGST

SEQ ID NO: 48 - protein, 13 aa, homo sapiens
RGYSGYVYDAFDF

SEQ ID NO: 49 - protein, 6 aa, homo sapiens
QGISNW

SEQ ID NO: 50 - protein, 9 aa, homo sapiens
QQYNSYPLT

SEQ ID NO: 51 - protein, 8 aa, homo sapiens
GFTFSSYA

SEQ ID NO: 52 - protein, 8 aa, homo sapiens
ISIGGGNA

SEQ ID NO: 53 - protein, 14 aa, homo sapiens
AKPGFIMVRGPLDY

SEQ ID NO: 54 - protein, 14 aa, homo sapiens
AKPGFILVRGPLDY

SEQ ID NO: 55 - protein, 7 aa, homo sapiens
QSVSNSY

SEQ ID NO: 56 - protein, 9 aa, homo sapiens
QQYGSSPYT

SEQ ID NO: 57 - protein, 8 aa, homo sapiens
GFTFSSYA

SEQ ID NO: 58 - protein, 8 aa, homo sapiens
ISVSGGST

SEQ ID NO: 59 - protein, 18 aa, homo sapiens
AKEGYIWFGESLSYAFDI

SEQ ID NO: 60 - protein, 7 aa, homo sapiens
QSVSSSY

SEQ ID NO: 61 - protein, 8 aa, homo sapiens
QQYGRSFT

SEQ ID NO: 62 - protein, 8 aa, homo sapiens
GFTFSNYA

SEQ ID NO: 63 - protein, 8 aa, homo sapiens
ISVSGGST

SEQ ID NO: 64 - protein, 18 aa, homo sapiens
AKEGYIWFGESLSYAFDI

SEQ ID NO: 65 - protein, 7 aa, homo sapiens
QSVSSSY

SEQ ID NO: 66 - protein, 8 aa, homo sapiens
QQYGRSFT

SEQ ID NO: 67 - protein, 8 aa, homo sapiens
GFTFSSYA

SEQ ID NO: 68 - protein, 8 aa, homo sapiens
ISVSGGST

SEQ ID NO: 69 - protein, 18 aa, homo sapiens
AKEGYIWFGESLSYAFDI

SEQ ID NO: 70 - protein, 7 aa, homo sapiens
QSVSSSY

SEQ ID NO: 71 - protein, 8 aa, homo sapiens
QQYGRSFT

SEQ ID NO: 72 - protein, 8 aa, homo sapiens
GGSFSGYY

SEQ ID NO: 73 - protein, 5 aa, homo sapiens
INQSG

SEQ ID NO: 74 - protein, 7 aa, homo sapiens
IQQSGST

SEQ ID NO: 75 - protein, 11 aa, homo sapiens
ASGNWDHFFDY

SEQ ID NO: 76 - protein, 6 aa, homo sapiens
QGISSW

SEQ ID NO: 77 - protein, 9 aa, homo sapiens
QQAKSFPWT

SEQ ID NO: 78 - protein, 8 aa, homo sapiens
GGSFSGYH

SEQ ID NO: 79 - protein, 7 aa, homo sapiens
ISHSGRT

SEQ ID NO: 80 - protein, 17 aa, homo sapiens
ASFITMIRGTIITHFDY

SEQ ID NO: 81 - protein, 6 aa, homo sapiens
QGISSW

SEQ ID NO: 82 - protein, 9 aa, homo sapiens
QQYHSYPYT

SEQ ID NO: 83 - protein, 8 aa, homo sapiens
GGTFSSYA

SEQ ID NO: 84 - protein, 8 aa, homo sapiens
IIPIFGIA

SEQ ID NO: 85 - protein, 17 aa, homo sapiens
ARRGDYYGSGSPDVFDI

SEQ ID NO: 86 - protein, 7 aa, homo sapiens
QSVSSSY

SEQ ID NO: 87 - protein, 8 aa, homo sapiens
QQYGSSYT

SEQ ID NO: 88 - protein, 8 aa, homo sapiens
GGTFSSYA

SEQ ID NO: 89 - protein, 8 aa, homo sapiens
IIPIFGIA

SEQ ID NO: 90 - protein, 17 aa, homo sapiens
ARRGNYYGSGSPDVFDI

SEQ ID NO: 91 - protein, 7 aa, homo sapiens
QSVSSSY

SEQ ID NO: 92 - protein, 8 aa, homo sapiens
QQYGSSYT

SEQ ID NO: 93 - protein, 8 aa, homo sapiens
GGTFSSYA

SEQ ID NO: 94 - protein, 8 aa, homo sapiens
IIPIFGIV

SEQ ID NO: 95 - protein, 17 aa, homo sapiens
ARRGNYYGSGSPDVFDI

SEQ ID NO: 96 - protein, 7 aa, homo sapiens
QSVSSSY

SEQ ID NO: 97 - protein, 8 aa, homo sapiens
QQYGSSYT

SEQ ID NO: 98 - protein, 8 aa, homo sapiens
GGTFSSYA

SEQ ID NO: 99 - protein, 8 aa, homo sapiens
IIPIFGIV

SEQ ID NO: 100 - protein, 17 aa, homo sapiens
ARRGNYYGSGSPDVFDI

SEQ ID NO: 101 - protein, 6 aa, homo sapiens
QSVSSY

SEQ ID NO: 102 - protein, 8 aa, homo sapiens
QQRSNWLT

SEQ ID NO: 103 - protein, 8 aa, homo sapiens
GGTFSSYA

SEQ ID NO: 104 - protein, 8 aa, homo sapiens
IIPIFGIA

SEQ ID NO: 105 - protein, 18 aa, homo sapiens
ARRGNYYGSGSPDVFDIS

SEQ ID NO: 106 - protein, 7 aa, homo sapiens
QSVSSSY

SEQ ID NO: 107 - protein, 8 aa, homo sapiens
QQYGSSYT

SEQ ID NO: 108 - protein, 8 aa, homo sapiens
GGSFSGYY

SEQ ID NO: 109 - protein, 7 aa, homo sapiens
ISHSGRT

SEQ ID NO: 110 - protein, 17 aa, homo sapiens
ARFITMIRGAIITHFDY

SEQ ID NO: 111 - protein, 17 aa, homo sapiens
ARFITLIRGAIITHFDY

SEQ ID NO: 112 - protein, 6 aa, homo sapiens
QGISSW

SEQ ID NO: 113 - protein, 9 aa, homo sapiens
QQYHSYPYT

SEQ ID NO: 114 - protein, 8 aa, homo sapiens
GFSFSTYA

SEQ ID NO: 115 - protein, 8 aa, homo sapiens
ISYDGDNK

SEQ ID NO: 116 - protein, 13 aa, homo sapiens
ARGRKLGIDAFDI

SEQ ID NO: 117 - protein, 6 aa, homo sapiens
QGISSA

SEQ ID NO: 118 - protein, 9 aa, homo sapiens
QQFNSYPFT

SEQ ID NO: 119 - protein, 8 aa, homo sapiens
  MISC_FEATURE (6)..(6): Wherein X is A or G
ISISGXST

SEQ ID NO: 120 - protein, 13 aa, homo sapiens
  Misc (13)..(13): Wherein X is I of F
RGYSGYVYDAFDX

SEQ ID NO: 121 - protein, 8 aa, homo sapiens
  MISC_FEATURE (8)..(8): Wherein X is I or F
GGSFSGYX

SEQ ID NO: 122 - protein, 17 aa, homo sapiens
  MISC_FEATURE (2)..(2): Wherein X is S or R
  MISC_FEATURE (10)..(10): Wherein X is T or A
AXFITMIRGXIITHFDY

SEQ ID NO: 123 - protein, 8 aa, homo sapiens
  MISC_FEATURE (6)..(6): Wherein X is S or N
GFTFSXYA

SEQ ID NO: 124 - protein, 8 aa, homo sapiens
ISVSGGST

SEQ ID NO: 125 - protein, 18 aa, homo sapiens
AKEGYIWFGESLSYAFDI

SEQ ID NO: 126 - protein, 8 aa, homo sapiens
  MISC_FEATURE (8)..(8): Wherein X is A or V
IIPIFGIX

SEQ ID NO: 127 - protein, 17 aa, homo sapiens
  MISC_FEATURE (5)..(5): Wherein X is D or N
ARRGXYYGSGSPDVFDI

SEQ ID NO: 128 - protein, 7 aa, homo sapiens
  MISC_FEATURE (4)..(4): Wherein X is S or deleted
QSVXSSY

SEQ ID NO: 129 - protein, 8 aa, homo sapiens
  MISC_FEATURE (3)..(3): Wherein X is R or Y
  MISC_FEATURE (4)..(4): Wherein X is Sor G
  MISC_FEATURE (4)..(4): Wherein X is Sor G
  MISC_FEATURE (5)..(5): Wherein X is N or S
  MISC_FEATURE (6)..(6): Wherein X is W or S
  MISC_FEATURE (6)..(6): Wherein X is W or S
  MISC_FEATURE (7)..(7): Wherein X is L or Y
QQXXXXXT

SEQ ID NO: 130 - protein, 894 aa, homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 131 - protein, 894 aa, Artificial sequence
  Homo sapiens AXL - Mus musculus Ig1 domain
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDKAVPANTPFNLSCQAQGPPEPVTLLWLQDAVPLAPVTGHSSQHSLQTPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 132 - protein, 894 aa, Artificial sequence; identical to SEQ ID NO: 131
  Homo sapiens AXL - Mus musculus Ig2 domain

SEQ ID NO: 133 - protein, 560 aa, Artificial sequence
  Homo sapiens AXL - Mus musculus FN1 domain
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQRPHHLHVVSRQPTELEVAWTPGLSGIYPLTHCNLQAVLSDDGVGIWLGKSDPPEDPLTLQVSVPPHQLRLEKLLPHTPYHIRISCSSSQGPSPWTHWLPVETTEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDS

SEQ ID NO: 134 - protein, 894 aa, Artificial sequence
  Homo sapiens AXL - Mus musculus FN2 domain
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENVSAMRNGSQVLVRWQEPRVPLQGTLLGYRLAYRGQDTPEVLMDIGLTREVTLELRGDRPVANLTVSVTAYTSAGDGPWSLPVPLEPWRPGQGQPLHHLVSEPPPRAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 135 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 136 - protein, 290 aa, Homa sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 137 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 138 - protein, 677 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 139 - protein, 145 aa, Artificial sequence
  Synthetic heavy chain variable region 1H12VH
MGFKMESQFQVFVFVFLWLSGVDGEVQLVESGGDLVKPGGSLKLSCAASGFTFSSYGMSWVRQTPDKRLEWVATISSGGSYTYYPDSVKGRFTSRDNAKNTLYLQMSSLKSEDTAMYYCARHPIYYTYDDTMDYWGQGTSVTVSS

SEQ ID NO: 140 - protein, 131 aa, Artificial sequence
  Synthetic light chain variable region 1H12VK
MGFKMESQFQVFVFVFLWLSGVDGENVLTQSPAIMAASPGEKVTTCSASSSVSSGNFHWYQQKPGTSPKLWIYRTSNLASGVPARFSGSGSGTSYSLTISSMEAEDAATYYCQQWSGYPWTFGGGTKLEIK

SEQ ID NO: 141 - protein, 144 aa, Artificial sequence
  Synthetic heavy chain variable region 1H12RHA
MGFKMESQFQVFVFVFLWLSGVDGQVQLVESGGGWQPGRSLRLSCAASGFTFSSYGMSVRQAPGKGLEWVATISSGGSYTYYPDSVKGRFTISRDNSKNTLYLQMNSLRAEDTAVYYCARHPIYYTYDDTMDYWGQGTTVTVSS

SEQ ID NO: 142 - protein, 132 aa, Artificial sequence
  Synthetic light chain variable region 1H12RKA
MGFKMESQFQVFVFVFLWLSGVDGEIVLTQSPATLSLSPGERATLSCSASSSVSSGNFHWYQQKPGLAPRLLIYRTSNLASGIPDRFSGSGSGTDFTLTISRLEPEDFAVYYCQQWSGYPWTFGPGTKVDIK

SEQ ID NO: 143 - protein, 131 aa, Artificial sequence
  Synthetic light chain variable region 1H12RKB
MGFKMESQFQVFVFVFLWLSGVDGEIVLTQSPGTLSLSPGERATLSCSASSSVSSGNFHWYQQKPGLAPRLLIYRTSNLASGIPARFSGSGSGTDFTLTSSLEPEDFAVYYCQQWSGYPWTFGGGTKLEIK

SEQ ID NO: 144 - protein, 146 aa, Artificial sequence
  Synthetic heavy chain variable region 1H12RHB
MGFKMESQFQVFVFVFLWLSGVDGEVQLVESGGGLVQPGGSLRLSCAASGFTFSSYGMSWVRQAPGKGLEWVATISSGGSYTYYPDSVKGRFTISRDNAKNSLYLQMNSLRAEDTAVYYCARHPIYYTYDDTMDYWGQGTLVTVSS

SEQ ID NO: 145 - DNA, 360 bp, Artificial sequence
  Synthetic sequence
gaggtccagctggtacagtctggggctgaggtgaagaagcctggggctacagtgaaaatctcctgcaaggtttctggttactcattcactggcgctaccatgaactggatccgccagcccccagggaaggggctggagtggattggtcttattaaaccttccaatggtggtactagttacaaccagaagttcaagggcagagtcaccatctcagccgacaagtccatcagcaccgcctacctgcagtggagcagcctgaaggcctcggacaccgccatgtattactgtgcacatggtcactacgagagttacgaggctatggactactggggccagggaacgctggtcaccgtcagctca

SEQ ID NO: 146 - DNA, 323 bp, Artificial sequence
  Synthetic sequence
gacatccagatgacccagtctccatcctccctgtctgcatctgtaggagacagagtcaccatcacttgcaaggccagtcaggatgtggtttctgctgtagcctggtaccagcagaaacctggccaggctcccaggctcctcatctattggcaggatacccggcacactggagtcccatcaaggttcagcggcagtggatctgggacagaattcactctcaccatcagcagcctgcagcctgatgattttgcaacttattactgtcaggaacattttagcactccgctcacgttcggccaagggaccaaggtggaaatcaaacc

SEQ ID NO: 147 - DNA, 322 bp, Artificial sequence
  Synthetic sequence
gacatccagatgacccagtctccatcctccctgtctgcatctgtaggagacagagtcaccatcacttgcaaggccagtcaggatgtgagttctgctgtagcctggtaccagcagaaacctggccaggctcccaggctcctcatctattggcaggatacccggcacactggagtcccatcaaggttcagcggcagtggatctgggacagaattcactctcaccatcagcagcctgcagcctgatgattttgcaacttattactgtcaggaacattttagccctccgctcacgttcggccaagggaccaaggtggaaatcaaac

SEQ ID NO: 148 - DNA, 323 bp, Artificial seequence
  Synthetic sequence
gacatccagatgacccagtctccatcctccctgtctgcatctgtaggagacagagtcaccatcacttgcaaggccagtcaggatgtggtttctgctgtagcctggtaccagcagaaacctggccaggctcccaggctcctcatctattggcaggatacccggcacactggagtcccatcaaggttcagcggcagtggatctgggacagaattcactctcaccatcagcagcctgcagcctgatgattttgcaacttattactgtcaggaacattttagccctccgctcacgttcggccaagggaccaaggtggaaatcaaacc

SEQ ID NO: 149 - DNA, 323 bp, Artificial sequence
  Synthetic sequence
gacatccagatgacccagtctccatcctccctgtctgcatctgtaggagacagagtcaccatcacttgcaaggccagtcaggatgtggtttctgctgtagcctggtaccagcagaaacctggccaggctcccaggctcctcatctattggcaggatacccggcacactggagtcccatcaaggttcagcggcagtggatctgggacagaattcactctcaccatcagcagcctgcagcctgatgattttgcaacttattactgtcaggaacattttagccctccgctcaggttcggccaagggaccaaggtggaaatcaaacc

SEQ ID NO: 150 - DNA, 360 bp, Artificial sequence
  Synthetic sequence
gaggtccagctggtacagtctggggctgaggtgaagaagcctggggctacagtgaaaatctcctgcaaggtttctggttactcattctggggcgctaccatgaactggatccgccagcccccagggaaggggctggagtggattggtcttattaaaccttccaatggtggtactagttacaaccagaagttcaagggcagagtcaccatctcagccgacaagtccatcagcaccgcctacctgcagtggagcagcctgaaggcctcggacaccgccatgtattactgtgcacatggtcactacgagagttacgaggctatggactactggggccagggaacgctggtcaccgtcagctca

SEQ ID NO: 151 - DNA, 360 bp, Artificial sequence
  synthetic sequence
gaggtccagctggtacagtctggggctgaggtgaagaagcctggggctacagtgaaaatctcctgcaaggtttctggttactcattcactggccacaccatgaactggatccgccagcccccagggaaggggctggagtggattggtcttattaaaccttccaatggtggtactagttacaaccagaagttcaagggcagagtcaccatctcagccgacaagtccatcagcaccgcctacctgcagtggagcagcctgaaggcctcggacaccgccatgtattactgtgcacatggtcactacgagagttacgaggctatggactactggggccagggaacgctggtcaccgtcagctca

SEQ ID NO: 152 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 107 aa, Homo sapiens [sequence omitted]

SEQ ID NO: 154 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS